(19) United States
(12) Patent Application Publication
(10) Pub. No.: US 2024/0041861 A1
(43) Pub. Date: Feb. 8, 2024

(54) COMPOSITIONS AND METHODS FOR TREATMENT AND PROPHYLAXIS OF VIRAL INFECTIONS

(71) Applicant: NED Biosystems, Inc., Cambridge, MA (US)

(72) Inventors: Rebecca Lambert Bent, Washington, CT (US); Peter K. Cabeceiras, Boston, MA (US)

(21) Appl. No.: 17/920,385

(22) PCT Filed: Apr. 21, 2021
(86) PCT No.: PCT/US2021/028385

§ 371 Date: Oct. 20, 2022

Related U.S. Application Data
(60) Provisional application No. 63/013,532, filed on Apr. 21, 2020.
(60) Provisional application No. 63/031,532, filed on May 28, 2020.

(51) Int. Cl. A61K 31/485; A61K 31/385; A61K 31/12; A61K 31/352; A61K 31/407; A61K 31/155; A61K 45/06; A61P 31/14

(52) U.S. Cl. CPC A61K 31/485; A61K 31/385; A61K 31/12; A61K 31/352; A61K 31/407; A61K 31/155; A61K 45/06; A61P 31/14

(57) ABSTRACT

The present disclosure is directed to compositions and methods for the treatment or prevention of viral infections and diseases or conditions associated with viral infections. Among other things, the present disclosure encompasses the insight that redundant targeting of multiple pathways essential for viral infection and replication provides effective treatment and prevention of viral infection.

### RELATED APPLICATIONS

This application claims priority to U.S. Provisional Application No. 63/013,532, filed Apr. 21, 2020, and U.S. Provisional Application No. 63/031,532, filed May 28, 2020, the entirety of both applications is hereby incorporated herein by reference.

### BACKGROUND

Newly emerging viruses such as Ebola virus, SARS coronavirus, MERS coronavirus, and avian influenza virus pose serious threats to the global public health and economy. Since the 1970s, viruses of unknown origin have been continuously discovered all over the world. The first outbreak of the Ebola virus occurred in 1976 in Zaire and Sudan. AIDS was first described as acquired immunodeficiency syndrome in 1981, and its viral cause was eventually identified as HIV several years later. SARS, a respiratory disease caused by novel coronavirus SARS-CoV, was first reported in Hong Kong in 2003 and led to at least 775 deaths in multiple countries. Another novel human coronavirus, Middle East respiratory syndrome-coronavirus (MERS-CoV), was discovered in 2012 in Saudi Arabia, and other cases were reported in 22 other countries in June 2015. More recently, the novel human coronavirus SARS-CoV-2, was identified in Wuhan, China in December 2019 and led to a global pandemic. In April 2020, more than 1.51 million cases were reported in more than 200 countries and territories, resulting in more than 88,300 deaths. There is an urgent need for therapeutics and prophylactics to combat highly infectious and pathogenic viruses.

### SUMMARY

The present disclosure encompasses the recognition that many processes or pathways become altered in an individual suffering from a viral infection as compared to the same processes or pathways as they function in normal individuals not suffering from a viral infection. The present disclosure further encompasses the recognition that pathways that become altered as a result of a viral infection, and in particular an RNA virus infection, overlap with certain pathways that advance cancers. In particular, the present disclosure identifies four pathways affected by viral infection (e.g., infection with an RNA virus) including immune response, apoptosis, metabolism, and/or angiogenesis.

The present disclosure is directed to compositions and methods for the treatment or prevention of viral infections and diseases or conditions associated with viral infections. Among other things, the present disclosure encompasses the insight that redundant targeting of multiple pathways essential for viral infection and replication provides effective treatment and prevention of virus associated diseases, disorders, and conditions.

In some embodiments, the present disclosure provides methods of treating or preventing a viral infection, e.g., an RNA virus infection, in a subject, the methods comprising a step of administering to the subject a combination of agents, wherein the combination of agents comprises three or more of alpha lipoic acid, curcumin, cyclophosphamide, genistein, melatonin, metformin, and naltrexone. In some embodiments, the combination of agents comprises four, five, or six of alpha lipoic acid, curcumin, cyclophosphamide, genistein, melatonin, metformin, and naltrexone. Those skilled in the art, reading the present specification, will appreciate that, in some embodiments, it may be desirable to omit cyclophosphamide, or replace cyclophosphamide with a comparable agent, as when dosed at high levels, cyclophosphamide can have associated toxicities, e.g., blood clotting, stroke, among others. Thus, in some embodiments, provided technologies utilize a combination of agents that comprises three or more of alpha lipoic acid, curcumin, genistein, melatonin, metformin, and naltrexone.

In some embodiments, the present disclosure provides methods of treating or preventing a viral infection, e.g., an RNA virus infection, in a subject, the methods comprising a step of administering to the subject a combination of agents, wherein the combination of agents comprises three or more of alpha lipoic acid, curcumin, genistein, melatonin, metformin, and naltrexone.

In some embodiments, the present disclosure provides methods of treating or preventing a disease or condition associated with a viral infection in a subject, the methods comprising a step of administering to the subject a combination of agents, wherein the combination of agents comprises three or more of alpha lipoic acid, curcumin, genistein, melatonin, metformin, and naltrexone.

In some preferred embodiments, a disease or condition associated with a viral infection is coronavirus disease 2019 (COVID-19).

In some embodiments, a viral infection is caused by a virus of a viral family selected from Reovirus, Picornavirus, Coronavirus, Flavivirus, Togavirus, Filovirus, Orthomyxovirus, Rhabdovirus, and Retrovirus.

In some particular embodiments, a virus is a negative-sense single stranded RNA virus.

In some embodiments, a virus is a coronavirus.

In some particularly preferred embodiments, a virus is SARS-CoV-2.

In some embodiments, a subject is infected with virus prior to receiving treatment.

In some embodiments, a subject receives treatment prior to exposure to virus.

In some embodiments, a agents are administered to a subject according to a personalized weekly or monthly regimen.

In some embodiments, a combination of agents comprises four or more of alpha lipoic acid, curcumin, genistein, melatonin, metformin, and naltrexone.

In some embodiments, a combination of agents comprises five or more of alpha lipoic acid, curcumin, genistein, melatonin, metformin, and naltrexone.

In some embodiments, a combination of agents comprises six or more of alpha lipoic acid, curcumin, cyclophosphamide, genistein, melatonin, metformin, and naltrexone.

In some embodiments, a combination of agents comprises alpha lipoic acid, curcumin, genistein, melatonin, metformin, and naltrexone.

In some embodiments, combination further comprises one or more additional anti-viral therapies.

In some embodiments, a combination further comprises one or more of Abacavir sulfate, Acyclovir, Bevacizumab, Bromelain, Bromhexine hydrochloride, Chloroquine phosphate, Danoprevir, Delaviridine mesylate, Didanosine, Dipyridamole, Ebastine, Entecavir, Favipiravir, Ganciclovir sodium, Hydroxychloroquine sulfate, Interferon alpha, Intravenous immunoglobulin, Lamivudine, Lopinavir, Ritonavir, Methylprednisolone, Nelfinavir mesylate, Nivolumab, Recombinant human ACE2, Remdesivir, Remestemcel-L, Saquinavir mesylate, Sildenafile citrate, Stavudine, Tenofovir disoproxil fumarate, Thymosin, Thalidomide, Umifenovir, Zanamivir, Zidovudine, Niclosamide, Famotidine, and combination thereof.

In some embodiments, a combination comprises alpha lipoic acid at a dose of about 600-2400 mg/day. In some embodiments, a combination comprises alpha lipoic acid at a dose of at least 500 mg/day, at least 600 mg/day, at least 700 mg/day, at least 800 mg/day, at least 900 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, at least 1800 mg/day, at least 1900 mg/day, at least 2000 mg/day, at least 2100 mg/day, at least 2200 mg/day, or at least 2300 mg/day. In some embodiments, a combination comprises alpha lipoic acid at a dose of at most 2700 mg/day, at most 2600 mg/day, at most 2500 mg/day, at most 2400 mg/day, at most 2300 mg/day, at most 2200 mg/day, at most 2100 mg/day, at most 2000 mg/day, at most 1900 mg/day, at most 1800 mg/day, at most 1700 mg/day, at most 1600 mg/day, at most 1500 mg/day, at most 1400 mg/day, at most 1300 mg/day, at most 1200 mg/day, at most 1100 mg/day, at most 1000 mg/day, at most 900 mg/day, at most 800 mg/day, or at most 700 mg/day.

In some embodiments, a combination comprises alpha lipoic acid at a dose of about 1200 mg/day.

In some embodiments, a combination comprises curcumin at a dose of about 200-3000 mg/day. In some embodiments, a combination comprises curcumin at a dose of at least 100 mg/day, at least 200 mg/day, at least 300 mg/day, at least 400 mg/day, at least 500 mg/day, at least 600 mg/day, at least 700 mg/day, at least 800 mg/day, at least 900 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, at least 1800 mg/day, at least 1900 mg/day, at least 2000 mg/day, at least 2100 mg/day, at least 2200 mg/day, at least 2300 mg/day, at least 2400 mg/day, or at least 2500 mg/day. In some embodiments, a combination comprises curcumin at a dose of at most 3000 mg/day, at most 2900 mg/day, at most 2800 mg/day, at most 2700 mg/day, at most 2600 mg/day, at most 2500 mg/day, at most 2400 mg/day, at most 2300 mg/day, at most 2200 mg/day, at most 2100 mg/day, at most 2000 mg/day, at most 1500 mg/day, at most 1400 mg/day, at most 1300 mg/day, at most 1200 mg/day, at most 1100 mg/day, at most 1000 mg/day, at most 900 mg/day at most 800 mg/day, at most 700 mg/day, at most 600 mg/day, at most 500 mg/day, at most 400 mg/day, or at most 300 mg/day.

In some embodiments, a combination comprises curcumin at a dose of about 1500 mg/day.

In some embodiments, a combination comprises genistein at a dose of about 60-1000 mg/day. In some embodiments, a combination comprises genistein at a dose of at least 50 mg/day, at least 60 mg/day, at least 70 mg/day, at least 80 mg/day, at least 90 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, or at least 950 mg/day. In some embodiments, a combination comprises genistein at a dose of at most 1200 mg/day, at most 1100 mg/day, at most 1000 mg/day, at most 950 mg/day, at most 900 mg/day, at most 850 mg/day, at most 800 mg/day, at most 750 mg/day, at most 700 mg/day, at most 650 mg/day, at most 600 mg/day, at most 550 mg/day, at most 500 mg/day, at most 450 mg/day, at most 400 mg/day, at most 350 mg/day, at most 300 mg/day, at most 250 mg/day, at most 200 mg/day, at most 150 mg/day, or at most 100 mg/day.

In some embodiments, a combination comprises genistein at a dose of about 500 mg/day.

In some embodiments, a combination comprises metformin at a dose of about 250-2000 mg/day. In some embodiments, a combination comprises metformin at a dose of at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, or at least 1800 mg/day. In some embodiments, a combination comprises metformin at a dose of at most 2500 mg/day, at most 2000 mg/day, at most 1750 mg/day, at most 1500 mg/day, at most 1250 mg/day, at most 1000 mg/day, at most 750 mg/day, at most 500 mg/day, at most 450 mg/day, at most 400 mg/day, at most 350 mg/day, or at most 300 mg/day.

In some embodiments, a combination comprises metformin at a dose of about 1000 mg/day.

In some embodiments, a combination comprises melatonin at a dose of about 1-40 mg/day. In some embodiments, a combination comprises melatonin at a dose of at least 0.5 mg/day, at least 1 mg/day, at least 5 mg/day, at least 10 mg/day, at least 15 mg/day, at least 20 mg/day, at least 25 mg/day, at least 30 mg/day, or at least 35 mg/day. In some embodiments, a combination comprises melatonin at a dose of at most 50 mg/day, at most 45 mg/day, at most 40 mg/day, at most 35 mg/day, at most 30 mg/day, at most 25 mg/day, at most 20 mg/day, at most 15 mg/day, at most 10 mg/day, or at most 5 mg/day.

In some embodiments, a combination comprises melatonin at a dose of about 10 mg/day.

In some embodiments, a combination comprises naltrexone at a dose of about 1.75-7 mg/day. In some embodiments, a combination comprises naltrexone at a dose of at least 0.5 mg/day, at least 1 mg/day, at least 1.5 mg/day, at least 2 mg/day, at least 2.5 mg/day, at least 3 mg/day, at least 3.5 mg/day, at least 4 mg/day, at least 4.5 mg/day, at least 5 mg/day, at least 5.5 mg/day, at least 6 mg/day, or at least 6.5 mg/day. In some embodiments, a combination comprises naltrexone at a dose of at most 10 mg/day, at most 9 mg/day, at most 8 mg/day, at most 7 mg/day, at most 6 mg/day, at most 5 mg/day, at most 4.5 mg/day, at most 4 mg/day, at most 3.5 mg/day, at most 3 mg/day, at most 2.5 mg/day, or at most 2 mg/day.

In some embodiments, a combination comprises naltrexone at a dose of about 3.5 mg/day.

In some embodiments, a combination comprises cyclophosphamide. In some embodiments, a combination comprises cyclophosphamide at a dose of about 25-50 mg/day. In some embodiments, a combination comprises cyclophosphamide at a dose of at least 15 mg/day, at least 20 mg/day, at least 25 mg/day, at least 30 mg/day, at least 35 mg/day, at least 40 mg/day, or at least 45 mg/day. In some embodiments, a combination comprises cyclophosphamide at a dose of at most 75 mg/day, at most 70 mg/day, at most 65 mg/day, at most 60 mg/day, at most 55 mg/day, at most 50 mg/day, at most 45 mg/day, at most 40 mg/day, at most 35 mg/day, or at most 30 mg/day. In some embodiments, combination comprises cyclophosphamide at a dose of about 50 mg/day.

In some embodiments, one or more of the agents is formulated for oral administration.

In some embodiments, one or more agents are formulated for buccal administration, sub-labial administration, sub-lingual administration, or a combination thereof.

In some embodiments, one or more agents are formulated for parenteral administration.

In some embodiments, parenteral administration includes subcutaneous injection, intramuscular injection, intravenous injection, or combinations thereof.

In some embodiments, two or more agents are formulated together as an admixture.

In some embodiments, agents are formulated together as an admixture.

In some embodiments, agents are formulated as separate co-agents.

In some embodiments, a combination includes a pharmaceutically acceptable carrier, diluent, or excipient.

In some embodiments, agents are each in the form of tablet, powder, or liquid.

In some embodiments, a treatment is administered to a subject until a viral infection, or disease or condition associated with a viral infection is addressed.

In some embodiments, a viral infection is addressed when oxygen saturation rates are improved.

In some embodiments, a viral infection is addressed when symptomatic shortness of breath is improved.

In some embodiments, a viral infection is addressed when inflammatory markers indicate clinically relevant improvement.

In some embodiments, a viral infection is addressed when white blood cell count is improved.

Among other things, the present disclosure provides a combination comprising: (i) three or more agents comprising alpha lipoic acid, curcumin, genistein, melatonin, metformin, and naltrexone; and (ii) one or more additional anti-viral therapies.

In some embodiments, one or more additional anti-viral therapies comprise Abacavir sulfate, Acyclovir, Bevacizumab, Bromelain, Bromhexine hydrochloride, Chloroquine phosphate, Danoprevir, Delaviridine mesylate, Didanosine, Dipyridamole, Ebastine, Entecavir, Favipiravir, Ganciclovir sodium, Hydroxychloroquine sulfate, Interferon alpha, Intravenous immunoglobulin, Lamivudine, Lopinavir, Ritonavir, Methylprednisolone, Nelfinavir mesylate, Nivolumab, Recombinant human ACE2, Remdesivir, Remestemcel-L, Saquinavir mesylate, Sildenafile citrate, Stavudine, Tenofovir disoproxil fumarate, Thymosin, Thalidomide, Umifenovir, Zanamivir, Zidovudine, Niclosamide, Famotidine, and combination thereof.

In some embodiments, a combination comprises alpha lipoic acid at a dose of about 600-2400 mg/day. In some embodiments, a combination comprises alpha lipoic acid at a dose of at least 500 mg/day, at least 600 mg/day, at least 700 mg/day, at least 800 mg/day, at least 900 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, at least 1800 mg/day, at least 1900 mg/day, at least 2000 mg/day, at least 2100 mg/day, at least 2200 mg/day, or at least 2300 mg/day. In some embodiments, a combination comprises alpha lipoic acid at a dose of at most 2700 mg/day, at most 2600 mg/day, at most 2500 mg/day, at most 2400 mg/day, at most 2300 mg/day, at most 2200 mg/day, at most 2100 mg/day, at most 2000 mg/day, at most 1900 mg/day, at most 1800 mg/day, at most 1700 mg/day, at most 1600 mg/day, at most 1500 mg/day, at most 1400 mg/day, at most 1300 mg/day, at most 1200 mg/day, at most 1100 mg/day, at most 1000 mg/day, at most 900 mg/day, at most 800 mg/day, or at most 700 mg/day.

In some embodiments, a combination comprises alpha lipoic acid at a dose of about 1200 mg/day.

In some embodiments, a combination comprises curcumin at a dose of about 200-3000 mg/day. In some embodiments, a combination comprises curcumin at a dose of at least 100 mg/day, at least 200 mg/day, at least 300 mg/day, at least 400 mg/day, at least 500 mg/day, at least 600 mg/day, at least 700 mg/day, at least 800 mg/day, at least 900 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, at least 1800 mg/day, at least 1900 mg/day, at least 2000 mg/day, at least 2100 mg/day, at least 2200 mg/day, at least 2300 mg/day, at least 2400 mg/day, or at least 2500 mg/day. In some embodiments, a combination comprises curcumin at a dose of at most 3000 mg/day, at most 2900 mg/day, at most 2800 mg/day, at most 2700 mg/day, at most 2600 mg/day, at most 2500 mg/day, at most 2400 mg/day, at most 2300 mg/day, at most 2200 mg/day, at most 2100 mg/day, at most 2000 mg/day, at most 1500 mg/day, at most 1400 mg/day, at most 1300 mg/day, at most 1200 mg/day, at most 1100 mg/day, at most 1000 mg/day, at most 900 mg/day at most 800 mg/day, at most 700 mg/day, at most 600 mg/day, at most 500 mg/day, at most 400 mg/day, or at most 300 mg/day.

In some embodiments, a combination comprises curcumin at a dose of about 1500 mg/day.

In some embodiments, a combination comprises genistein at a dose of about 60-1000 mg/day. In some embodiments, a combination comprises genistein at a dose of at least 50 mg/day, at least 60 mg/day, at least 70 mg/day, at least 80 mg/day, at least 90 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, or at least 950 mg/day. In some embodiments, a combination comprises genistein at a dose of at most 1200 mg/day, at most 1100 mg/day, at most 1000 mg/day, at most 950 mg/day, at most 900 mg/day, at most 850 mg/day, at most 800 mg/day, at most 750 mg/day, at most 700 mg/day, at most 650 mg/day, at most 600 mg/day, at most 550 mg/day, at most 500 mg/day, at most 450 mg/day, at most 400 mg/day, at most 350 mg/day, at most 300 mg/day, at most 250 mg/day, at most 200 mg/day, at most 150 mg/day, or at most 100 mg/day.

In some embodiments, a combination comprises genistein at a dose of about 500 mg/day.

In some embodiments, a combination comprises metformin at a dose of about 250-2000 mg/day. In some embodiments, a combination comprises metformin at a dose of at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, or at least 1800 mg/day. In some embodiments, a combination comprises metformin at a dose of at most 2500 mg/day, at most 2000 mg/day, at most 1750 mg/day, at most 1500 mg/day, at most 1250 mg/day, at most 1000 mg/day, at most 750 mg/day, at most 500 mg/day, at most 450 mg/day, at most 400 mg/day, at most 350 mg/day, or at most 300 mg/day.

In some embodiments, a combination comprises metformin at a dose of about 1000 mg/day.

In some embodiments, a combination comprises melatonin at a dose of about 1-40 mg/day. In some embodiments, a combination comprises melatonin at a dose of at least 0.5 mg/day, at least 1 mg/day, at least 5 mg/day, at least 10 mg/day, at least 15 mg/day, at least 20 mg/day, at least 25 mg/day, at least 30 mg/day, or at least 35 mg/day. In some embodiments, a combination comprises melatonin at a dose of at most 50 mg/day, at most 45 mg/day, at most 40 mg/day, at most 35 mg/day, at most 30 mg/day, at most 25 mg/day, at most 20 mg/day, at most 15 mg/day, at most 10 mg/day, or at most 5 mg/day.

In some embodiments, a combination comprises melatonin at a dose of about 10 mg/day.

In some embodiments, a combination comprises naltrexone at a dose of about 1.75-7 mg/day. In some embodiments, a combination comprises naltrexone at a dose of at least 0.5 mg/day, at least 1 mg/day, at least 1.5 mg/day, at least 2 mg/day, at least 2.5 mg/day, at least 3 mg/day, at least 3.5 mg/day, at least 4 mg/day, at least 4.5 mg/day, at least 5 mg/day, at least 5.5 mg/day, at least 6 mg/day, or at least 6.5 mg/day. In some embodiments, a combination comprises naltrexone at a dose of at most 10 mg/day, at most 9 mg/day, at most 8 mg/day, at most 7 mg/day, at most 6 mg/day, at most 5 mg/day, at most 4.5 mg/day, at most 4 mg/day, at most 3.5 mg/day, at most 3 mg/day, at most 2.5 mg/day, or at most 2 mg/day.

In some embodiments, a combination comprises naltrexone at a dose of about 3.5 mg/day.

In some embodiments, a combination comprises cyclophosphamide. In some embodiments, a combination comprises cyclophosphamide at a dose of about 25-50 mg/day. In some embodiments, a combination comprises cyclophosphamide at a dose of at least 15 mg/day, at least 20 mg/day, at least 25 mg/day, at least 30 mg/day, at least 35 mg/day, at least 40 mg/day, or at least 45 mg/day. In some embodiments, a combination comprises cyclophosphamide at a dose of at most 75 mg/day, at most 70 mg/day, at most 65 mg/day, at most 60 mg/day, at most 55 mg/day, at most 50 mg/day, at most 45 mg/day, at most 40 mg/day, at most 35 mg/day, or at most 30 mg/day. In some embodiments, combination comprises cyclophosphamide at a dose of about 50 mg/day.

In some embodiments, one or more of the agents is formulated for oral administration.

In some embodiments, one or more agents are formulated for buccal administration, sub-labial administration, sub-lingual administration, or a combination thereof.

In some embodiments, one or more agents are formulated for parenteral administration.

In some embodiments, parenteral administration includes subcutaneous injection, intramuscular injection, intravenous injection, or combinations thereof.

In some embodiments, two or more agents are formulated together as an admixture.

In some embodiments, agents are formulated together as an admixture.

In some embodiments, agents are formulated as separate co-agents.

In some embodiments, a combination includes a pharmaceutically acceptable carrier, diluent, or excipient.

In some embodiments, agents are each in the form of tablet, powder, or liquid.

In some embodiments, the present disclosure provides a blisterpak comprising a dosage of a combination, wherein the combination comprises: (i) three or more of alpha lipoic acid, curcumin, genistein, melatonin, metformin, and naltrexone; and (ii) one or more additional anti-viral therapies.

In some embodiments, one or more additional anti-viral therapies comprise Abacavir sulfate, Acyclovir, Bevacizumab, Bromelain, Bromhexine hydrochloride, Chloroquine phosphate, Danoprevir, Delaviridine mesylate, Didanosine, Dipyridamole, Ebastine, Entecavir, Favipiravir, Ganciclovir sodium, Hydroxychloroquine sulfate, Interferon alpha, Intravenous immunoglobulin, Lamivudine, Lopinavir, Ritonavir, Methylprednisolone, Nelfinavir mesylate, Nivolumab, Recombinant human ACE2, Remdesivir, Remestemcel-L, Saquinavir mesylate, Sildenafile citrate, Stavudine, Tenofovir disoproxil fumarate, Thymosin, Thalidomide, Umifenovir, Zanamivir, Zidovudine, Niclosamide, Famotidine, and combination thereof.

In some embodiments, the present disclosure provides for use of a combination for the treatment of a viral infection (e.g., an RNA virus infection) in a subject, wherein the combination comprises three or more of alpha lipoic acid, curcumin, genistein, melatonin, metformin, and naltrexone.

In some embodiments, the present disclosure provides for use of a combination for the treatment of a disease or condition associated with a viral infection in a subject, wherein the combination comprises three or more of alpha lipoic acid, curcumin, genistein, melatonin, metformin, and naltrexone.

In some embodiments, a disease or condition associated with a viral infection is coronavirus disease 2019 (COVID-19).

In some embodiments, a viral infection is caused by a virus of a viral family selected from Reovirus, Picornavirus, Coronavirus, Flavivirus, Togavirus, Filovirus, Orthomyxovirus, Rhabdovirus, and Retrovirus.

In some embodiments, a virus is a negative-sense single stranded RNA virus.

In some embodiments, a virus is a coronavirus.

In some embodiments, a virus is SARS-CoV-2.

In some embodiments, a subject is infected with virus prior to receiving treatment.

In some embodiments, a subject receives treatment prior to exposure to virus.

In some embodiments, the present disclosure provides for methods of treating or preventing a virus infection in a subject, the methods comprising a step of administering to the subject a combination of agents, wherein the combination of agents comprises three or more of alpha lipoic acid, curcumin, genistein, melatonin, metformin, and naltrexone.

In some embodiments, viral infection is caused by a virus of a viral family selected from Herpesvirus, Papovavirus, Adenovirus, Parvovirus, Poxvirus, Anellovirus, and Pleolipovirus.

Among other things, the present disclosure provides a combination comprising three or more of alpha lipoic acid, curcumin, genistein, melatonin, metformin, or naltrexone.

The present disclosure further provides a combination comprising alpha lipoic acid, curcumin, genistein, melatonin, metformin, and naltrexone.

### BRIEF DESCRIPTION OF THE DRAWING

FIG. 1 shows pleiotropic molecular targets of an embodiment according to the present disclosure.

FIG. 2 shows an exemplary cartoon depicting certain mechanisms of action (MoAs) for certain agents according to an embodiment of the present disclosure.

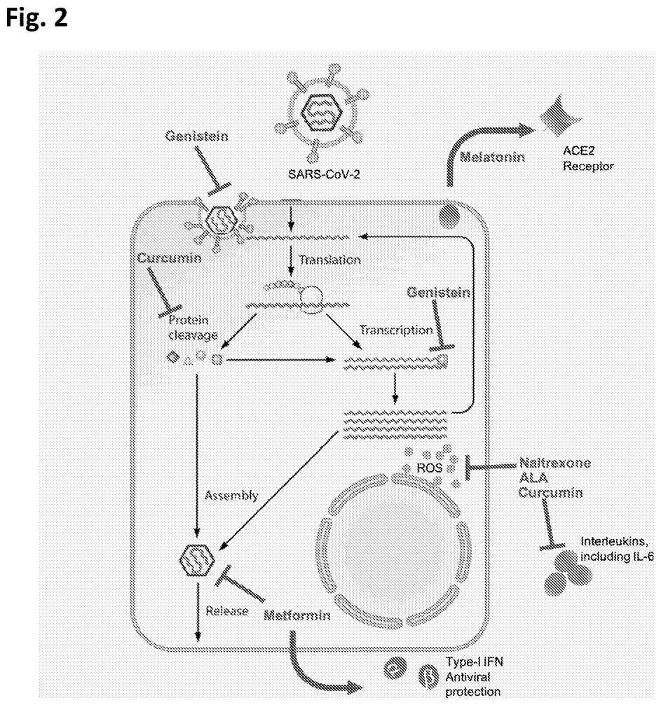

FIG. 3 shows an embodiment according to the present disclosure compared to leading antiviral compounds.

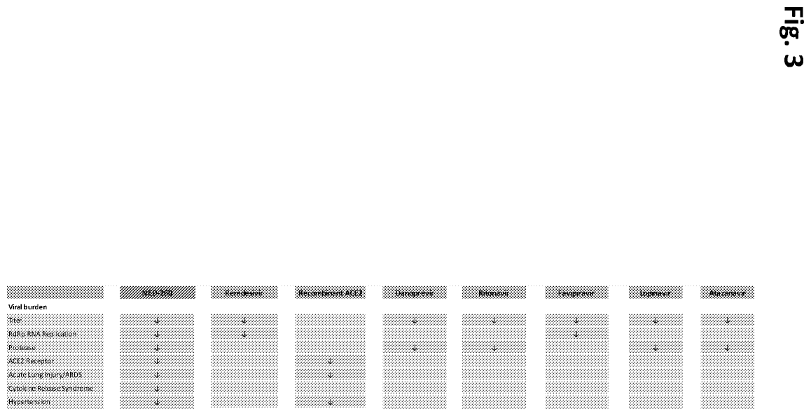

FIG. 4 shows an embodiment according to the present disclosure compared to leading repurposed compounds.

FIG. 5 shows an embodiment according to the present disclosure compared to leading repurposed compounds.

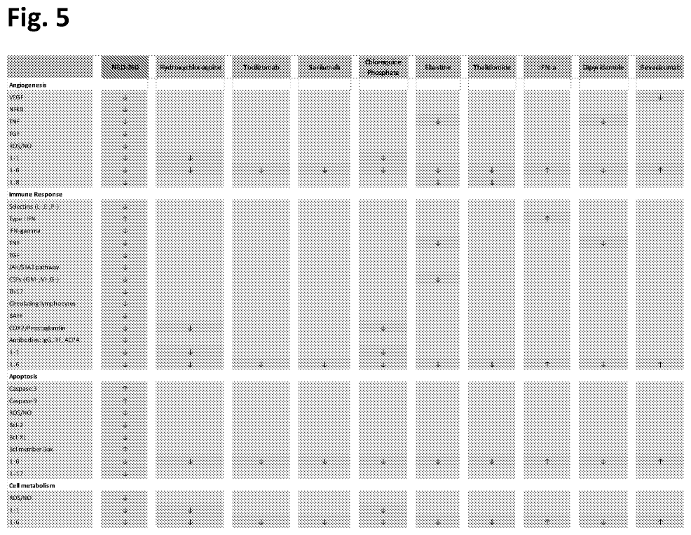

FIG. 6 shows certain agents according to the present disclosure and their effects on essential viral infection pathways.

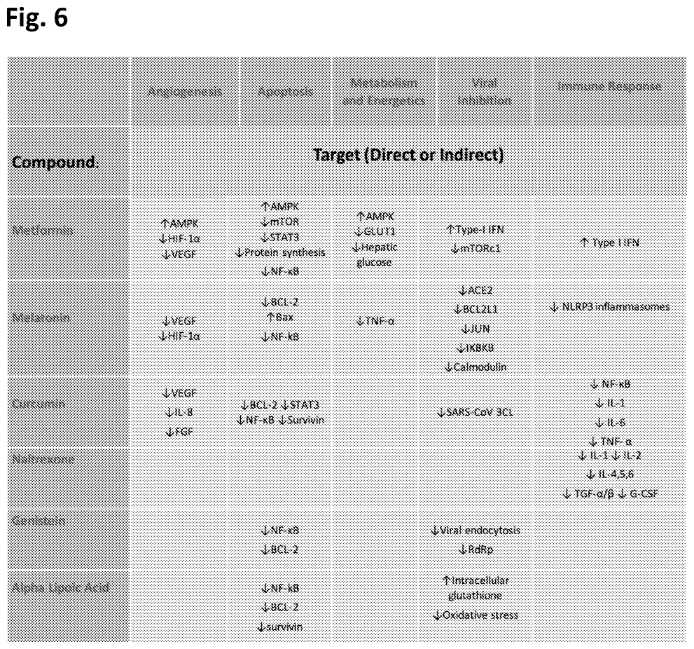

FIG. 7 shows certain MoAs for certain agents according to an embodiment of the present disclosure.

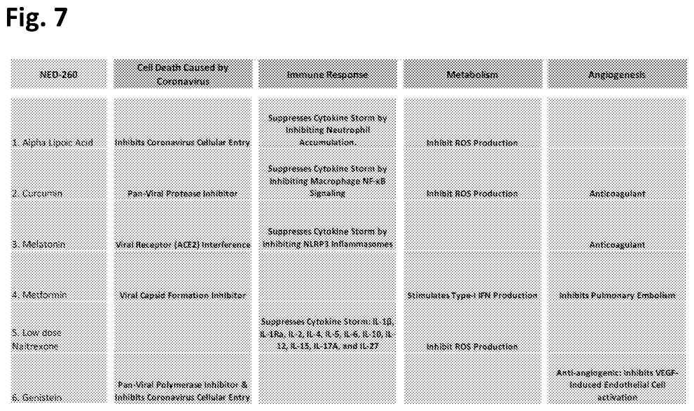

FIG. 8 shows certain the effects on stress and mental health for certain agents according to an embodiment of the present disclosure.

FIG. 9 shows exemplary cytotoxicity and viral neutralization activity data for several therapeutic compositions at varying dosages. Cytotoxicity and/or pseudovirus neutralization is shown for dilutions of (A) an embodiment of NED-260 (TA1); (B) remdesivir (TA2); and (C) SARS-CoV-2 spike antibody for positive control.

FIG. 10 shows exemplary pseudovirus neutralization activity data for several therapeutic compositions at varying dosages. Pseudovirus neutralization is shown for dilutions of (A) an embodiment of NED-260 (TA1); (B) remdesivir (TA2); and (C) SARS-CoV-2 spike antibody for positive control.

FIG. 11 shows exemplary plaque reduction assay (PRA) data for an embodiment of NED-260 at varying concentrations.

### DEFINITIONS

Below are provided certain definition of terms used herein, many or most of which confirm common understandings of those skilled in the art.

Activating agent: As used herein, the term “activating agent” refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control).

Administration: As used herein, the term “administration” typically refers to the administration of a composition to a subject or system to achieve delivery of an agent to the subject or system. In some embodiments, the agent is, or is included in, the composition; in some embodiments, the agent is generated through metabolism of the composition or one or more components thereof. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In many embodiments provided by the present disclosure, administration is oral administration. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Addressed: By “addressed”, when used in reference to a process or pathway targeted by therapy as described herein is meant that the process or pathway will be altered by the administration of an inventive therapeutic protocol (e.g., by one or a combination of agents included in an inventive therapeutic protocol) toward normalcy, that is, toward the characteristic function of that process or pathway in a normal individual, or an individual that does not suffer from a viral infection being treated, or disease or condition associated with a viral infection being treated.

Adult: As used herein, the term “adult” refers to a human eighteen years of age or older. Body weights among adults can vary widely with a typical range being 90 pounds to 250 pounds.

Agent: As will be clear to those skilled in the art, the term “agent” as used herein may refer to a compound or entity of any chemical class including, for example, polypeptides, nucleic acids, saccharides, lipids, small molecules, metals, or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Antagonist: As used herein, the term “antagonist” refers to an agent that i) inhibits, decreases or reduces the effects of another agent; and/or ii) inhibits, decreases, reduces, or delays one or more biological events. Antagonists may be or include agents of any chemical class including, for example, small molecules, polypeptides, nucleic acids, carbohydrates, lipids, metals, and/or any other entity that shows the relevant inhibitory activity. An antagonist may be direct (in which case it exerts its influence directly upon its target) or indirect (in which case it exerts its influence by other than binding to its target; e.g., by interacting with a regulator of the target, for example so that level or activity of the target is altered).

Antibody: As is known in the art, an “antibody” is an immunoglobulin that binds specifically to a particular antigen. The term encompasses immunoglobulins that are naturally produced in that they are generated by an organism reacting to the antigen, and also those that are synthetically produced or engineered. An antibody may be monoclonal or polyclonal. An antibody may be a member of any immunoglobulin class, including any of the human classes: IgG, IgM, IgA, and IgD. A typical immunoglobulin (antibody) structural unit as understood in the art, is known to comprise a tetramer. Each tetramer is composed of two identical pairs of polypeptide chains, each pair having one “light” (approximately 25 kD) and one “heavy” chain (approximately 50-70 kD). In some embodiments, an antibody may be a purified antibody (for example, by immune-affinity chromatography). In some embodiments, an antibody may be a human antibody. In some embodiments, an antibody may be a humanized antibody (antibody from non-human species whose protein sequences have been modified to increase their similarity to antibody variants produced naturally in humans). In some embodiments, an antibody may be a chimeric antibody (antibody made by combining genetic material from a non-human source, e.g., mouse, rat, horse, or pig, with genetic material from humans).

Antiviral agent: As used herein, the term “antiviral agent” refers to a class of medication used specifically for treating viral infections by inhibiting, deactivating, or destroying viral particles and/or components. In general, an antiviral agent may be or comprises a compound of any chemical class (e.g., a small molecule, metal, nucleic acid, polypeptide, lipid and/or carbohydrate). In some embodiments, an antiviral agent is or comprises an antibody or antibody mimic. In some embodiments, an antiviral agent is or comprises a nucleic acid agent (e.g., an antisense oligonucleotide, a siRNA, a shRNA, etc.) or mimic thereof. In some embodiments, an antiviral agent is or comprises a small molecule. In some embodiments, an antiviral agent is or comprises a naturally-occurring compound (e.g., small molecule). In some embodiments, an antiviral agent has a chemical structure that is generated and/or modified by the hand of man.

Approximately: As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Associated with: Two events or entities are “associated” with one another, as that term is used herein, if the presence, level and/or form of one is correlated with that of the other. For example, a particular entity (e.g., polypeptide, virus, etc.) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

Comparable: The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: A “composition” or a “pharmaceutical composition” according to this disclosure, refers to the combination of two or more agents as described herein for co-administration or administration as part of the same regimen. It is not required in all embodiments that the combination of agents result in physical admixture, that is, administration as separate co-agents each of the components of the composition is possible; however many patients or practitioners in the field may find it advantageous to prepare a composition that is an admixture of two or more of the ingredients in a pharmaceutically acceptable carrier, diluent, or excipient, making it possible to administer the component ingredients of the combination at the same time.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: Many methodologies described herein include a step of “determining.” Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form: As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Metronomic therapy: As used herein, the term “metronomic therapy” or “metronomic chemotherapy” refers to the frequent, e.g., daily, administration of therapeutic agents at doses significantly less than the maximum tolerated dose (MTD). For example, metronomic administration of Cyclophosphamide at a low dose, e.g., 50 mg/day as compared with representative MTD doses of 600 mg/m2-750 mg/m2 for three weeks, has shown promising results in a wide range of cancers. N. Penel et al., Critical Reviews in Oncology/Hematology, 82:40-50 (2012).

Modulator: The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Patient: As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, a patient is suffering from a viral infection. In some embodiments, the patient is suffering from a disease or condition associated with a viral infection.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable salt: As used herein, the term “pharmaceutically acceptable salt” means a salt prepared by conventional means, and are well known by those skilled in the art. Suitable pharmaceutically acceptable salts include, but are not limited to, salts of pharmaceutically acceptable inorganic acids included but not limited to hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic, ammonium, tetaalkylammonium, and valeric acids and the like. General information on types of pharmaceutically acceptable salts and their formation is known to those skilled in the art and is as described in general texts such as “Handbook of Pharmaceutical Salts” P. H. Stahl, C. G. Wermuth, 1st edition, 2002, Wiley-VCH.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Reference: The term “reference” is often used herein to describe a standard or control agent or value against which an agent or value of interest is compared. In some embodiments, a reference agent is tested and/or a reference value is determined substantially simultaneously with the testing or determination of the agent or value of interest. In some embodiments, a reference agent or value is a historical reference, optionally embodied in a tangible medium. Typically, as would be understood by those skilled in the art, a reference agent or value is determined or characterized under conditions comparable to those utilized to determine or characterize the agent or value of interest.

Refractory: The term “refractory” as used herein, refers to any subject or condition that does not respond with an expected clinical efficacy following the administration of provided compositions as normally observed by practicing medical personnel.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a viral infection response. Viral infection or subject response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of viral markers in a sample obtained from a subject, cytology, and/or histology. In particular, improvements in oxygen saturation rates, improvement in symptomatic shortness of breath, improvements in inflammatory markers, and improvement in white blood cell count can be used for assessing response. Many of these techniques attempt to determine the total viral infection burden. The exact response criteria can be selected in any appropriate manner, provided that when comparing groups of patients, the groups to be compared are assessed based on the same or comparable criteria for determining response rate. One of ordinary skill in the art will be able to select appropriate criteria.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic.

Solid form: As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: An individual who is “susceptible to” a disease, disorder, or condition (e.g., influenza) is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from the disease, disorder, or condition). In some embodiments, a risk of developing a disease, disorder, and/or condition is an age-based risk. In some embodiments, an individual who is susceptible to a disease, disorder, or condition is an individual who has been previously diagnosed with a different disease, disorder, and/or condition.

Symptoms are reduced: According to the present disclosure, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. Many patients that are virus carriers have no symptoms. It is not intended that the present disclosure be limited only to cases where the symptoms are eliminated. In some embodiments, the patient is administered present disclosure such that one or more symptoms is/are reduced (and the condition of the subject is thereby “improved”), albeit not completely eliminated.

Therapeutic agent: As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Therapeutic index: As is known in the art, the term “therapeutic index” refers to a ratio of unacceptably unsafe dose to efficacious dose for a particular index. Specifically, the therapeutic index is the ratio of TD50 (Dose that causes a toxic response in 50% of the relevant population) and ED50 (dose that is therapeutically effective in 50% of the population).

Therapeutic regimen: A “therapeutic regimen”, as that term is used herein, refers to a dosing regimen whose administration across a relevant population is correlated with a desired or beneficial therapeutic outcome.

Treatment: As used herein, the term “treatment” (also “treat” or “treating”) refers to any administration of a substance (e.g., antiviral agents) that partially or completely alleviates, ameliorates, relives, inhibits, delays onset of, reduces severity of, and/or reduces incidence of one or more symptoms, features, and/or causes of a particular disease, disorder, and/or condition (e.g., COVID-19). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### DETAILED DESCRIPTION OF CERTAIN EMBODIMENTS

The ensuing detailed description provides exemplary inventive embodiments, and the disclosure of specific examples is not intended to limit the scope, applicability, or configuration of inventions disclosed herein. It will be understood that various changes may be made to the specific combinations and/or arrangement of the elements or compounds without departing from the description or coverage of the claims. Substitution of known equivalents or specific equivalents disclosed herein, for example for any named component of a pharmaceutical composition or therapeutic regimen described in the application, is within the skill of practitioners in the relevant field.

In general, the present disclosure encompasses the recognition that many processes or pathways in an individual become altered in an individual suffering from a viral infection compared to the same processes or pathways as they function in normal individuals not suffering from a viral infection. The present disclosure further encompasses the recognition that pathways that become altered as a result of a viral infection, and in particular an RNA virus infection, overlap with certain pathways that advance all human cancers. In particular, the present disclosure identifies four pathways affected by viral infection (e.g., with an RNA virus) including immune response, apoptosis, metabolism, and angiogenesis.

As described herein, the present disclosure provides technologies for addressing multiple pathways involved in viral infection through use of combination therapies. In many embodiments, combinations of therapeutic agents are selected so that each pathway is addressed at least twice, and preferably three times, through use of the complete combination. By “addressed” is meant that the process or pathway will be altered by the administration of one or more of the composition components toward normalcy, that is, toward the characteristic function of that process or pathway in a normal individual, or an individual that does not suffer from the viral infection being treated. In some embodiments, combinations of therapeutic agents are selected so that processes or pathways are altered to treat or prevent diseases, disorders, and/or conditions that are associated with viral infection. In some embodiments, the combinations of therapeutic agents are selected and administered to treat or prevent secondary infections associated with a primary viral infection. In some embodiments, the combinations of therapeutic agents are selected and administered until both primary viral infection and secondary infections are addressed.

Among other things, the present disclosure recognizes that utilizing a combination of agents with particular mechanisms of action that influence pathways affected by viral infection (e.g., immune response, apoptosis, metabolism, and angiogenesis) can be advantageous over conventional antiviral therapies. In particular, the present disclosure identifies mechanisms of action including: (1) promoting shedding of viral receptors (e.g., ACE2) from the cell surface, which provides a prophylactic effect by preventing conventional viral entry; (2) inhibition of caveolae/clatherin-mediated endocytosis, which is a secondary route of viral entry; (3) crosslinking of viral (e.g., viral RNA) genome rendering it unable to express genes and replicate; (4) inhibition of viral protease (e.g., SARS-CoV 3C-like protease); (5) inhibition of viral RNA-dependent RNA polymerase; (6) inhibition of nucleocapsid protein (e.g., SARS-CoV nucleocapsid, N-protein); (7) increased production of IFN-α/β which can block SARS-CoV infection; (8) inhibition of ROS to reduce susceptibility to viral infections; and/or (9) inhibition of cytokines, e.g., IL-1 and IL-6, to protect against harmful immune responses (e.g., cytokine storm).

### Targeting Multiple Pathways

As discussed above, the present disclosure provides the teaching that effective anti-viral therapies utilize a combination of agents that together target multiple pathways that are crucial for viral infection, replication, and pathogenesis. In certain embodiments, a utilized combination includes a collection of agents that together address each targeted pathway at least twice. In some embodiments, at least one such pathway is targeted at least three times by a utilized combination. In some embodiments, each such pathway is targeted at least twice or at least three times by a utilized combination.

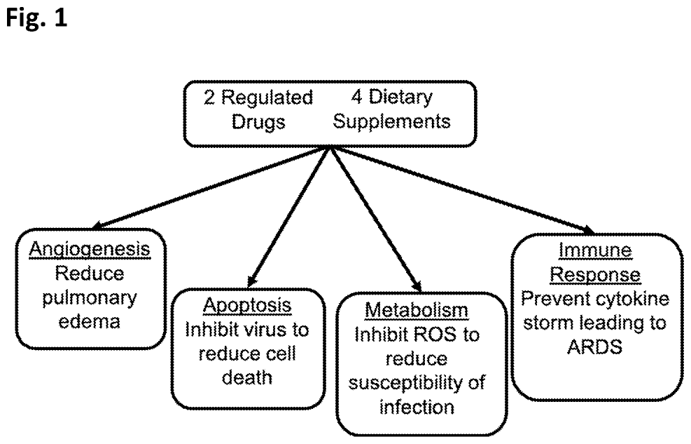

Those skilled in the art, reading the present disclosure and its identification of pathways to be targeted, as well as its teaching of assembling collections of agents that both target multiple pathways and, in some embodiments, target individual pathways more than once, will readily be able to select appropriate agents for combination use in accordance with the present disclosure. Below, pathways of particular interest for targeting in accordance with the present disclosure are discussed in further detail. A representative strategy for targeting each of these pathways is illustrated, for example, in FIG. 1.

Angiogenesis

“Angiogenesis” refers to the generation of new blood vessels into a tissue or organ. Under normal physiological conditions, humans or animals only undergo angiogenesis in very specific restricted situations. For example, angiogenesis is normally observed in wound healing, fetal and embryonal development, and formation of the corpus luteum, endometrium, and placenta. The endogenous control of angiogenesis is a highly regulated system of angiogenic stimulators and inhibitors. The control of angiogenesis has been found to be altered in certain disease states and, in many cases, the pathological damage associated with the disease is related to uncontrolled angiogenesis.

The present disclosure appreciates that angiogenic regulators in the human or animal body can generally be divided into two main groups: (1) pro-angiogenic regulators that directly or indirectly stimulate capillary and blood vessel growth, and (2) anti-angiogenic regulators or endogenous inhibitors that retard angiogenesis. Examples of pro-angiogenic regulators include, for example, Tumor Necrosis Factor (TNF-α), Granulocyte Colony-Stimulating Factor (GCSF), and Vascular Endothelial Growth Factor (VEGF). Examples of anti-angiogenic regulators include, for example, Interferon gamma (IFN-γ), Thrombospondin-1, and Angiostatin.

Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants (pro-angiogenic regulators) induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

In many diseases, including those associated with viral infection, angiogenesis is an important process that supports the disease, and therefore the process of angiogenesis itself becomes a target for therapeutic intervention. Recent medical research has documented the essential role angiogenesis plays in supporting disease. For example, in some viral infections, angiogenic factors, such as VEGFR2 and Ang2, are associated with the development conditions associated with lung damage such as pulmonary edema and ARDS and are predictive of mortality in patients suffering from such conditions. Further, it has been hypothesized, after studying the 2003 SARS-CoV outbreak, that binding of viral spike proteins to ACE2 on the surface of pneumocytes downregulates the receptor which allows ACE production of angiotensin II. Angiotensin II binding of type 1a angiotensin II receptor leads to pulmonary vascular permeability, suggesting that viral infection and subsequent lung injury may be due to vascular infiltration.

In some embodiments, the present disclosure utilizes an anti-angiogenic agent that has anticoagulant properties in combination with targeting of other hallmark pathways, as described herein. In some embodiments, the present disclosure utilizes curcumin as an anti-angiogenic agent that has anticoagulant properties. In some embodiments, the present disclosure utilizes melatonin as an anti-angiogenic agent that has anticoagulant properties. In some embodiments, the present disclosure utilizes melatonin and curcumin as anti-angiogenic agents that have anticoagulant properties.

In some embodiments, the present disclosure utilizes an anti-angiogenic agent that inhibits pulmonary embolism. In some embodiments, the present disclosure utilizes an anti-angiogenic agent that downregulates angiogenic factors (e.g., VEGFR2, Ang2, etc.) involved in pulmonary disorders (e.g., pulmonary edema, ARDS, etc.). In some embodiments, the present disclosure utilizes an anti-angiogenic agent that inhibits pulmonary embolism and downregulates angiogenic factors (e.g., VEGFR2, Ang2, etc.) involved in pulmonary disorders such as pulmonary edema, ARDS, and so forth. In some embodiments, the present disclosure utilizes Metfromin as an anti-angiogenic agent that inhibits pulmonary embolism and downregulates angiogenic factors (e.g., VEGFR2, Ang2, etc.) involved in pulmonary disorders such as pulmonary edema, ARDS, and so forth.

In some embodiments, the present disclosure utilizes an anti-angiogenic agent that inhibits VEGF-induced endothelial cell activation. In some embodiments, the present disclosure utilizes genistein as an agent that that inhibits VEGF-induced endothelial cell activation.

In some embodiments, the present disclosure utilizes metronomic anti-angiogenic therapy in combination with targeting of other hallmark pathways, as described herein. In some particular embodiments, the present disclosure utilizes metronomic anti-angiogenic therapy with an agent that also targets another pathway.

One example of a metronomic anti-angiogenic therapy, in some embodiments, the present disclosure utilizes metronomic cyclophosphamide for its anti-angiogenic effects, including, e.g., reduction of VEGF and endothelial cells, and upregulation of neovascularization inhibitor thrombospondin 1. The present disclosure recognizes that, in some instances, metronomic cyclophosphamide can cause blood clotting in a subject. Such blood clotting may be an unwanted side-effect in some subjects, e.g., subjects that are prone to adverse effects from clotting due to preexisting conditions or a viral infection. As such, in some embodiments, a subject can be assessed for a level of blood clotting or a risk level of blood clotting prior to administration of metronomic cyclophosphamide.

Apoptosis

Normal mammalian cells have a variety of programmed cell death pathways, designed to induce cellular suicide or apoptosis when cells are damaged or exposed to sub-optimal conditions. Apoptotic cell death prevents accumulation of undesirable mutations and, unlike necrotic cell death, eliminates cells without causing local tissue damage.

The present disclosure encompasses the recognition that TNF-α is a extrinsic mediator of apoptosis. Most cells in the human body have two receptors for TNF-α: TNFR1 and TNFR2. The binding of TNF-α to TNFR1 has been shown to initiate the pathway that leads to caspase activation via the intermediate membrane proteins TNF receptor-associated death domain (TRADD) and Fas-associated death domain protein (FADD). cIAP1/2 can inhibit TNF-α signaling by binding to TRAF2. FLIP inhibits the activation of caspase-8. Binding of this receptor can also indirectly lead to the activation of transcription factors involved in cell survival and inflammatory responses. However, signaling through TNFR1 might also induce apoptosis in a caspase-independent manner. The link between TNF-α and apoptosis shows why an abnormal production of TNF-α plays a fundamental role in several human diseases, especially in autoimmune diseases. The TNF-α receptor superfamily also includes death receptors (DRs), such as DR4 and DR5. These receptors bind to the protein TRAIL and mediate apoptosis.

The present disclosure further recognizes that two main hypotheses that link apoptosis with the pathogenesis of acute respiratory distress syndrome (ARDS) have been postulated, namely the “neutrophilic hypothesis” and the “epithelial hypothesis.” The former hypothesis suggests that neutrophil apoptosis plays an important role in the resolution of inflammation, and predicts that inhibition of neutrophil apoptosis or inhibition of clearance of apoptotic neutrophils is deleterious in ARDS. The epithelial hypothesis suggests that the epithelial injury seen during ARDS is associated with apoptotic death of alveolar epithelial cells in response to soluble mediators such as soluble Fas ligand, and predicts that blockade of such inhibitors may be beneficial in preventing or treating ARDS. These two hypotheses are not mutually exclusive, and both could play an important role in the pathogenesis of ARDS.

Studies in humans have shown that bronchoalveolar lavage (BAL) fluids from patients with early ARDS inhibit the rate at which neutrophils develop apoptosis in vitro. This inhibitory effect disappears at later stages of ARDS, as inflammation resolves. The inhibitory effect of BAL fluids on neutrophil apoptosis is mediated by soluble factors, primarily the pro-inflammatory cytokines granulocyte colony-stimulating factor and granulocyte/macrophage colony-stimulating factor (GM-CSF), and possibly IL-8 and IL-2. Clearance of apoptotic cells by phagocytes also plays a role in survival and persistence of inflammation during acute lung injury. Macrophages and other phagocytic cells recognize apoptotic cells via a number of membrane surface molecules. One of these membrane molecules, namely CD44, appears to play an important role in the clearance of apoptotic neutrophils in vivo and in vitro. Failure to clear apoptotic neutrophils has been associated with worsened inflammation and increased mortality. Further, phagocytosis of apoptotic neutrophils by macrophages inhibits macrophage production of pro-inflammatory cytokines (i.e. IL-1β, IL-8, IL-10, GM-CSF, and TNF-α) and increases release of anti-inflammatory mediators (i.e. transforming growth factor-β1, prostaglandin E2, and platelet-activating factor). Among other things, the present disclosure encompasses the recognition that increases in phagocytosis of apoptotic neutrophils could favor resolution of inflammation by down-regulating the inflammatory phenotype in activated alveolar macrophages.

In addition to neutrophil alveolitis, main features of ARDS typically include destruction of the alveolar epithelium, with severe damage to the alveolar capillary barrier and major increases in alveolar capillary permeability. The alveolar epithelium of patients who die from lung injury contains cells that exhibit evidence of DNA fragmentation, and alveolar pneumocytes from humans with diffuse alveolar damage show upregulation of Bax, a Bcl-2 analog that favors apoptosis. Evidence of extensive alveolar epithelial cell apoptosis has been described in murine models of pulmonary fibrosis and lipopolysaccharide-induced lung injury. Apoptosis of alveolar epithelial cells is detectable in mice as early as 6 hours after intratracheal administration of lipopolysaccharide.

The present disclosure identifies that anti-apoptotic agents could be particularly useful for the treatment of disease or disorders associated with viral infection. In some embodiments, anti-apoptotic agents are used for the treatment and/or prevention of one or more conditions (e.g., ARDS, or virus associated respiratory failure) associated with lung damage and resulting from viral infection. In some embodiments, the present disclosure utilizes anti-apoptotic agents in combination with targeting of other hallmark pathways, as described herein. In particular, the present disclosure recognizes that upon cellular entry of certain RNA viruses (e.g., SARS-CoV-2), TNF-α production is stimulated, metalloproteases are activated, and apoptosis can occur leading to lung damage (e.g., ARDS, or virus associated respiratory failure) and disease progression. In some embodiments, the present disclosure utilizes an anti-apoptotic agent that inhibits virus cellular entry. In some embodiments, the present disclosure utilizes alpha lipoic acid as an anti-apoptotic agent that inhibits virus cellular entry. In some embodiments, the present disclosure utilizes an anti-apoptotic agent that inhibits virus cellular entry via viral receptor interference. In some embodiments, the present disclosure utilizes melatonin as an anti-apoptotic agent that inhibits virus cellular entry via viral receptor interference.

In some embodiments of the disclosure an anti-apoptotic agent is utilized that inhibits viral replication after viral entry into a cell. In some embodiments, the present disclosure utilizes an anti-apoptotic agent that has pan-viral protease inhibitor activity. In some embodiments, the present disclosure utilizes curcumin as an anti-apopotic agent that has pan-viral protease inhibitor activity. In some embodiments, the present disclosure utilizes an anti-apopotic agent that has viral capsid formation inhibitor activity. In some embodiments, the present disclosure utilizes metformin as an anti-apoptotic agent that has viral capsid formation inhibitor activity.

Metabolism

The mitochondrion is the central organelle that regulates the metabolism of macromolecules, including carbohydrates, amino acids, and fatty acids. Glucose, which is the major source of energy, is converted to pyruvate in the cytoplasm via glycolysis. Under normal conditions in most cells, pyruvate is shuttled into mitochondria, where it is oxidized via the tricarboxylic acid (TCA) cycle, eventually generating ATP through the electron transport chain of the mitochondria in a process called oxidative phosphorylation. Mitochondria are highly prone to various cellular stress conditions and undergo damage and dysfunction leading to disruption of vital mitochondrial functions. Owing to their multifaceted role in myriad cellular functions, the maintenance of mitochondrial homeostasis is integral aspect of cellular stress response and homeostasis. Among other things, the present disclosure recognizes that virus infection, e.g., RNA virus infection, can directly or indirectly impair mitochondrial function and dynamics.

The present disclosure recognizes that during viral infection host cells trigger antiviral defense responses such as shutting down of translation, foreign RNA editing and degradation, interferon production, and so forth. Nevertheless, viruses have evolved strategies to escape or evade the host defense system in favor of viral propagation. The present disclosure appreciates that, in case of most RNA viruses, the cytosolic pathogen recognition receptors (i.e., RIG-I and MDA5) recognize viral RNAs and undergo conformational change and oligomerization thereby transducing the signal to the downstream signaling partner MAVS, an antiviral adaptor protein tethered to the OMM and mitochondria-associated membranes (MAM). Activated MAVS then coordinates the assembly of multimeric signaling complex called MAVS signalosome by facilitating recruitment of other host proteins (e.g., TRAFs, TBK1, and IRFs). The present disclosure particularly encompasses the recognition that the MAVS signalosome generates a highly cooperative context dependent signal resulting in the biogenesis of interferons (IFNs). Some viruses (e.g., hepatitis C virus) cleave the MAVS protein, thereby suppressing the host antiviral response, which represents one among many strategies exploited by the viruses to target mitochondria and evade host defense strategies.

The present disclosure recognizes that certain viruses evade the immune system by inhibiting mitochondrial antiviral signaling in order to suppress Type-I IFN production, allowing the virus to escape detection and elimination. The present disclosure further encompasses the recognition that it is desirable to treat viral infections by targeting metabolism and/or energetics pathways, particularly while targeting other pathways, as described herein. In some embodiments, among other things, the present disclosure utilizes metformin for its effects on metabolism, including targeting of the mitochondria respiratory chain complex I. Without wishing to be bound by theory, it is understood that metformin disrupts ATP production and ROS formation, and the subsequent down regulation of ATP increases the ratio of AMP:ATP that activates AMPK. Further, AMPK activation by metformin increases insulin sensitivity, and insulin sensitivity is associated with increased type-I interferon IFN-α/β signaling which leads to blocking of viral infection. In some embodiments, the present disclosure utilizes a metabolism modulating agent that stimulates Type-I interferon production. In some embodiments, the present disclosure utilizes metformin as a metabolism modulating agent that stimulates Type-I interferon production.

In some embodiments, the present disclosure utilizes a metabolism modulating agent to inhibit ROS production. In some embodiments, the present disclosure utilizes alpha lipoic acid as a metabolism modulating agent to inhibit ROS production. In some embodiments, the present disclosure utilizes curcumin as a metabolism modulating agent to inhibit ROS production. In some embodiments, the present disclosure utilizes naltrexone as a metabolism modulating agent to inhibit ROS production.

Immune Response

Cytokine release syndrome (CRS) or cytokine storm syndrome (CSS) is a form of systemic inflammatory response syndrome that can be triggered by a variety of factors such as infections and certain drugs. It occurs when large numbers of white blood cells are activated and release inflammatory cytokines, which in turn activate yet more white blood cells. CRS is also an adverse effect of some monoclonal antibody drugs, as well as adoptive T-cell therapies. Severe CRS or cytokine reactions can also occur in a number of infectious and non-infectious diseases including graft-versus-host disease (GVHD), coronavirus disease 2019 (COVID-19), acute respiratory distress syndrome (ARDS), sepsis, Ebola, avian influenza, smallpox, and systemic inflammatory response syndrome (SIRS). Although, severe acute respiratory syndrome coronavirus 2 (SARS-CoV-2) is sufficiently cleared by the early acute phase anti-viral response in most individuals, some progress to a hyperinflammatory condition, often with life-threatening pulmonary involvement. This systemic hyperinflammation results in inflammatory lymphocytic and monocytic infiltration of the lung and the heart, causing ARDS and cardiac failure. Patients with fulminant COVID-19 and respiratory failure (e.g., ARDS or other virus associated respiratory failure) have classical serum biomarkers of CRS including elevated CRP, LDH, IL-6, and ferritin.

The present disclosure recognizes that during certain viral infections, immune cells are hyperactivated, causing alveoli to be infiltrated, and triggering the cytokine storm/autoimmunity leading to inflammation and respiratory failure. The present disclosure further encompasses the recognition that it is desirable to treat viral infections by suppressing pathways that lead to cytokine storm. In some embodiments, the present disclosure utilizes an agent that suppresses cytokine storm by inhibiting neutrophil accumulation. In some embodiments, an agent that suppresses cytokine storm by inhibiting neutrophil accumulation is alpha lipoic acid. In some embodiments, the present disclosure utilizes an agent that suppresses cytokine storm by inhibiting macrophage NF-kB signaling. In some embodiments, an agent that suppresses cytokine storm by inhibiting macrophage NF-kB signaling is curcumin. In some embodiments, the present disclosure utilizes an agent that suppresses cytokine storm by inhibiting NLRP3 inflammasomes. In some embodiments, an agent that suppresses cytokine storm by inhibiting NLRP3 inflammasomes is melatonin. In some embodiments, the present disclosure utilizes an agent that suppresses cytokine storm by reducing plasma concentrations of interleukin (IL)-1β, IL-1Ra, IL-2, IL-4, IL-5, IL-6, IL-10, IL-12p40, IL-12p70, IL-15, IL-17A, IL-27, transforming growth factor (TGF)-α, TGF-β, tumor necrosis factor (TNF)-α, and granulocyte-colony stimulating factor (G-CSF).

### Anti-Viral Therapy

The present disclosure, among other things, identifies that a treatment that is collectively administered at safe doses and which targets multiple (e.g., at least four) core pathophysiological axes which are relevant to the progression of cancers: immune response, apoptosis, metabolism, and angiogenesis (the “hallmark” pathways of cancer), can also be utilized for the treatment and prevention of certain RNA virus infections. The present disclosure encompasses the recognition that these same four process are closely intertwined with the pathological features of certain viruses, e.g., RNA viruses, e.g., SARS-CoV-2. In particular, the present disclosure encompasses the recognition that certain combinations disclosed in International Publication WO 2014/169221A2 (the contents of which are hereby incorporated by reference) can be effective in the treatment and prevention of certain RNA virus infections.

The present disclosure provides new and improved strategies for developing and/or implementing RNA virus therapies. Among other things, the present disclosure appreciates the benefits of targeting multiple pathways, and furthermore appreciates that combinations of particular interest both target multiple pathways and target individual pathways in multiple ways.

Moreover, the present disclosure encompasses the recognition that in certain embodiments it is desirable to utilize agents (and/or dosing regimens) with a broader therapeutic index than that commonly observed for conventional chemotherapeutic agents and/or for many antivirals. Such conventional agents are typically characterized by a therapeutic index within the range of about 2 to about 5. In some embodiments, the present disclosure utilizes one or more agents whose therapeutic index is within the range of about 10 to about 100.

The present disclosure encompasses the particular insight that therapeutic agents developed for and/or effective in treatment of certain non-life-threatening conditions, and particularly of chronic conditions, may target one or more pathways that, as described herein, are essential pathways for RNA virus infection, and may be useful in combination therapies as described herein. Such agents typically show a wide safety margin, particularly when developed for long-term therapy. Indeed, agents approved for long-term therapy of non-life-threatening diseases, disorders, or conditions typically have had to meet stringent regulatory risk-benefit requirements. The lower the morbidity associated with the condition being treated, the lower the acceptable risk for its therapy. In accordance with certain embodiments of the present disclosure, agents developed for treatment of low-morbidity, chronic illnesses that target one or more pathways that are essential for viral, e.g., RNA viral, infection as described herein are particularly useful in inventive therapeutic regimens for the treatment of a virus infection.

Still further, the present disclosure encompasses the recognition that certain traditional and/or nutraceutical medicine approaches, including complementary and alternative medicines (CAM) identify and/or utilize well-tolerated agents that target the relevant pathways described herein. In many embodiments, such agents may be or include purified or partially purified natural products or extracts. In some embodiments, such products have been identified and/or characterized as a result of decades, or even centuries, of observational trial and error. Typically, traditional and/or nutraceutical agents are well tolerated (i.e., are associated with minimal toxicities), and show high therapeutic indices (e.g., typically well above 10, and often within the at least 10 to at least 100 range described herein, sometimes even higher).

Among other things, therefore, the present disclosure provides viral therapies that utilize combinations of agents that show high therapeutic indices and/or together target multiple essential pathways in virus infection, preferably in multiple ways.

In some embodiments, natural compounds and supplements referred to as “Nutraceuticals,” “Natural Medicines,” or “Phytomedicines” are suitable for use in combination therapies disclosed herein. As described herein, such nutraceuticals may be selected based on the quality and number of preclinical or clinical studies presenting either credible evidence of clinical anti-viral activity or demonstrating their ability to affect one or more pathways involved in viral infection described herein.

Exemplary agents useful for inclusion in certain embodiments of inventive combination therapy regimens for the treatment of RNA virus infection are discussed individually below. For many of them, there are a number of equivalent agents that will be known to those skilled in the art.

### Agents

Alpha Lipoic Acid

Alpha-lipoic acid is a fatty acid produced by the body for converting glucose into energy. It is also known to have antioxidant properties beneficial for fighting harmful chemicals that contribute to onset of disease. It is also referred to by the following names: Acetate Replacing Factor, A-Lipoic Acid, Acide Alpha-Lipoïque, Acide Alpha-Lipoïque R, Acide DL-Alpha-Lipoïque, Acide Lipoïque, Acide Thioctique, Acide 1,2-dithiolane-3-pentanoïque, Acide 1,2-dithiolane-3-valérique, Acide 5 Valérique (1,2-dithiolan-3-yl), Acide 6,8-dithiooctanoïque, Acide 6,8-Thioctique, Acido Alfa Lipoico, Alpha-Lipoic Acid Extract, ALA, Biletan, Extrait d'acide Alpha-Lipoïque, Lipoic Acid, Lipoicin, R-ALA, R-Alpha-Lipoic Acid R, S-Alpha Lipoic Acid, (R)-Lipoic Acid, R-Lipoic Acid, RS-Alpha-Lipoic Acid Thioctacid, Thioctan, Thioctic Acid, 1,2-dithiolane-3-pentanoic acid, 1,2-dithiolane-3-valeric acid, 6,8-dithiooctanoic acid, 6,8-thioctic acid, 5-(1,2-dithiolan-3-yl) valeric acid.

Although manufactured by the body and found in trace amounts in foods such as spinach, broccoli, peas, Brewer's yeast, brussel sprouts, rice, bran, potatoes and organ meats (kidney, heart, liver), it is the concentrated amounts of Alpha-Lipoic Acid found in supplements that provides the best antioxidant effect. When produced endogenously in plants or humans, it is complexed with proteins. However, when taken in supplement form, it is not bound to proteins and is likely in a 1000 fold greater amount than can be obtained through regular diet.

Among other things, the present disclosure recognizes that alpha-lipoic acid can inhibit TNF-α-induced NF-κB pathway activation which leads to endothelial activation and monocyte adhesion, which are the initial steps to leading to inflammation caused by oxidative stress. Alpha-lipoic acid has also been found to inhibit copper- and iron-mediated oxidative damage and accumulation via chelation of free metal ions. This process suppresses the induced oxidative damage caused by reactions that produce reactive free radicals. The addition of alpha-lipoic acid to cultured cells has been shown to activate PKB/Akt-dependent signaling resulting in increased survival of neurons.

Several alpha-lipoic acid supplements are presently manufactured. It is important to note that alpha-lipoic acid contains an asymmetric carbon, meaning there are two possible optical isomers that are mirror images of each other (R- and S-isomers). Most supplements may contain a 50/50 racemic mixture of each R-alpha-lipoic acid and S-alpha-lipoic acid. Supplements that contain only the R-isomer are available but the level of purity may be uncertain. Since taking alpha-lipoic acid with a meal decreases its bioavailability, it is generally recommended that it be taken on an empty stomach (one hour before or two hours after eating).

Commercial suppliers for alpha-lipoic acid include Source Naturals Alpha Lipoic Acid, Swanson Ultra Alpha Lipoic Acid, NOW Foods Alpha Lipoic Acid, Bluebonnet Alpha Lipoic Acid, Country Life R-Lipoic Acid, Solgar Alpha Lipoic Acid.

Alpha lipoic acid is a versatile agent with the capacity to enact many potentially protective effects for patients who have, or are at risk of, exposure to SARS-CoV-2 by managing co-morbidities that aggravate the ramifications of infection, exerting direct antiviral effects, and also mitigating the harm of an aggressive inflammatory response following infection.

Indeed, it is now well known that patients who present with comorbid chronic conditions such as diabetes, cardiovascular disease, and cancer are at an increased risk of suffering severe complications from COVID-19, whether through the infection itself or exacerbation of their pre-existing conditions. The present disclosure encompasses the recognition that there is utility in employing alpha lipoic acid for at-risk patients who suffer from diabetic or pre-diabetic conditions, which constitute a large portion of the population. According to the 2020 CDC National Diabetes Statistics Report, >100M Americans have at least pre-diabetes, and nearly 10% have diabetes. Alpha lipoic acid, along with its enzymatically reduced form dihydrolipoic acid, function to ameliorate diabetes by serving as strong antioxidants that can mimic insulin and exert anti-inflammatory effects. Alpha lipoic acid can do so by acting as a metal chelator, regenerating other endogenous antioxidants, and even regulating gene expression in pathways that enhance anti-inflammatory phenotypes.

Alpha lipoic acid has further been tested in vitro on the MDCK cell line, which is often used as a general model for epithelial cells, during which it was shown to significantly reduce replication and propagation of RNA viruses similar to SARS-CoV-2. More specifically with regard to human coronaviruses, alpha lipoic acid has also been shown to mediate protective effects by way of its ability to increase levels of intracellular glutathione and regulate oxidative stress. In host cells treated with the human coronavirus 229E, it was identified that intracellular oxidative stress was a major factor influencing infectivity, and that administration of alpha lipoic acid was actually capable of reducing susceptibility to coronavirus infection.

Finally, patients who are critically ill often suffer from severe inflammatory response to the virus, including the cytokine storm, and exhibit rates of neutrophil infiltration in the lungs, which generates pneumonia and many of the respiratory issues that leads to patient illness and death. Alpha lipoic acid, as an inflammatory agent, has demonstrated capacity in other dysregulated physiological contexts to suppress neutrophil accumulation. Indeed, in an in vivo model of gut inflammation, intraperitoneal and oral administration of alpha lipoic acid significantly reduced neutrophil load and presence of reactive oxygen species.

In many embodiments of the present disclosure, alpha lipoic acid is administered in a dosing regimen that is or includes doses of at least 600 mg/day to 2400 mg/day. In some embodiments, alpha-lipoic acid is administered in a dosing regimen that is or includes doses of at least 600 mg/day to 1200 mg/day. In certain particular embodiments, alpha-lipoic acid is administered in a dosing regimen that is or includes doses of 1200 mg/day.

In some embodiments, a combination comprises alpha lipoic acid at a dose of about 600-2400 mg/day. In some embodiments, a combination comprises alpha lipoic acid at a dose of at least 500 mg/day, at least 600 mg/day, at least 700 mg/day, at least 800 mg/day, at least 900 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, at least 1800 mg/day, at least 1900 mg/day, at least 2000 mg/day, at least 2100 mg/day, at least 2200 mg/day, or at least 2300 mg/day. In some embodiments, a combination comprises alpha lipoic acid at a dose of at most 2700 mg/day, at most 2600 mg/day, at most 2500 mg/day, at most 2400 mg/day, at most 2300 mg/day, at most 2200 mg/day, at most 2100 mg/day, at most 2000 mg/day, at most 1900 mg/day, at most 1800 mg/day, at most 1700 mg/day, at most 1600 mg/day, at most 1500 mg/day, at most 1400 mg/day, at most 1300 mg/day, at most 1200 mg/day, at most 1100 mg/day, at most 1000 mg/day, at most 900 mg/day, at most 800 mg/day, or at most 700 mg/day.

In some embodiments, a combination comprises alpha lipoic acid at a dose of about 1200 mg/day.

Curcumin

The active ingredient in the spice Turmeric is curcumin, which is extracted from the rhizome of the plant Curcuma longa Linn. Curcumin is the principal curcuminoid, or polyphenolic compound found in such extracts, with others including demethoxycurcumin and bisdemethoxycurcumin.

Turmeric is also known as Curcuma, Curcuma aromatica, Curcuma domestica, Curcumae longa, Curcumae Longae Rhizoma, Curcumin, Curcumine, Curcuminoid, Curcuminoide, Curcuminoides, Curcuminoids, Halada, Haldi, Haridra, Indian Saffron, Nisha, Pian Jiang Huang, Racine de Curcuma, Radix Curcumae, Rajani, Rhizoma Cucurmae Longae, Safran Bourbon, Safran de Batallita, Safran des Indes, Turmeric Root, Yu Jin.

Curcumin's mechanisms of action include inhibition of several cell signaling pathways, effects on cellular enzymes such as cyclooxygenase and effects on angiogenesis and cell-cell adhesion. Curcumin also affects gene transcription and induces apoptosis.

Curcumin is effective at inhibiting the signal transduction pathway of PI3K/Akt, MAPK, and NF-wB activation, as well as the Sonic Hedgehog (Shh) signaling pathway by down-regulating the Shh protein. In turn, reduction of beta-catenin, the activated/phosphorylated form of Akt and NF-wB, triggers apoptosis.

Pathways associated with oncogenesis that are inhibited by curcumin include down-regulation of epidermal growth factor receptors (EGFR and erbB2), Insulin-like growth factor type-1 receptor (IFG-1R), sonic hedgehog (SHH)/GLIs) and Wnt/b-catenin and PARP, IKK, EGFR, JNK, MAPK and 5-LOX. In addition curcumin suppresses downstream signaling elements such as signal transducers and activators of transcription (STATs), PI3K/Akt, nuclear factor-kappa B (NF-xB), and its targeted genes, including IL-6, COX-2, and MMPs.

Curcumin is most beneficial when take in liposomal form. The most bioavailable supplement is Life Extension's Super Bio Curcumin® which absorbs into the bloodstream up to seven times better than conventional 95% curcumin extract. Alternatively or additionally, another highly bioavailable form of Curcumin is Euromedica CuraPro BCM-95® or Progressive Labs Curcumin BCM-95®. Other curcumin supplements add piperine, (Piper nigrum) to enhance absorption of curcumin in their products. However, the interactions of piperine with many medications can cause problems including toxicity if taken in high doses. Curcumin can exist in the tautomeric forms that include the 1,3-diketo and the enol form. The most stable form of Curcumin is its planar enol form. Additionally Biomar™ Curcumin is commercially available.

Alternatives to Super Bio-Curcumin and/or Euromedica BCM-95® include all 95% Curcumin supplements including Jarrow Formulas Curcumin 95, NOW Foods Curcumin, Genceutic Naturals Curcumin BCM-95, etc.

Turmeric Extract in fact only provides 2-6% curcumin, and it can be important to take Curcumin in higher levels. Any supplement that is lower than 95% Curcumin is not as effective. Only about 50-60% is absorbed (in contrast to 96% absorption with Super Bio Curcumin®). In addition, dilutions with other supplements such as bioperine reduce bioavailability, and as mentioned piperine can interact with other medications negatively. Synthetic, petroleum-derived curcumin supplements may only contain on or two of the important curcuminoids found in natural supplements. Resveratrol is not a substitute. There are “Ultimate Antioxidants” that contain Curcumin and other important antioxidants, but do not reach the quality or bioavailability of other supplements (e.g. Natural Factors® Ultimate Antioxidant claims 95% total curcuminoids, but does not specify which, and has 13 other factors that may or may not be beneficial).

In some embodiments, when administering Curcumin, consideration may desirably be given to factors impacting a patient's ability to absorb administered material. For example, if may be desirable or necessary to reduce or eliminate one or more Curcumin otherwise desirable or appropriate Curcumin doses if a patient has suffered damage (including, for example, removal by surgery; see Examples) of part of his or her gastrointestinal tract. Alternatively or additionally, it might be desirable to administer Curcumin in a more palatable or bioavailable form (e.g., as a liquid) to certain patients.

The present disclosure encompasses the recognition that exposed patients with disease progression of certain RNA viral infections (e.g., SARS-CoV-2) will often face severe pneumonia, and as such, regulation of the processes that contribute to lung damage and failure would be a highly valuable preventive measure. In severe pneumonia caused by influenza, curcumin has already demonstrated beneficial effects in vitro and in vivo; it was found that curcumin was able to inhibit influenza in vitro and even mitigate severe symptoms in mice that were infected. Indeed, in infected mice treated with curcumin, there was reduced lung injury, reduced production of highly inflammatory cytokines, and inhibition of macrophage NF-xB signaling. In addition, in other severe viral infections, curcumin was similarly able to preclude cytokine storms, a symptom that often appears in the terminal stage of disease, by inhibiting IL-1, IL-6 and TNF-alpha.

Curcumin has also been proposed to exert antiviral effects on components of coronaviruses, in particular, by targeting the SARS-CoV 3CL protease which is involved in the viral replication process More generally in other studies, there has been evidence that curcumin mediates inhibitory effects on many stages of the viral life cycle, including attachment to cell surfaces, entry, activation of host cell programs, and envelopment for release.

By virtue of its anti-inflammatory, antioxidant, and anti-thrombotic effects, curcumin also has potential to prophylactically protect co-morbid patients who might suffer severe complications from RNA virus infection. In in vivo models, curcumin has been shown to reduce serum cholesterol and mitigate many pathological outcomes associated with plaque formation in the vasculature; it was also shown to reduce the onset of heart failure as well. Cardiac involvement as a byproduct of the SARS-CoV-2 infection has been demonstrated in a number of patients, including reports of myocarditis. Previous in vivo studies in mice have exhibited that curcumin can be protective of myocarditis via inhibition of the phosphatidylinositol 3 kinase/Akt/NF-wB pathway.

In many embodiments of the present disclosure, curcumin is administered in a dosing regimen that is or includes doses of at least 750 mg/day to 3000 mg/day. In some embodiments, curcumin is administered in a dosing regimen that is or includes doses of at least 1000 mg/day to 3000 mg/day. In certain particular embodiments, curcumin is administered in a dosing regimen that is or includes doses of 1500 mg/day.

In some embodiments, a combination comprises curcumin at a dose of about 200-3000 mg/day. In some embodiments, a combination comprises curcumin at a dose of at least 100 mg/day, at least 200 mg/day, at least 300 mg/day, at least 400 mg/day, at least 500 mg/day, at least 600 mg/day, at least 700 mg/day, at least 800 mg/day, at least 900 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, at least 1800 mg/day, at least 1900 mg/day, at least 2000 mg/day, at least 2100 mg/day, at least 2200 mg/day, at least 2300 mg/day, at least 2400 mg/day, or at least 2500 mg/day. In some embodiments, a combination comprises curcumin at a dose of at most 3000 mg/day, at most 2900 mg/day, at most 2800 mg/day, at most 2700 mg/day, at most 2600 mg/day, at most 2500 mg/day, at most 2400 mg/day, at most 2300 mg/day, at most 2200 mg/day, at most 2100 mg/day, at most 2000 mg/day, at most 1500 mg/day, at most 1400 mg/day, at most 1300 mg/day, at most 1200 mg/day, at most 1100 mg/day, at most 1000 mg/day, at most 900 mg/day at most 800 mg/day, at most 700 mg/day, at most 600 mg/day, at most 500 mg/day, at most 400 mg/day, or at most 300 mg/day.

In some embodiments, a combination comprises curcumin at a dose of about 1500 mg/day.

Melatonin

Melatonin is a hormone secreted by the pineal gland and found naturally in the body. Melatonin is also synthetically produced in a laboratory for medical use. It is also referred to as MEL, Melatonina, Mélatonine, MLT, N-acetyl-5-methoxytryptamine, N-Acétyl-5-Méthoxytryptamine, and Pineal Hormone.

Melatonin has known, potent anti-oxidant, anti-inflammatory, and anti-tumor properties, but it also influences oncogenic pathways including mTOR, which plays a role in pancreatic cancer. Melatonin induces pro-apoptotic signaling in pancreatic cancer cells; restores mitochondrial function which in turn restores apoptosis of pancreatic cancer cells; and enhances patients' responses to Capecitabine (XELODA). Leja-Szpak, et al., J Pineal Res., 49(3):248-55 (2010); Gonzalez, et al., J Pineal Res., Epub 2010; Ruiz-Rabelo, et al., Pancreas, Epub 2010.

Melatonin is known to suppress tumor angiogenesis by inhibiting HIF-1α stabilization under hypoxia, leading to a decrease in VEGF expression. Melatonin also inhibits cell proliferation and migration of HUVECs and also decreases both the VEGF protein secreted and the protein produced by pancreatic carcinoma cells. In addition, VEGF mRNA expression is known to be down-regulated by melatonin. Melatonin has also been shown to inhibit cell proliferation and induce apoptosis in cancer cells in vitro by simultaneously suppressing the COX-2/PGE2, p300/NF-κB, and PI3K/Akt/signaling and activating the Apaf-1/caspase-dependent apoptotic pathway.

The most beneficial form of Melatonin is in pharmaceutical grade (not “natural”, animal, or bovine) supplements having a purity of 99% or greater. The bioavailability of melatonin varies widely. A bioavailable source is Thorne Research Melatonin-5™. Melatonin has several clinical analogs that bind to melatonin receptors, but ultimately have a different function (most commonly as a sleep aid only or antidepressant only). These include S20242, agomelatine, and 2-Bromomelatonin. When melatonin, ramelteon, tasimelteon, PD-6735, and agomelatine are compared, agomelatine is the analogue that exhibits the most potential for the treatment of major depression. Unlike melatonin, agomelatine is a competitive antagonist of human and porcine serotonin (5-HT2C) receptors and human 5-HT2B receptors.

Alternatively or additionally, there are medications that include impurities and low levels of melatonin, for example, Circadin used for insomnia. Melatonin should only be taken in synthetic (man-made) form. The alternative that is extracted from ground-up cow pineal glands is rarely used, as it may spread disease.

Melatonin is reported to be useful in the treatment of a variety of diseases, disorders, and conditions, and recommended dosing regimens include, for example:

For age-related macular degeneration (vision loss with age), three milligrams of melatonin have been taken by mouth nightly at bedtime for six months.

To improve body temperature regulation in the elderly, 1.5 milligrams of melatonin has been taken by mouth nightly for two weeks.

For Alzheimer's disease or cognitive decline, melatonin has been taken by mouth in doses of 1-10 milligrams daily for 10 days up to 35 months.

For inflammation, melatonin has been taken by mouth in doses of 10 milligrams nightly for six months or five milligrams the night before and one hour before surgery.

For asthma, three milligrams of melatonin has been taken by mouth for four weeks.

For withdrawal from benzodiazepines (antianxiety agents), doses of 1-5 milligrams have been taken by mouth daily for from several weeks up to one year.

For cancer, melatonin has been taken by mouth in doses of 1-40 milligrams daily, with 20 milligrams being most common, for several weeks to months.

Melatonin has been applied to the skin.

For chronic fatigue syndrome, five milligrams of melatonin has been taken by mouth five hours before bed for three months.

For COPD (chronic lung disorder causing breathing difficulty), three milligrams of melatonin has been taken by mouth nightly two hours before bed for three months.

For circadian rhythm sleep disorders in people with and without vision problems, melatonin has been taken by mouth as a single dose of 0.5-5 milligrams before bed or as a daily dose for 1-3 months.

For delayed sleep phase syndrome, melatonin has been taken by mouth in doses of 0.3-6 milligrams, with five milligrams being most common, daily before sleeping for two weeks to three months.

For delirium, 0.5 milligrams of melatonin has been taken by mouth nightly for up to 14 days.

For depression, six milligrams of slow-release melatonin has been taken by mouth at bedtime for four weeks.

For exercise performance, 5-6 milligrams of melatonin has been taken by mouth one hour before exercise or before bedtime.

For fertility, three milligrams of melatonin has been taken by mouth nightly from the third to fifth day of the menstrual cycle until hormone injection (human chorionic gonadotropin, HCG), or on the day of hormone injection.

For fibromyalgia, 3-5 milligrams of melatonin has been taken by mouth nightly for four weeks to 60 days.

For stomach and intestine disorders, 3-10 milligrams of melatonin has been taken by mouth nightly for 2-12 weeks.

For headache, 2-10 milligrams of melatonin has been taken by mouth nightly for 14 days to eight weeks.

For liver inflammation, five milligrams of melatonin has been taken by mouth twice daily for 12 weeks.

For high blood pressure, melatonin has been taken by mouth in doses of 1-5 milligrams either as a single dose during the day or before bedtime, or daily for 1-3 months.

For high cholesterol, five milligrams of melatonin has been taken by mouth daily for two months.

For insomnia in the elderly, melatonin has been taken by mouth in doses of 0.1-5 milligrams at or two hours before bedtime for up to several months, in the form of melatonin-rich night milk or slow-release Circadin®. A dose of 0.5 milligrams has been placed in the cheek for four nights.

For jet lag, 0.1-8 milligrams of melatonin has been taken by mouth on the day of travel (close to target bedtime at destination), then daily for several days, in the form of standard or slow-release melatonin (Circadin®).

For memory, three milligrams of melatonin has been taken by mouth before testing.

For menopause, three milligrams of melatonin has been taken by mouth nightly at bedtime for 3-6 months.

For Parkinson's disease, doses of 3-50 milligrams have been taken by mouth nightly before bed for 2-10 weeks. (High doses of 3-6.6 grams of melatonin have also been taken by mouth daily; however, these doses were used in an older 1972 study and are no longer in use.)

For periodic limb movement disorder, three milligrams of melatonin has been taken by mouth nightly for six weeks.

For REM sleep behavior disorder, 3-12 milligrams of melatonin has been taken by mouth daily for four weeks.

For restless leg syndrome, a single dose of three milligrams of melatonin has been taken by mouth.

For sarcoidosis (chronic widespread inflammation), 20 milligrams of melatonin has been taken by mouth daily for one year, then decreased to 10 milligrams for a second year.

For muscle movement problems in people with schizophrenia, 2-10 milligrams of melatonin has been taken by mouth daily.

For seasonal affective disorder (SAD), two milligrams of sustained-release melatonin has been taken by mouth 1-2 hours nightly for three weeks. A dose of 0.5 milligrams of melatonin has been taken under the tongue for six days.

For seizure disorders, doses of melatonin taken by mouth were 3-10 milligrams daily for 2-4 weeks to three months.

For sleep (general), doses of melatonin taken by mouth were 0.3-10 milligrams.

For sleep disorders in people with behavioral, developmental, or mental disorders, 0.1-10 milligrams of melatonin has been taken by mouth daily for up to one year.

For sleep disturbance in Alzheimer's disease, 1.5-10 milligrams of melatonin has been taken by mouth nightly for 10 days to 35 months, together with light exposure or in the form of capsules.

For sleep disturbance in those with asthma, three milligrams of melatonin has been taken by mouth for four weeks.

For sleep disturbance in those with autism, 0.75-10 milligrams of melatonin has been taken nightly before bedtime for two weeks to six months.

For sleep disturbance in those with COPD, three milligrams of melatonin has been taken by mouth nightly.

For sleep disturbance in those with cystic fibrosis, three milligrams of melatonin has been taken by mouth nightly at bedtime for 21 days.

For sleep disturbance in those with depression, 0.5-10 milligrams of melatonin has been taken by mouth for 3-4 weeks, in addition to regular therapy.

For sleep disturbance in healthy people, 0.1-80 milligrams of melatonin has been taken by mouth, generally nightly before bed for one or several days up to 26 weeks. A dose of 50 milligrams has been injected into the vein.

For sleep disturbance in people undergoing hemodialysis, three milligrams of melatonin has been taken by mouth for six weeks.

For sleep disturbance in hospitalized and medically ill people, 3-5.4 milligrams of melatonin has been taken by mouth nightly.

For sleep disturbance in people with a learning disability, 0.5-9 milligrams of melatonin has been taken by mouth for 32-73 days.

For sleep disturbance in those with Parkinson's disease, 3-50 milligrams of melatonin has been taken by mouth at bedtime for 2-4 weeks.

For sleep disturbance after surgery, five milligrams of melatonin has been taken by mouth for three nights.

For sleep disturbance in people with mental disorders, 2-12 milligrams of melatonin has been taken by mouth daily before resting for up to 12 weeks

For sleep disturbance in people with traumatic brain injury, five milligrams of melatonin has been taken by mouth for one month.

For sleep disturbance in people with tuberous sclerosis complex (a genetic disorder causing tumors to grow in brain and other organs), five milligrams of melatonin has been taken 20 minutes before bed for two weeks.

For smoking, 0.3 milligrams of melatonin has been taken by mouth 3.5 hours after nicotine withdrawal.

For surgery, 3-15 milligrams of melatonin has been taken by mouth or placed under the tongue, and 0.05-0.2 milligrams per kilogram has been placed under the tongue, either alone or with other sedatives, typically 90 minutes before surgery or the night before and 90 minutes before surgery.

For anxiety or sedation before surgery, 3-10 milligrams and/or 0.05-0.5 milligrams per kilogram of melatonin have been injected into the vein, either alone or with other sedatives before surgery.

For tardive dyskinesia (uncontrolled, repetitive movements), 2-20 milligrams of melatonin has been taken by mouth for 4-12 weeks.

For low platelets, 20 milligrams of melatonin has been taken by mouth nightly for two months.

For ringing in the ears, three milligrams of melatonin has been taken by mouth daily for up to 80 days.

For ulcers, five milligrams of melatonin has been taken by mouth twice daily for 21 days together with other medications.

For nighttime urination, two milligrams of melatonin has been taken by mouth daily for four weeks.

For work shift sleep disorder, 1.8-10 milligrams of melatonin has been taken by mouth up to three times daily for up to six days before daytime sleep after a night shift.

For skin sun damage, melatonin has been applied to the skin in the form of a gel (20-100 milligrams of melatonin in 70% ethanol, in concentrations of 0.05-0.5% in 0.12 milliliters of gel); 0.6 milligrams per meter squared from 15 minutes before to 240 minutes after sun exposure, alone or with vitamins C and E; five percent melatonin in ethanol, propylene glycol, and water; and 5.85 microliters of solutions containing 1.2-5% melatonin, alone or with vitamins C and E.

Melatonin can impact on circadian rhythm differently depending on the time of day at which it is taken, so that attention is typically given to the timing of melatonin dosing.

In some embodiments, the present disclosure encompasses the recognition that melatonin promotes shedding of the ACE2 surface receptor required for the virus to enter cells. If used as a prophylactic, especially in individuals who have low endogenous production of melatonin which significantly decreases with age, melatonin could protect against viral infection by preventing or substantially lowering the efficacy with which the virus can enter cells of the respiratory system. If the virus does not enter cells, then the disease cannot manifest. Using network proximity analyses of drug targets and HCoV-host interactions in the human interactome, melatonin was identified as one of 16 potential anti-HCoV repurposable drugs that are further validated by enrichment analyses of drug-gene signatures and HCoV-induced transcriptomics data in human cell lines. Melatonin is an anti-inflammatory drug that indirectly targets several SARS-CoV-2 cellular targets, including ACE2, BCL2L1, JUN, and IKBKB. ACE2 is the receptor presented on the outer surface of cells, and it must interact with the S protein of SARS-CoV-2 in order for SARS-CoV-2 to gain entry into cells in order to replicate. In addition, melatonin inhibits NLRP3 inflammasomes, and this could significantly decrease the risk that a patient develops ARDS/ALI/other life-threatening complications. Melatonin has been shown to prevent ALI in mouse models.

The SARS-Cov-2 S protein must first be activated by ADAM17 then it can interact with ACE2 in order to achieve entry into the cell. After the virus enters the cell, it triggers the shedding of the ACE2 receptor to ensure that this cell is not infected by too many viral particles at once which could overburden and kill the cell before viral replication and release can conclude.

Crucially, melatonin was reported to inhibit calmodulin and calmodulin interacts with ACE2 by inhibiting shedding of the ACE2 ectodomain. Therefore, melatonin promotes shedding of the ACE2 receptor from the cell surface. Again, ACE2 is the protein on the outer surface of cells in the lungs and GI tract, and binding of ACE2 by the SARS-CoV-2 S protein mediates cellular entry of the virus. If melatonin prompts shedding of the ACE2 receptor before an individual is exposed to SARS-CoV-2, then SARS-CoV-2 is prevented, or at least substantially hindered from entering cells because cells will lack the surface receptor necessary for SARS-CoV-2 entry into cells. Hence, melatonin could provide a prophylactic effect preventing the virus from infecting individuals who take melatonin before being exposed to or testing positive for the virus.

Upon cellular entry of certain RNA viruses (e.g., SARS-CoV-2), TNF-α production is stimulated, metalloproteases are activated, and apoptosis can occur leading to lung damage (e.g., ARDS, or virus associated respiratory failure) and disease progression. Melatonin inhibits NLRP3 inflammasomes, and this, in combination with the promotion of ACE2 shedding, could significantly dampen viral titers and decrease the risk that a patient develops ARDS/ALI/other life-threatening complications if a patient begins taking melatonin after being exposed to or testing positive for the virus.

In accordance with embodiments of the present disclosure, suitable amounts of Melatonin will be 0.3-75 mg, preferably 1.0-50 mg, more preferably 1.0-20 mg, more preferably 1.0-10 mg, more preferably 2.0-10 mg per day. Most preferably the dosage amounts will range between 0.3 mg and 5.0 mg, between 1.0 mg and 5.0 mg, or between 3.0 mg and 6.0 mg, with all or part of the dose being administered at night/bedtime. Particularly preferred dosages will be 3.0-6.0 mg nightly, or 10-50 mg nightly in severe cases. In certain particular embodiments, melatonin is administered in a dosing regimen that is or includes doses of 10 mg/day.

In some embodiments, a combination comprises melatonin at a dose of about 1-40 mg/day. In some embodiments, a combination comprises melatonin at a dose of at least 0.5 mg/day, at least 1 mg/day, at least 5 mg/day, at least 10 mg/day, at least 15 mg/day, at least 20 mg/day, at least 25 mg/day, at least 30 mg/day, or at least 35 mg/day. In some embodiments, a combination comprises melatonin at a dose of at most 50 mg/day, at most 45 mg/day, at most 40 mg/day, at most 35 mg/day, at most 30 mg/day, at most 25 mg/day, at most 20 mg/day, at most 15 mg/day, at most 10 mg/day, or at most 5 mg/day.

In some embodiments, a combination comprises melatonin at a dose of about 10 mg/day.

Metformin

Metformin is a pharmaceutical compound initially indicated for diabetes and has the following brand names: Glucophage, Riomet®, Fortamet, Glumetza. Approved dosing regimens for diabetic patients are individually tailored, with maximum recommended daily dosages set at 2550 mg for adults or 2000 mg for pediatric patients. Typically, clinically significant responses are not seen at doses below 1500 mg/day. However, therapy is typically initiated with a lower starting dose (e.g., 500 mg once or twice/day or 85 mg/day), with gradually increasing subsequent doses (e.g., increasing in increments of 500 mg/week or 850 mg/2 weeks)

Metformin modulates the mTOR pathway, which antiproliferative effects during treatment with paclitaxel.

Metformin also functions in reducing cell growth, protein synthesis, MAPK3/1, and P90RSK phosphorylation in response to IGF1 through an AMPK-dependent mechanism in cultured bovine granulosa cells. In addition, Metformin strongly inhibited the proliferation, migration, and MMP-2 and -9 expression of HUVECs, also partially AMPK-dependent. Metformin also inhibits cell proliferation, migration and invasion through re-expression of miRNAs and decreased expression of CSC-specific genes.

Sources of Metformin include Metformin hydrochloride, which is a derivative of metformin present in Riomet (brand name analogs Apo-Metformin, Fortamet, Gen-Metformin, Glucophage, Glucophage XR, Glycon, Metformin HCL, Novo-Metformin, Nu-Metformin). Brand names of combination products include Actoplus Met (Metformin and pioglitazone), Avandamet (Metformin and rosiglitazone), Glucovance (Metformin and Glyburide), Janumet (Metformin and sitagliptin), Kombiglyze (Metformin and saxagliptin), Metaglip (Metformin and Glipizide), PrandiMet (Metformin and repaglinide), all of which have different clinical implications.

Low doses of Metformin have shown multiple pathway effects against cancer. Among its most important potential roles in cancer therapy is Metformin's capacity to improve insulin sensitivity, which results in a reduction in insulin levels and a marked reduction in the quantity and activity of Insulin Growth Factor-1 (IGF-1), which is a critical driver of malignant growth in pancreatic cancer. (Bao, et al., Biochem Biophys Acta., Epub (2010)) Researchers from UCLA have identified cross-talk between insulin/IGF-1 and GPCR signaling systems as a key to pancreatic cancer growth, and since Metformin has been shown to block this cross-talk, they propose Metformin as a promising candidate for pancreatic cancer prevention and treatment. Rozengurt, et al., Clin Cancer Res., 16(9):2505-11 (2010). There is also evidence that Metformin assists in a shift from aerobic glycolysis (the “Warburg Effect”) to glucose oxidation, which results in restoration of normal mitochondrial function that, in turn, triggers a renewed capacity for undergoing apoptosis. Martinez-Outschoorn, et al., Cell Cycle, 9(16):3256-76 (2010).

Metformin directly targets the mitochondria respiratory-chain complex I which disrupts ATP production and ROS formation. Down regulation of ATP increases the ratio of AMP:ATP that activates AMPK. AMPK activation by metformin increases insulin sensitivity, and insulin sensitivity is associated with increased type-I interferon IFN-α/β signaling. Metformin has been shown to slow viral replication in Baltimore Class IV RNA viruses similar to SARS-CoV-2 through increasing insulin sensitivity and promoting IFN-α/β signaling. Since pretreatment of cells with type-I IFN can block RNA virus infection, the present disclosure identifies metformin as having great potential for prophylactic effect against RNA viruses (e.g., SARS-CoV-2) because it increases IFN-α/β signaling.

A recent initial study of protein-protein interactions has attempted to contextualize the mechanism of COVID-19 through viral-human protein interaction maps and propose possible antiviral compounds either preclinical, experimental or FDA-approved for further testing. This study has shown that proteins (LARP1, FKBP7) which are regulated by the mTORC1 pathway interact with SARS-CoV-2 N and Orf8 proteins which warrants testing an mTORC1 inhibitor such as metformin. Importantly, inhibition of mTOR has been shown to reduce MERS infection by ˜60% in vitro. The SARS-CoV N protein is involved in packaging the SARS-CoV genome, but it also potently inhibits IRF3 and NFkB, which results in inhibition of IFN-α/β synthesis. Since the SARS-CoV-2 N protein facilitates for viral infection by inhibiting IFN, metformin has the potential to protect against SARS-CoV-2 before and after exposure by promoting IFN-α/β signaling.

Metformin is the front-line type 2 diabetes drug that increases sensitivity to insulin to facilitate glucose uptake, and engages in hypoglycemia-promoting mechanisms including inhibition of gluconeogenesis. It has been used in a clinical setting for over 50 years. Investigation of metformin became widespread after it was determined that diabetic patients taking metformin also had lower risk of developing cancer. Metformin has since been associated with processes of angiogenesis, cell proliferation, metabolism, and inflammation Angiogenic factors, such as VEGFR2 and Ang2, are associated with the development of lung damage such as pulmonary edema and ARDS and are predictive of mortality in patients suffering from such conditions. Metformin downregulates these factors and has been shown to alleviate respiratory conditions, e.g., virus infection associated respiratory failure, pulmonary edema, and/or ARDS.

Even more, metformin is a treatment for diabetes, one of the comorbidities that has been associated with higher case fatality rates from COVID-19, along with cardiovascular disease, and hypertension which are all associated with defects in metabolism and increased inflammation. In the case of COVID-19 infection, it may become important to decrease an innate immune response that can result in tissue-damaging inflammation, sepsis, and progression to ARDS or other virus infection associated respiratory failure, while promoting an efficient adaptive immune response.

It has been hypothesized after studying the 2003 SARS-CoV outbreak, that binding of spike proteins to ACE2 on the surface of pneumocytes downregulates the receptor which allows ACE production of angiotensin II. Angiotensin II binding of type 1a angiotensin II receptor leads to pulmonary vascular permeability, suggesting that viral infection and subsequent lung injury may be due to vascular infiltration. In a double-blind, placebo-controlled clinical study, patients with impaired glucose tolerance (IGT) were given metformin and tested for plasma markers of endothelial activation. Levels of soluble VCAM, and ICAM were decreased suggesting that metformin could protect vasculature through inhibition of endothelial cell and transendothelial infiltration.

In a study with patients suffering from Myobacterium tuberculosis infection, metformin was used as an adjuvant therapy. Patients using metformin had a better survival rate compared to standard TB treatment. This improvement was attributed to the anti-inflammatory effect of metformin through activation of AMPK in conjunction with expansion of a directed T cell response to infection.

In embodiments of the present disclosure, a Metformin dosage regimen will be designed by the attending physician to address the particular metabolic pathways implicated in the disease, for the particular patient, bearing in mind, of course, that in selecting the appropriate dosage in any specific case, consideration must be given to the patient's weight, general health, age, and other factors which may influence response to the drug. In general, in a composition for treating a viral infection as described herein, the dosage range will be 50-2000 mg/day, preferably 500-1000 mg/day. Alternatively, doses of 100 mg, 250 mg, 500 mg, 625 mg, 750 mg, 850 mg, or 1000 mg from one to four times a day, or similar dosing regimens, may be administered. In certain particular embodiments, metformin is administered in a dosing regimen that is or includes doses of 1000 mg/day.

In some embodiments, a combination comprises metformin at a dose of about 250-2000 mg/day. In some embodiments, a combination comprises metformin at a dose of at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1000 mg/day, at least 1100 mg/day, at least 1200 mg/day, at least 1300 mg/day, at least 1400 mg/day, at least 1500 mg/day, at least 1600 mg/day, at least 1700 mg/day, or at least 1800 mg/day. In some embodiments, a combination comprises metformin at a dose of at most 2500 mg/day, at most 2000 mg/day, at most 1750 mg/day, at most 1500 mg/day, at most 1250 mg/day, at most 1000 mg/day, at most 750 mg/day, at most 500 mg/day, at most 450 mg/day, at most 400 mg/day, at most 350 mg/day, or at most 300 mg/day.

In some embodiments, a combination comprises metformin at a dose of about 1000 mg/day.

Low-Dose Naltrexone

Naltrexone is an opioid receptor antagonist used to treat addiction and alcoholism. Low-dose (1/10 the typical daily dose of 50-100 mg) has been shown to have anti-inflammatory effects compared to a normal dose. Low-dose naltrexone was first tested in the clinic in 2007 for Crohn's disease although it has been evaluated over the past 30 years in research studies for fibromyalgia, multiple sclerosis, and cancers. Low-dose naltrexone may also have a role in stress response and emotional well-being which is especially important for front-line workers who must continue their strenuous roles in the face of the COVID-19 pandemic. Further, physiological opioids have immunomodulatory properties, so low-dose naltrexone could also have a similar effect in strengthening the immune system, especially for those who are immune compromised or predisposed to dysregulated chronic inflammation. Low-dose naltrexone is a potent suppressor of inflammatory cytokine release by reducing oxidative stress and inflammation. Daily, oral low-dose naltrexone administration was associated with reduced plasma concentrations of interleukin (IL)-1β, IL-1Ra, IL-2, IL-4, IL-5, IL-6, IL-10, IL-12p40, IL-12p70, IL-15, IL-17A, IL-27, transforming growth factor (TGF)-α, TGF-β, tumor necrosis factor (TNF)-α, and granulocyte-colony stimulating factor (G-CSF).

Suppression of inflammatory signaling by low-haldose naltrexone is critical for high risk patients that may become infected with COVID-19 if they develop ARDS, sepsis, or organ failure. During sepsis, cytokine cascades driven by TNF-α and IL-1β lead to leukopenia and organ failure. Attenuating the immune response without complete blockade is important to facilitate recovery. In a study of endotoxin-induced sepsis in rats, Lin et al found that pretreatment with naltrexone comparatively ameliorated symptoms of shock, bradycardia and hypotension, by reducing superoxide formation, oxidative damage and, importantly decreasing TNF-α. Sepsis is also a major risk factor for developing ARDS so low-dose naltrexone can potentially prevent cases from progressing to fatal cytokine release syndrome and ARDS associated with COVID-19.

Naltrexone is the active ingredient found in name brands including Depade, Vivitrol, and ReVia. Naltrexone is most preferably available in pill form in ReVia (formerly called Trexan). Vivitrol is administered intramuscularly once a month.

Approved dosing regimens for naltrexone include:

TABLE 1
Exemplary Approved Dosing Regimens for Naltrexone

| Indication | Route | Dose |
|---|---|---|
| Alcohol or Opiate | Oral | 50 mg once a day |
| Dependence | | Optionally 25 mg initial dose |
| | | Optionally 100 mg doses |
| | | once/week or every other day |
| | | Optionally 150 mg doses |
| | | every third day |
| | Imtramulscular | 380 mg once a month, |
| | (gluteal injection) | alternating buttocks |

Suitable amounts of Naltrexone for use in accordance with many embodiments of the present disclosure will be 0.1-10 mg, preferably 1.0-10 mg, more preferably 1.5-4.5 mg. For the purposes herein, preferred doses are 3.0 mg and 4.5 mg, in some embodiments rapid release. In certain particular embodiments, naltrexone is administered in a dosing regimen that is or includes 3.5 mg/day. Related compounds such as (S)-N-methylnaltrexone and Nalmefene may also be used in place of Naltrexone, at equivalent Naltrexone dose.

In some embodiments, inclusion and/or dosing of Naltrexone may be reduced or excluded, particularly for patients relying on pain medications with which Naltrexone might or will interfere. Naltrexone can prove very desirable and/or effective in boosting immune system responses, but can decrease efficacy of certain pain medications, sometimes with undesirable effect.

In some embodiments, a combination comprises naltrexone at a dose of about 1.75-7 mg/day. In some embodiments, a combination comprises naltrexone at a dose of at least 0.5 mg/day, at least 1 mg/day, at least 1.5 mg/day, at least 2 mg/day, at least 2.5 mg/day, at least 3 mg/day, at least 3.5 mg/day, at least 4 mg/day, at least 4.5 mg/day, at least 5 mg/day, at least 5.5 mg/day, at least 6 mg/day, or at least 6.5 mg/day. In some embodiments, a combination comprises naltrexone at a dose of at most 10 mg/day, at most 9 mg/day, at most 8 mg/day, at most 7 mg/day, at most 6 mg/day, at most 5 mg/day, at most 4.5 mg/day, at most 4 mg/day, at most 3.5 mg/day, at most 3 mg/day, at most 2.5 mg/day, or at most 2 mg/day.

In some embodiments, a combination comprises naltrexone at a dose of about 3.5 mg/day.

Genistein

Genistein is an isoflavone extracted from fermented soy. It is also referred to as Basidiomycetes Polysaccharide, Fermented Genistein, Fermented Isoflavone, GCP, Genistein Polysaccharide, Génistdéine du Polysaccharide Combiné, Isoflavone Combined Polysaccharide, Polysacaridos Combinados de Genisteina, and Soy Isoflavone Polysaccharide.

Genistein plays an important role in reducing the incidence of breast and prostate cancers. It has been shown that genistein inhibits the activation of NF-kappaB and Akt signaling pathways, both of which are known to maintain a homeostatic balance between cell survival and apoptosis. Furthermore, genistein has been found to have antioxidant properties, and shown to be a potent inhibitor of angiogenesis and metastasis. In addition, genistein works to target endogenous copper which leads to pro-oxidant signaling and consequent cell death.

Genistein has also been shown to downregulate the IGF-1/IGF-1R signaling pathway and inhibit cell growth in hormone refractory PC-3 prostate cancer cells. Treatment with Genistein resulted in a significant inhibition of IGF-1-stimulated cell growth. Treatment with Genistein also strongly attenuated IGF-1-induced β-catenin signaling that correlated with increasing the levels of E-cadherin and decreasing cyclin D1 levels in PC-3 cells. In addition, genistein inhibited T-cell factor/lymphoid enhancer factor (TCF/LEF)-dependent transcriptional activity.

Genistein has also been shown to inhibit VEGF-induced endothelial cell activation by decreasing PTK activity and MAPK activation, resulting in anti-angiogenic activity. Exposure to genistein also decreased activation of JNK and p38, not ERK-1/2, induced by VEGF. It also inhibited activity of MMPs.

Genistein is readily bioavailable. The purest form is commercially available in 99% purity from laboratories including LC Labs, Enzo Life Sciences, BioVision. However, not all forms are suitable for human consumption. Less preferable sources are the soy isoflavone supplements that contain genistein at lower concentrations.

In addition to using the ACE2 receptor, Coronaviruses have been shown to enter into cells via endocytotic pathways. Crucially, genistein inhibits caveolar/clatherin-coated endocytosis, and has been shown to inhibit coronavirus cellular internalization in vitro and in animal models. Genistein drastically lowers viral infectivity by preventing coronaviral particles from entering cells via endocytosis. In addition to this, genistein was identified as an inhibitor of RNA-dependent RNA Polymerase (RdRp).

In many embodiments of the present disclosure, genistein is administered in a dosing regimen that is or includes doses of at least 60 mg/day to 1000 mg/day. In some embodiments, genistein is administered in a dosing regimen that is or includes doses of at least 60 mg/day to 600 mg/day. In certain particular embodiments, genistein is administered in a dosing regimen that is or includes doses 500 mg/day.

In some embodiments, a combination comprises genistein at a dose of about 60-1000 mg/day. In some embodiments, a combination comprises genistein at a dose of at least 50 mg/day, at least 60 mg/day, at least 70 mg/day, at least 80 mg/day, at least 90 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, or at least 950 mg/day. In some embodiments, a combination comprises genistein at a dose of at most 1200 mg/day, at most 1100 mg/day, at most 1000 mg/day, at most 950 mg/day, at most 900 mg/day, at most 850 mg/day, at most 800 mg/day, at most 750 mg/day, at most 700 mg/day, at most 650 mg/day, at most 600 mg/day, at most 550 mg/day, at most 500 mg/day, at most 450 mg/day, at most 400 mg/day, at most 350 mg/day, at most 300 mg/day, at most 250 mg/day, at most 200 mg/day, at most 150 mg/day, or at most 100 mg/day.

In some embodiments, a combination comprises genistein at a dose of about 500 mg/day.

Other

In some embodiments, therapy provided by the present disclosure may include one or more additional agents; alternatively or additionally (e.g., as discussed below), in some embodiments, subjects who receive provided therapy may also be receiving other therapy (i.e., so that inventive therapy is administered in combination with the other therapy).

WO 2014/169221A2 (which is incorporated herein by reference in its entirety) describes a therapy established to be effective for treatment of certain proliferative disorders including multiple cancers. Certain embodiments of this therapy include each of alpha lipoic acid, curcumin, melatonin, metformin, low-dose naltrexone, and genistein, and furthermore include metronomic cyclophosphamide. Cyclophosphamide is reported to have immunosuppressive properties, and, in many embodiments, is excluded from therapy provided in accordance with the present disclosure. In certain embodiments, however, a subject may be receiving cyclophosphamide therapy, including metronomic cyclophosphamide therapy. Alternatively or additionally, in some embodiments, provided therapy may include administration of cyclophosphamide, and specifically may include metronomic dosing of cyclophosphamide.

Cyclophosphamide (CTX) is a synthetic nitrogen mustard alkylating agent used to treat cancers and autoimmune disorders. In the liver, it is converted into the active forms, aldophosphamide and phosphoramide mustard, that have chemotherapeutic activity when they bind to DNA and inhibit DNA replication and initiate cell death.

Cyclophosphamide is only available by prescription for a metronomic dosage of 50 mg or less. Trade names include Cytoxan, Neosar, Clafen, Endoavan, Procytox, Revimmune, Carlovan, Cicloval, Cycloblastin, Cyclobastine, CYCLO-cell, Cyclostin, Cyclostine, Cytophophan, Endoxana, Enduxan, Fosfaseron, Genoxal, Ledoxine, Procytox, and Sendoxan. The chemical structure of Cyclophosphamide is also known as cytophosphane, ciclofosfamida, ciclofosfamide, claphene, cp monohydrate, CPM, cyclophspham, Cyclophosphamid monohydrate, cyclophosphamidum, cyclophosphan, cyclophosphanum, mitoxan, syklofosfamid, zytoxan.

Low-dose cyclophosphamide (CTX) is plasma membrane permeable, and therefore is able to diffuse into the viral particle where it can potentially alkylate and crosslink viral RNA, rendering the virus incapable of replication. In addition, low-dose CTX reduces inflammation and inhibits protein expression levels which might further hamper viral replication and infectivity. In many embodiments of the present disclosure, cyclophosphamide is administered according to a metronomic dosing regimen.

In some embodiments, the provided therapy may include administration of cyclophosphamide at a dose of about 25-50 mg/day. In some embodiments, the provided therapy may include cyclophosphamide at a dose of at least 15 mg/day, at least 20 mg/day, at least 25 mg/day, at least 30 mg/day, at least 35 mg/day, at least 40 mg/day, or at least 45 mg/day. In some embodiments, the provided therapy may include cyclophosphamide at a dose of at most 75 mg/day, at most 70 mg/day, at most 65 mg/day, at most 60 mg/day, at most 55 mg/day, at most 50 mg/day, at most 45 mg/day, at most 40 mg/day, at most 35 mg/day, or at most 30 mg/day. In certain particular embodiments, the provided therapy may include cyclophosphamide at a dose 50 mg/day.

### RNA Viruses

All living organisms on the globe employ DNA as their genetic material, apart from RNA viruses, which can exploit RNA as their genetic material. RNA viruses share a few notable features. First, the mutation rate of RNA viruses is much higher than that of DNA viruses. Therefore, variant viruses are more frequently generated in RNA viruses, a property that enables the RNA viruses to cope with challenges such as antibodies. The high mutation rate is the reason for the difficulty in controlling RNA viruses with antiviral drugs and vaccines. Second, all RNA viruses encode a gene for an RNA-dependent RNA polymerase to synthesize their own RNA genome. This is because RNA viruses, aside from a few exceptions (e.g., hepatitis delta virus and virioids) cannot exploit cellular RNA polymerase, which employs a solely DNA template.

Viral Life Cycle

For both RNA and DNA viruses, the virus life cycle can be divided into three stages—entry, genome replication, and exit. Entry involves attachment, in which a virus particle encounters the host cell and attaches to the cell surface, penetration, in which a virus particle reaches the cytoplasm, and uncoating, in which the virus sheds its capsid. Following the uncoating, the naked viral genome is utilized for gene expression and viral genome replication. Finally, when the viral proteins and viral genomes are accumulated, they are assembled to form a progeny virion particle and then released extracellularly. Virion assembly and the release from the cell constitute the exit.

Entry, the first step of virus infection, involves the recognition of viral receptor by a virus particle. The viral entry can be divided into four steps: attachment, penetration, cytoplasmic trafficking, and uncoating. Attachment refers to the first encounter of virus particles with host cells, which involves two kinds of host proteins on the plasma membrane: (1) attachment factors and (2) viral receptors. The attachment factor on the cell surface recruits and holds the virus particles, thereby facilitating the interaction of the viral particle with the entry receptor.

Following attachment of the virus particle on the target cells, the next step is the penetration into the cytoplasm. The mechanism for the penetration differs, whether enveloped or not. For enveloped viruses, one of the following two mechanisms is used: direct fusion and receptor-mediated endocytosis. For non-enveloped naked viruses, receptor-mediated endocytosis is used for penetration. Direct fusion, as its name implies, is a mechanism in which two membranes (i.e., the viral envelope and cell membrane) fuse. In this case, the viral nucleocapsid is directly delivered to the cytoplasm, leaving the viral envelope behind on the plasma membrane. Retrovirus is an example of a virus that penetrates by direct fusion.

Although some viruses, as described above, penetrate into the cytosol directly through the plasma membrane, most viruses depend on endocytic uptakes, a process termed receptor-mediated endocytosis. Following the engagement of viral particles on the receptor, the virus particle-receptor complex triggers the endocytosis by forming a coated pit on the plasma membrane, leading to endosome formation. As a result, the virus particle becomes located inside the endosome. The next step is to breakdown the endosome to penetrate to the cytoplasm. The process of endosome breakdown differs whether enveloped or not. For enveloped viruses, the membrane fusion between the viral envelope and the endosomal membrane triggered by acidic pH at early endosome causes the endosome breakdown. More precisely, the fusion peptide embedded on the envelope glycoprotein becomes exposed (i.e., activated) as a consequence of conformational change upon low pH; then the fusion is triggered by the fusion peptide. For non-enveloped naked viruses, the endosome lysis is induced by one of the capsid proteins. In other words, membrane fusion is the mechanism of penetration for envelope viruses, while membrane lysis is the mechanism of penetration for non-enveloped viruses.

Receptor-mediated endocytosis typically proceeds via a clathrin-dependent manner. Receptor-mediated endocytosis is the mechanism intrinsic to the cells, which is utilized to take extracellular molecules into the cells. Clathrin-mediated endocytosis, which is also the pathway utilized for uptake of LDL, is employed by many viruses, such as influenza virus and adenovirus. Upon the binding of the virus particle with the receptor, a clathrin-coated pit is formed, as clathrins are recruited near the plasma membrane. Following the formation of an endocytic vesicle, the vesicles are fused with early endosomes. The virus particles are now located inside the early endosomes.

As the virus particles approach to the site of replication, from the cell periphery to the perinuclear space, the viral genome becomes exposed to cellular machinery for viral gene expression, a process termed uncoating. Uncoating is often linked with the endocytic route or cytoplasmic trafficking. For viruses that replicate in the nucleus, the viral genome needs to enter the nucleus via a nuclear pore. Multiple distinct strategies are utilized, largely depending on their genome size. For the virus with a smaller genome, such as polyomavirus, the viral capsid itself enters the nucleus. For viruses with a larger genome, the docking of nucleocapsids to a nuclear pore complex causes a partial disruption of the capsid (e.g., adenovirus) or induces a minimal change in the viral capsid (e.g., herpes virus), allowing the transit of DNA genome into the nucleus.

Exit can be divided into three steps: capsid assembly, release, and maturation. The capsid assembly follows as the viral genome as well as the viral proteins abundantly accumulates. For naked viruses, the virus particles are released via cell lysis of the infected cells. Thus, no specific exit mechanism is necessary, because the cell membrane that traps the assembled virus particles are dismantled. Examples of naked viruses are polyomavirus (i.e., SV40) and adenovirus. By contrast, in cases of enveloped viruses, envelopment, a process in which the capsids become surrounded by lipid bilayer, takes place prior to the release. The last step of the virus particle assembly is “maturation,” a process that occurs extracellularly following release. For picornavirus and retrovirus, maturation is an essential step to acquire infectivity. In case of retrovirus, the cleavage of the Gag polyprotein by the viral PR protein (aspartate protease) occurring in the released virion is accompanied with a considerable morphological transition such as the condensation of the capsid structure. Importantly, such a maturation process confers the particle its infectivity.

Single-Stranded RNA Viruses (Positive-Sense, Negative-Sense RNA Viruses)

RNA viruses can be further classified according to the sense or polarity of their RNA into negative-sense and positive-sense. Positive-sense viral RNA is similar to mRNA and thus can be immediately translated by the host cell. Negative-sense viral RNA is complementary to mRNA and thus must be converted to positive-sense RNA by an RNA-dependent RNA polymerase before translation. As such, purified RNA of a positive-sense virus can directly cause infection though it may be less infectious than the whole virus particle. Purified RNA of a negative-sense virus is not infectious by itself as it needs to be transcribed into positive-sense RNA; each virion can be transcribed to several positive-sense RNAs.

Eight virus families whose members infect vertebrates are currently known to possess single-stranded, positive-sense RNA genomes: the families Picornaviridae, Caliciviridae and Hepeviridae have non-enveloped capsids, whereas the families Flaviviridae, Togaviridae, Arteriviridae and Coronaviridae (e.g., SARS-CoV and SARS-CoV-2) are characterized by enveloped capsids. They all have in common the property of using their own genome as messenger RNA (mRNA), from which they synthesize one or several polyproteins that are subsequently cleaved into individual proteins by viral or cellular proteases. These viruses possess the genetic information for the synthesis of an RNA-dependent RNA polymerase. This enzyme transcribes the positive RNA strand as well as the complementary negative RNA strands, which arise as intermediate products of genome replication. In the course of this process, the new genomic RNA molecules are generated from the second transcription step. The classification into the different taxonomic families depends on the number, size, position and orientation of viral genes in the RNA molecule, the number of different polyproteins that are synthesized during viral infection and the existence of an envelope as a virion component. The (+)ssRNA viruses are classified into three orders—the Nidovirales, Picornavirales, and Tymovirales—and 33 families, of which 20 are not assigned to an order. A broad range of hosts can be infected by (+)ssRNA viruses, including bacteria (the Leviviridae), eukaryotic microorganisms, plants, invertebrates, and vertebrates.[17]

Negative sense ssRNA viruses need RNA polymerase to form a positive sense RNA. The positive-sense RNA acts as a viral mRNA, which is translated into proteins for the production of new virion materials. With the newly formed virions, more negative sense RNA molecules are produced. The genome size of a negative RNA virus is between 10 kb to 30 kb. Two genome subgroups can be distinguished, nonsegmented and segmented. In viruses with nonsegmented genomes, the first step of replication is transcription of the negative strand by RdRp to form various monocistronic mRNA that code for individual viral proteins. A positive strand copy is formed to serve as template for the production of the negative genome. This replication takes place in the cytoplasm. In viruses with segmented genomes, replication occurs in the nucleus and the RdRp produces one monocistronic mRNA strand from each genome segment. The principal difference between the two types is the location of replication. One phylum, two subphyla, six classes, eight orders and twenty one families are currently recognised in this group. A number of unassigned species and genera are yet to be classified. Viruses of the families Arenaviridae, Orthomyxoviridae, Paramyxoviridae, and Pneumoviridae are able to infect vertebrates. Viruses of the families Bunyaviridae and Rhabdoviridae are able to infect vertebrates, arthropods, and plants. Viruses of the genus Tenuivirus only infect plants. A few viruses known to infect humans include Marburg virus, Ebola, measles, mumps, rabies, and influenza.

Double-Stranded RNA Viruses

The double-stranded RNA viruses represent a diverse group of viruses that vary widely in host range (humans, animals, plants, fungi, and bacteria), genome segment number (one to twelve), and virion organization (Triangulation number, capsid layers, spikes, turrets, etc.). Members of this group include the rotaviruses, which are the most common cause of gastroenteritis in young children, and picobirnaviruses, which are the most common virus in fecal samples of both humans and animals with or without signs of diarrhea. Bluetongue virus is an economically important pathogen of cattle and sheep. In recent years, progress has been made in determining, at atomic and subnanometeric levels, the structures of a number of key viral proteins and of the virion capsids of several dsRNA viruses, highlighting the significant parallels in the structure and replicative processes of many of these viruses.

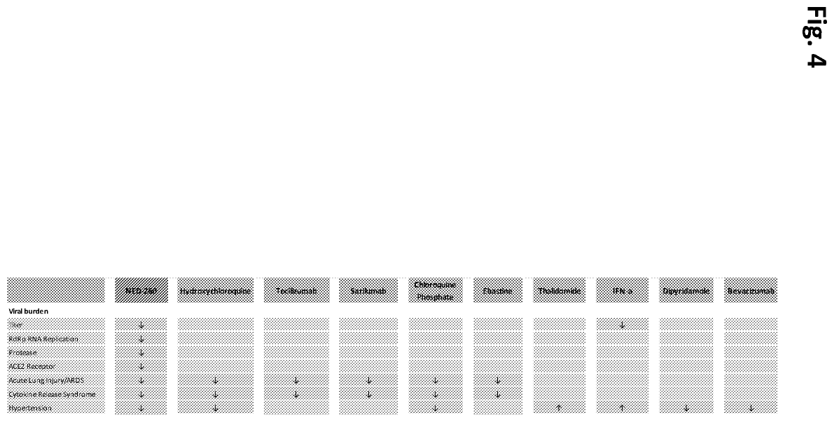

Double stranded RNA viruses are all nonenveloped and possess icosahedral capsids. They have segmented genomes, and two families of dsRNA viruses infect humans. Viruses in the Reoviridae family include rotavirus, so named because the virion looks like a wheel (rota means “wheel” in Latin; FIG. 4.9). Rotavirus has 11 genome segments and is the major cause of childhood diarrhea. Picobirnaviruses are another family of dsRNA viruses that infect humans, but they are bisegmented, only having two genome segments that together are around 4.2 kb in length (the name of the viral family means “small two-RNA viruses,” referring to the two dsRNA genome segments). Human picobirnaviruses have been isolated from diarrhea, although the association of the virus as a cause of a specific disease is currently unclear. Double-stranded RNA virus families include Amalgaviridae, Birnaviridae, Chrysoviridae, Cystoviridae, Endornaviridae, Hypoviridae, Megabirnaviridae, Partitiviridae, Picobirnaviridae, Quadriviridae, Reoviridae, and Totiviridae.

### DNA Viruses

In some embodiments, the present disclosure can be adapted for treating and preventing DNA virus infections. A DNA virus is a virus that has DNA as its genetic material and replicates using a DNA-dependent DNA polymerase. The nucleic acid is usually double-stranded DNA (dsDNA) but may also be single-stranded DNA (ssDNA). DNA viruses belong to either Group I or Group II of the Baltimore classification system for viruses. Single-stranded DNA is usually expanded to double-stranded in infected cells. Although Group VII viruses such as hepatitis B contain a DNA genome, they are not considered DNA viruses according to the Baltimore classification, but rather reverse transcribing viruses because they replicate through an RNA intermediate. Notable diseases like smallpox, herpes, and the chickenpox are caused by such DNA viruses.

Group I

Group I DNA viruses viral families include at least Myoviridae, Podoviridae, Siphoviridae, Alloherpesviridae, Herepesviridae, Malacoherpesviridae, Lipothrixviridae, Rudiviridae, Adenoviridae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae, Lavidaviridae, Marseilleviridae, Mimiviridae, Nimaviridae, Nudiviridae, Pandoraviridae, Papillomaviridae, Phycodnaviridae, Plasmaviridae, Polydnaviruses, Polyomaviridae, Poxviridae, Sphaerolipoviridae, Tectiviridae, Tristromaviridae, and Turriviridae.

Genome organization within this group varies considerably. Some have circular genomes (Baculoviridae, Papovaviridae and Polydnaviridae) while others have linear genomes (Adenoviridae, Herpesviridae and some phages). Some families have circularly permuted linear genomes (phage T4 and some Iridoviridae). Others have linear genomes with covalently closed ends (Poxviridae and Phycodnaviridae).

Group II

Group II DNA virus families include at least Anelloviridae, Bacilladnaviridae, Bidnaviridae, Circoviridae, Geminiviridae, Genomoviridae, Inoviridae, Microviridae, Nanoviridae, Parvoviridae, Smacoviridae, and Spiraviridae

Although bacteriophages were first described in 1927, it was only in 1959 that Sinshemer working with phage Phi X 174 showed that they could possess single-stranded DNA genomes. Despite this discovery, until relatively recently it was believed that most DNA viruses contained double-stranded DNA. Recent work, however, has shown that single-stranded DNA viruses can be highly abundant in seawater, freshwater, sediments, terrestrial and extreme environments, as well as metazoan-associated and marine microbial mats. Many of these “environmental” viruses belong to the family Microviridae. However, the vast majority has yet to be classified and assigned to genera and higher taxa. Because most of these viruses do not appear to be related, or are only distantly related to known viruses, additional taxa will have to be created to accommodate them.

### Exemplary Viruses Treated by Provided Therapy

SARS-CoV-2 Virology

SARS-CoV-2 is a novel virus originating from Wuhan, China in late 2019 that belongs to the Class IV of the Baltimore classification. In other words, SARS-CoV-2 is a positive-sense single stranded RNA (+ssRNA) virus that can be directly accessed by host ribosomes in the cytosol to create viral proteins. SARS-CoV-2 is a new virus belonging to the Coronavirus family, which includes less pathogenic strains responsible for the common cold, as well as the viruses responsible for SARS and MERS. It is genetically related to the coronavirus responsible for the SARS outbreak in 2003; the closest identified relative was isolated from bats.

SARS-CoV-2 is spread primarily via droplet, though it can be aerosolized and can persist on plastic and stainless steel surfaces for up to 72 hours. SARS-CoV-2 contains its +ssRNA genome within a spherical particle that has an exterior comprised of phospholipids derived from the host cell membrane. The membrane has glycoprotein ‘spikes’ pointing outwards from the particle. These membrane ‘spikes’ are also referred to as the S protein, and the S protein forms a trimer that binds to ACE2 receptors on type 2 pneumocytes.

This virus utilizes a multitude of complex mechanisms to express genes encoded in the +ssRNA including an internal ribosomal entry site (IRES), frameshifting, multiple types of proteolytic processing of polyproteins, and subgenomic messenger RNAs (mRNAs). The virus potentially encodes 24 different proteins that perform functions related to RNA replication, repression of host cell translation, RNA packaging, and infectious viral particle formation. SARS-CoV-2 is never in the form of a DNA intermediate because this virus encodes its own RdRp and helicase. The RdRp will create a negative-sense ssRNA template from the +ssRNA SARS-CoV-2 genome. Afterwards, the RdRp is able to utilize this template to produce many copies of the +ssRNA SARS-CoV-2 genome. The +ssRNA genome copies are packaged into particles bearing the S protein that bud off of the host cell membrane surface to continue the next cycle of infection, and this is commonly referred to as ‘virus shedding.’

The viral RNA encodes proteins that are necessary for its replication and infectivity including, its own RdRp, host translational repressor, papain-like protease, 3C-like protease, endoribonuclease, helicase, and 5′ capping enzyme. Only some of these proteins are potentially druggable. For example, proteases are particularly interesting therapeutic targets because of the successes of other viral protease inhibitors. Curcumin is an inhibitor of the 3C-like protease which is needed to process and activate viral proteins. Genistein is an inhibitor of the RdRP which is needed to create a template of the genome that will enable the production of many copies of the viral genome.

SARS-CoV-2 Clinical Manifestations

Severe COVID-19 is characterized by the cytokine storm and increased neutrophil migration to the lungs. Protection against reinfection is unclear; CD4+ Th1 and CD8+ cells both are part of the immune response to the virus. Lymphopenia is seen in COVID-19, presumably due to bone marrow suppression by the antiviral response.

Common symptoms include cough, fever, and fatigue; however, sputum, shortness of breath, myalgias, sore throat, headache, nasal congestion, anorexia, loss of taste, and nausea/vomiting/diarrhea have also been reported. The elderly and those with comorbid conditions (most clearly cardiovascular disease, respiratory conditions, diabetes and cancer) are at higher risk for a more severe disease course and death. In addition, smokers are more likely to have serious complications. RT-PCR on respiratory samples is the current gold standard for diagnosis. Serologic antibody tests are undergoing FDA approval. Common lab findings include: ↑lymphocytes, ↑platelets, and ↓CRP. Higher inflammatory markers are seen in more severe disease. Common chest CT findings include: bilateral ground glass opacities, consolidations, and “crazy paving” patterns.

Clinical outcomes include mild disease, pneumonia, severe pneumonia, ARDS, and septic shock. Case fatality rate is estimated to be ˜2%, but many cases likely remain undiagnosed in the United States at this time (04/01/2020). Patients exhibiting mild symptoms are to undergo ˜14-day home quarantine. Patients are only admitted if there is significant risk of decompensation. Patients exhibiting moderate to severe symptoms are admitted to the hospital in an airborne isolation room. ICU level care is necessary for advanced ventilatory support or support for 2+ organ systems. Standard supportive measures include: isolation and conservative fluid management. Possible supportive measures in the case of comorbid conditions include: empiric antibiotics, antiviral drugs that mitigate symptoms, bronchodilators, and ventilatory support.

In regard to investigational therapies and vaccine development, there are currently no FDA-approved treatments directed against COVID-19 at this time (04/01/2020). A variety of therapies are under investigation, however. These include repurposing of antivirals (remdesivir, lopinavir/ritonavir), antimalarials (chloroquine/hydroxychloroquine), and immunosuppressive medications (tocilizumab), or antibodies against SARS-CoV-2 analogs/SARS-CoV. For example, Regeneron, Sanofi, and Roche plan to run trials focused on antibodies against the IL-6 receptor commonly used to treat rheumatoid arthritis. It is expected that COVID-19 vaccine development will take a minimum of one year.

It is worth noting that convalescent antibody therapy is currently being considered as a prophylactic strategy for healthcare workers and first responders. The feasibility of such an approach being implemented on a massive scale that would provide protection to the vast number of front-line healthcare workers and first responders is low because of challenges related to antibody procurement, costs, quality control, cold chains of supply, and delivery to front lines. In addition, this approach would probably not protect against a coronavirus that causes the next pandemic of the future.

MERS-CoV Virology

In 2012, a new human disease called Middle East respiratory syndrome (MERS), having a high mortality rate, emerged in the Middle East. MERS-CoV is comparable to severe acute respiratory syndrome coronavirus (SARS-CoV), which killed almost 10% of the affected individuals in China between 2002 and 2003. The first MERS patient reported in Saudi Arabia in June of 2012 were possibly infected by direct or indirect transmission of the virus from dromedary camels. Moreover, MERS-CoV similar to the isolates from dromedary camels and humans was found in bats. Evidence suggests that MERS-CoV can be transmitted to humans via both animals and humans. However, the successive epidemics of MERS indicate that the pathogen has spread to various parts of the world predominantly via interhuman transmission.

The MERS-CoV genome is 30,119 nucleotides long and contains 11 open reading frames (ORFs). The single positive-stranded RNA genome has 5′- and 3′-untranslated regions that are 278 and 300 nucleotides in length, respectively. The 5′ end comprises two overlapping ORFs, ORF1a and ORF1b, which are translated to yield two large polyproteins, polyprotein 1a (pp1a) and polyprotein lab (pp lab). These polyproteins are cleaved into 16 functional nonstructural proteins (nsps) by the proteolytic activity of two viral proteases called papain-like protease (PLpro) and 3C-like protease (3CLpro) after their self-cleavage from pp1ab. Proteolytic processing of MERS-CoV polyproteins is required for the activation of viral replication. In addition to these two proteases, the two ORFs encode other nsps that are responsible for viral RNA-dependent RNA polymerase activity (nsp12), RNA helicase activity (nsp13), exoribonuclease activity (nsp14), endoribonuclease activity (nsp15) and methyltransferase activity (nsp16). The role of nsp14 is essential, as it is involved in proofreading by monitoring the mutation rate, a unique feature for an RNA virus. More genes downstream of ORF1ab encode structural and accessory proteins. Spike (S), envelope (E), membrane (M) and nucleocapsid (N) proteins are all structural proteins, whereas the accessory proteins, unique to this lineage of viruses, are encoded by ORF3, ORF4a, ORF4b, ORF5 and ORF8b. Although the exact function of these accessory proteins is still unknown, some recent studies have shown that they may have an important role in evading the host immune response.

MERS-CoV enters the host through its S protein, a type I transmembrane glycoprotein with 1353 amino acids (aa) that exists on the virion surface as a trimer. Subsequently, it is recognized by cluster of differentiation 26 (CD26) (also known as dipeptidyl peptidase 4 (DPP4)), which facilitates the infection of the host cells. SARS-CoV uses angiotensin-converting enzyme 2 as a functional receptor. MERS-CoV and SARS-CoV differ in their cellular selection for infection, possibly owing to their selective binding with different receptors.

MERS-CoV infection was initially thought to spread by zoonotic events via bats as phylogenetic studies revealed that it is genetically connected to Tylonycteris bat coronavirus HKU4 (BatCoV-HKU4) and Pipistrellus bat coronavirus HKU5 (BatCoV-HKU5). However, evidence indicates that MERS-CoV originated from dromedary camels. A serological study suggests that almost 90% of all camels in Africa and the Middle East were seropositive for MERS-CoV, whereas other animals such as sheep, goats and cows were found to be negative. A population-based seroepidemiologic study suggests that the seroprevalence of the virus was several folds higher in people who were exposed to camels compared with that in the general population. Moreover, antibodies against MERS-CoV were found in samples obtained from camels in Saudi Arabia in 1993, which reinforces the hypothesis that dromedary camels are most likely the main reservoirs of MERS-CoV. In contrast, no seroreactivities were reported in the blood samples obtained from blood donors and abattoir workers in Saudi Arabia during 2012. MERS-CoV was detected in camels in Egypt that were locally raised or imported from countries where no MERS cases were reported. The mode of transmission is still unknown but is suspected to be through saliva during direct contact with infected camels or through consumption of milk or uncooked meat.

Secondary infection may occur through droplets or contact, and the virus could spread either via air or fomites. A few recent studies on infected patients showed that the most common MERS-CoV infection causes acute pneumonia and renal failure and that almost every patient developed respiratory problems. In addition, at least one-third of the studied patients were also reported to have abdominal disorders. Other effects include inflammation of the pericardium, consumptive coagulopathy, increase in leukocytes and neutrophils, and low levels of lymphocytes, platelets and red blood cells. Moreover, hyponatremia and low blood levels of albumin were detected during the case study.

MERS-CoV Clinical Manifestations

A wide clinical spectrum of MERS-CoV infection has been reported ranging from asymptomatic infection to acute upper respiratory illness, and rapidly progressive pneumonitis, respiratory failure, septic shock and multi-organ failure resulting in death. Most MERS-CoV cases have been reported in adults (median age approximately 50 years, male predominance), although children and adults of all ages have been infected (range 0 to 109 years). Most hospitalized MERS-CoV patients have had chronic co-morbidities. Among confirmed MERS-CoV cases reported to date, the case fatality proportion is approximately 35%.

Limited clinical data for MERS-CoV patients are available; most published clinical information to date is from critically ill patients. At hospital admission, common signs and symptoms include fever, chills/rigors, headache, non-productive cough, dyspnea, and myalgia. Other symptoms can include sore throat, coryza, nausea and vomiting, dizziness, sputum production, diarrhea, and abdominal pain. Atypical presentations including mild respiratory illness without fever and diarrheal illness preceding development of pneumonia have been reported. Patients who progress to requiring admission to an intensive care unit (ICU) often have a history of a febrile upper respiratory tract illness with rapid progression to pneumonia within a week of illness onset.

The median incubation period for secondary cases associated with limited human-to-human transmission is approximately 5 days (range 2-14 days). In MERS-CoV patients, the median time from illness onset to hospitalization is approximately 4 days. In critically ill patients, the median time from onset to intensive care unit (ICU) admission is approximately 5 days, and median time from onset to death is approximately 12 days. In one series of 12 ICU patients, the median duration of mechanical ventilation was 16 days, and median ICU length of stay was 30 days, with 58% mortality at 90 days. Radiographic findings may include unilateral or bilateral patchy densities or opacities, interstitial infiltrates, consolidation, and pleural effusions. Rapid progression to acute respiratory failure, acute respiratory distress syndrome (ARDS), refractory hypoxemia, and extrapulmonary complications (acute kidney injury requiring renal replacement therapy, hypotension requiring vasopressors, hepatic inflammation, septic shock) has been reported.

Laboratory findings at admission may include leukopenia, lymphopenia, thrombocytopenia, and elevated lactate dehydrogenase levels. Co-infection with other respiratory viruses and a few cases of co-infection with community-acquired bacteria at admission has been reported; nosocomial bacterial and fungal infections have been reported in mechanically-ventilated patients. MERS-CoV virus can be detected with higher viral load and longer duration in the lower respiratory tract compared to the upper respiratory tract, and has been detected in feces, serum, and urine.

Duration of MERS-CoV shedding in the respiratory tract is typically longer in more severely ill patients than mildly ill patients, and evidence of virus has been detected in survivors for a month or more after onset. Limited data are available on the duration of extrapulmonary MERS-CoV shedding.

No specific treatment for MERS-CoV infection is currently available. Clinical management includes supportive management of complications and implementation of recommended infection prevention and control measures.

SARS-CoV Virology

Severe acute respiratory syndrome (SARS) is caused by a novel coronavirus, now called the SARS coronavirus (SARS-CoV). Over 95% of well characterized cohorts of SARS have evidence of recent SARS-CoV infection and experimental infection of cynomolgous macaques with SARS-CoV leads to a SARS-like disease associated with lung pathology and giant cells reminiscent of the human disease. Investigations during some outbreaks of SARS have also revealed coinfection with human metapneumovirus or other pathogens in a proportion of patients. Whether such coinfections contribute to enhancing the pathogenesis or transmission of the disease is still uncertain.

The primary human receptor of the virus is angiotensin-converting enzyme 2 (ACE2), first identified in 2003.

The genome of SARS-CoV indicates that it is a novel virus within the family coronaviridae, not closely related to any of the human or animal coronaviruses known to date. Healthy humans have no serological evidence of past SARS-CoV infection. Closely related viruses have recently been isolated from animals such as civet cats. It is probable that SARS-CoV was an animal virus that adapted to human-human transmission in the recent past. The presence of this animal reservoir implies that it is possible for this virus to again cross into humans and initiate outbreaks of disease in the future. Many respiratory viruses, including coronaviruses have a seasonality and that of SARS-CoV is yet unknown. It is therefore possible that, similar to other coronaviruses, winter may be more conducive to the transmission of SARS-CoV.

SARS-CoV can be detected by culture in vero-E6 or FRhK-4 cells or by reverse transcriptase polymerase chain reaction (RT-PCR) in respiratory secretions, faeces and urine, and also less frequently in blood. The infection is thus not restricted to the respiratory tract alone and the presence of diarrhea in a proportion of patients is one indication of this fact. In contrast to most other respiratory viruses, SARS-CoV viral load in the respiratory tract and faeces is low in the first few days of the illness but peaks around day 11 of the disease. This accounts for the low sensitivity of the first generation diagnostic tests in the first few days of illness. It may also explain in part the unusual predilection of this virus to spread among health care workers, as patients are usually most infectious later in the illness by which time many of them are already hospitalized.

Respiratory specimens and feces are useful clinical specimens for RT-PCR diagnosis, but feces is a less satisfactory specimen in the first 5 days of illness. Nasopharyngeal aspirates are respiratory specimens of choice, but throat swabs can also be used, though with lower diagnostic yield. When patients are producing sputum, it is also an excellent clinical specimen, however, in most patients the cough is non-productive. Second generation RT-PCR assays are able to detect SARS-CoV in nasopharyngeal aspirates of approximately 80% of patients with SARS within the first 3 days of illness. Seroconversion for SARS-CoV using immunofluorescence on infected cells is an excellent method of confirming the diagnosis, although antibody responses appear only around day 10 of the illness and such diagnosis is retrospective in nature. Since some patients have a delayed antibody response it is advisable to obtain a convalescent serum at least 21 days, but ideally 28 days, after the onset of the disease, especially if patients have been on high dose steroid therapy in the acute stage of the illness.

Understanding the pathogenesis of SARS is bedeviled by the fact that most available autopsy tissues are from patients dying later in the illness, at a time when the initial viral pathology is obscured by secondary infections or changes due to ventilator therapy, steroids and other immune modulators. Despite these limitations on autopsy material, it has been possible to determine that there are two phases of SARS pneumonia. Within the first 10 days the histological picture is that of acute phase diffuse alveolar damage (DAD) with a mixture of inflammatory infiltrate, oedema and hyaline membrane formation. Desquamation of pneumocytes is prominent and consistent. One report initially published in Chinese but later in English in another journal mentioned viral intracytoplasmic inclusion bodies but these were not identified in the Hong Kong or Singapore cases. After 10 days of illness the picture changes to one of organizing DAD with increased fibrosis, squamous metaplasia and multinucleated giant cells. The giant cells that have also been seen in the experimentally induced SARS pneumonia in primates and coronaviruses are known to give rise to syncitia. Histologically, the appearance is not distinctive enough to make a diagnosis without viral detection. In humans, as well as in primates, SARS-CoV was identified in the lung.

SARS-CoV Clinical Manifestations

The incubation period of SARS is generally between 2 and 10 days although it has been estimated to be 6.4 days (95% CI 5.2-7.7) with the mean time from onset of clinical symptoms to hospital admission between 3 and 5 days. The major clinical features include persistent fever, chills/rigor, myalgia, malaise, dry cough, headache and dyspnea. Less common symptoms include sputum production, sore throat, coryza, dizziness, nausea, vomiting and diarrhea. Watery diarrhea has been reported in 73% of a group of patients one week down the clinical course in a community outbreak linked to a faulty sewage system, presumably due to involvement of the gastrointestinal tract via the fecal oral route. Nevertheless, these clinical symptoms are rather non-specific and may mimic influenza or atypical pneumonia of other causes such as mycoplasma, chlamydia and legionella. Older subjects may present with decrease in general well-being, poor feeding, fall/fracture, and in some cases, delirium, without the typical febrile response. Physical examination of patients with SARS may reveal fever, tachycardia, tachypnea and inspiratory crackles at the lung bases in some cases.

Lymphopenia, features of low grade disseminated intravascular coagulation (thrombocytopenia, prolonged activated partial thromboplastin time, elevated D-Dimer), elevated alanine transminases (ALT), lactate dehydrogenase (LDH) and creatinine kinase (CPK) are commonly observed in SARS patients with active disease. These laboratory features, together with the clinical features, may help in the clinical diagnosis of the disease. Lactate dehydrogenase, ALT and CPK tend to improve along with clinical and radiological improvement. The CD4 and CD8 lymphocyte counts fall early in the course of SARS, whereas low counts of CD4 and CD8 at presentation are associated with adverse outcome.

The clinical course of SARS appears to follow a typical pattern in many cases. Phase 1 (viral replication) is associated with increasing viral load and clinically characterized by fever, myalgia and other systemic symptoms that generally improve after a few days. Phase 2 (immunopathological damage) is characterized by recurrence of fever, oxygen desaturation, radiological progression of pneumonia with falls in viral load. The majority of patients will improve with a combination of ribavirin and intravenous pulse steroid therapy but 20-36% may require ICU admission, and 13-26% may progress into acute respiratory distress syndrome (ARDS) necessitating invasive ventilatory support. Compared with adults and teenagers, SARS seems to run a less aggressive clinical course in younger children and children aged below 13 years typically do not require supplemental oxygen.

The average hospital length of stay for the majority of patients during the recent epidemic in Hong Kong was 21 days with a 21 day mortality between 3.6 and 10%. Some of the patients received physiotherapy as outpatients while about 15% of patients required a period of rehabilitation as inpatients. The prognostic factors associated with a poor outcome (ICU admission or death) include advanced age, chronic hepatitis B treated with lamivudine, high initial LDH, high peak LDH, high neutrophil count on presentation, diabetes mellitus or other comorbid conditions and low CD4 and CD8 lymphocyte counts at presentation.

### Psychosocial Factors And Comorbidities

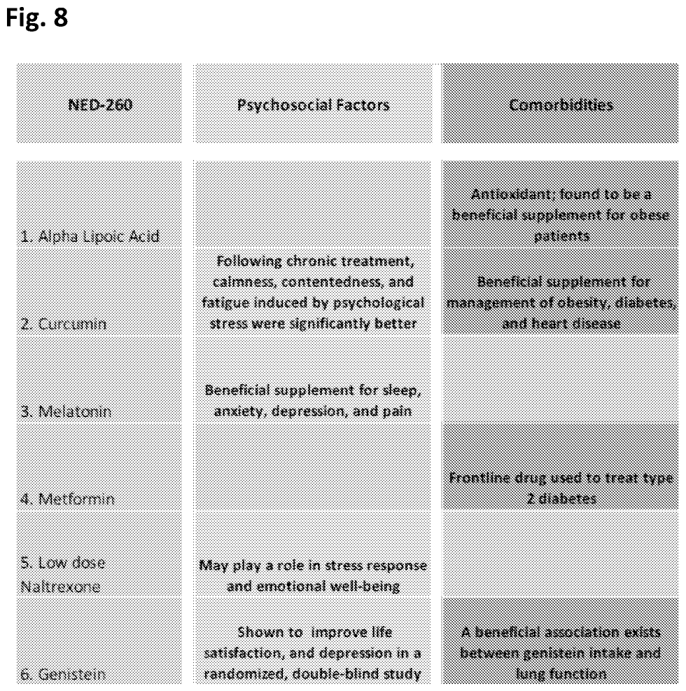

The present disclosure recognizes the mental health toll that is experienced by healthcare workers working on the front-lines during a pandemic. Indeed, the intense stress that healthcare workers face on a daily basis during a pandemic can trigger exhaustion, insomnia, anxiety, depression, hypertension, cardiovascular disease and suicide. In many embodiments of the present disclosure, combinations of agents are provided which have a role in stress response and emotional well-being which is especially important for front-line workers who must continue their strenuous roles during a pandemic (FIG. 8).

The present disclosure further appreciates that the presence of underlying conditions is related to cardiovascular risk in patients suffering from viral infection (e.g., COVID-19), and this could exacerbate the recently reported issues surrounding clotting/stroke. According to the CDC Morbidity and Mortality Weekly Report, approximately 90% of hospitalized patients have comorbidities with hypertension, diabetes, cardiovascular disease, obesity and chronic lung disease contributing the most to mortality (04/17/2020). The present disclosure provides combinations of agents that have demonstrated efficacy as therapeutics or supplements for long-term management of these comorbidities (FIG. 8). Indeed, the present disclosure bridges the divide between bona fide therapeutics and nutritional supplements in order to comprehensively treat viral infection (e.g., COVID-19) and comorbidities while also providing holistic support for mental health.

In some embodiments of the present disclosure, a combination to simultaneously treat or prevent viral infection, comorbidities, and mental health is or comprises three or more of alpha lipoic acid, curcumin, genistein, melatonin, metformin, and naltrexone. In some embodiments, a combination to simultaneously treat or prevent viral infection, comorbidities, and mental health is modified to include or omit certain agents based on the mental health and/or comorbidities of a subject.

### Administration of Provided Therapy

In general, agents included in inventive combination therapies may be administered in any form, preferably as a tablet, powder, or liquid, formulated into a pharmaceutically acceptable carrier or excipient, depending on the condition of the patient. In some embodiments, providing custom tailored dosages eliminates the need for pre-formulated capsules and tablets. Additionally, non-active ingredients well known in the art, such as binders, fillers, coatings, preservatives, coloring agents, flavoring agents and other additives may optionally be formulated with one or more administered agents, or left out completely if there is a risk of negative side effects to the patient such as increased the risk of intestinal inflammation or interference with the absorption of particular compounds.

In some embodiments, one or more agents included in an inventive therapeutic regimen is administered according to a metronomic regimen.

In some embodiments, inventive therapeutic regimens are added to a different antiviral regimen. In some such embodiments, the compositions described herein supplement the action of a different antiviral regimen by addressing additional processes and pathways not addressed by said different antiviral regimen. In many embodiments, effectiveness of any suitable antiviral used for the treatment of an RNA virus infection, more particularly for the treatment of coronavirus related viral infections, will be improved by adding compositions and combination therapies according to the present disclosure.

### Other Antiviral Treatments

In some embodiments, the present disclosure can be used with one or more additional antiviral therapies. In some embodiments, the present disclosure is coadministered with one or more additional antiviral therapies. In some embodiments the present disclosure is administered in overlapping regimens with one or more antiviral therapies. In some embodiments, the present disclosure is sequentially administered with one or more antiviral therapies. In certain embodiments, the at least one of the one or more additional antiviral therapies is a repurposed antiviral therapy, where the antiviral therapy is not indicated for the virus infection to be treated or prevented. In some embodiments, the one or more additional antiviral therapies comprise one or more of the antiviral therapies and proposed antiviral therapies listed below.

### Abacavir Sulfate (ZIAGEN™)

ZIAGEN is the brand name for abacavir sulfate, a synthetic carbocyclic nucleoside analogue with inhibitory activity against HIV. The chemical name of abacavir sulfate is (1S,cis)-4-[2-amino-6-(cyclopropylamino)-9H-purin-9-yl]-2-cyclopentene-1-methanol sulfate (salt) (2:1). Abacavir sulfate is the enantiomer with 1S, 4R absolute configuration on the cyclopentene ring. It has a molecular formula of (C14H18N6O)2 31·H2SO4 and a molecular weight of 670.76 daltons.

Dosage and Administration

ZIAGEN Tablets: In some embodiments, a tablet contains abacavir sulfate equivalent to 300 mg abacavir.

ZIAGEN Oral Solution: In some embodiments, each mL of the solution contains abacavir sulfate equivalent to 20 mg of abacavir.

ZIAGEN may be taken with or without food. Adults: In some embodiments, a recommended oral dose of ZIAGEN for adults is 300 mg twice daily in combination 378 with other antiretroviral agents. Adolescents and Pediatric Patients: In some embodiments, a recommended oral dose of ZIAGEN for adolescents and pediatric patients 3 months to up to 16 years of age is 8 mg/kg twice daily (up to a maximum of 300 mg twice daily) in combination with other antiretroviral agents.

In some embodiments, abacavir sulfate is administered at a dose of 20-300 mg/day.

Acyclovir (ZOVIRAX®)

ZOVIRAX is the brand name for acyclovir, a synthetic nucleoside analogue active against herpesviruses. Acyclovir sodium for injection is a sterile lyophilized powder for intravenous administration only. Each 500-mg vial contains 500 mg of acyclovir and 49 mg of sodium, and each 1,000-mg vial contains 1,000 mg acyclovir and 98 mg of sodium. Reconstitution of the 500-mg or 1,000-mg vials with 10 mL or 20 mL, respectively, of Sterile Water for Injection, USP results in a solution containing 50 mg/mL of acyclovir. The pH of the reconstituted solution is approximately 11.

Acyclovir sodium is a white, crystalline powder with the molecular formula C8H10N5NaO3 and a molecular weight of 247.19. The maximum solubility in water at 25° C. exceeds 100 mg/mL. At physiologic pH, acyclovir sodium exists as the un-ionized form with a molecular weight of 225 and a maximum solubility in water at 37° C. of 2.5 mg/mL. The pka's of acyclovir are 2.27 and 9.25. The chemical name of acyclovir sodium is 2-amino-1,9-dihydro-9-[(2-hydroxyethoxy)methyl]-6H-purin-6-one monosodium salt.

Dosage and Administration

In some embodiments, acyclovir sodium can be administered according to:
Adults and Adolescents (Aged 12 years and older): 5 mg/kg infused at a constant rate over 1 hour, every 8 hours for 7 days.
Pediatrics (Aged 3 months to 12 years): 10 mg/kg infused at a constant rate over 1 hour, every 8 hours for 7 days.
Adults and Adolescents (Aged 12 years and older): 5 mg/kg infused at a constant rate over 1 hour, every 8 hours for 5 days.
Adults and Adolescents (Aged 12 years and older): 10 mg/kg infused at a constant rate over 1 hour, every 8 hours for 10 days.
Pediatrics (Aged 3 months to 12 years): 20 mg/kg infused at a constant rate over 1 hour, every 8 hours for 10 days.
PMA of at Least 34 Weeks: 20 mg/kg infused at a constant rate over 1 hour, every 8 hours for 21 days.
PMA of Less than 34 Weeks: 20 mg/kg infused at a constant rate over 1 hour, every 12 hours for 21 days.

In neonates with ongoing medical conditions affecting their renal function beyond the effect of prematurity, the doses recommended should be used with caution.
Adults and Adolescents (Aged 12 years and older): 10 mg/kg infused at a constant rate over 1 hour, every 8 hours for 7 days.
Pediatrics (Younger than 12 years): 20 mg/kg infused at a constant rate over 1 hour, every 8 hours for 7 days.
Obese Patients: Obese patients should be dosed at the recommended adult dose using Ideal Body Weight.

In some embodiments, acyclovir sodium can be administered at a dose of 200-3200 mg/day

### Bevacizumab (AVASTIN®)

Bevacizumab is a vascular endothelial growth factor-specific angiogenesis inhibitor indicated for the treatment of:

Metastatic colorectal cancer, with intravenous 5-fluorouracil-based chemotherapy for first- or second-line treatment.

Non-squamous non-small cell lung cancer, with carboplatin and paclitaxel for first line treatment of unresectable, locally advanced, recurrent or metastatic disease.

Metastatic breast cancer, with paclitaxel for treatment of patients who have not received chemotherapy for metastatic HER2-negative breast cancer.

Glioblastoma, as a single agent for patients with progressive disease following prior therapy.

Dosage and Administration

Dosage forms and strengths include: 100 mg/4 mL, single use vial and 400 mg/16 mL, single use vial.

In some embodiments, bevacizumab can be administered according to:
5 mg/kg IV every 2 weeks with bolus-IFL
10 mg/kg IV every 2 weeks with FOLFOX4
15 mg/kg IV every 3 weeks with carboplatin/paclitaxel
10 mg/kg IV every 2 weeks with paclitaxel
10 mg/kg IV every 2 weeks.

### Bromelain

Bromelain belongs to a group of protein digesting enzymes obtained commercially from the fruit or stem of pineapple. Fruit bromelain and stem bromelainare prepared differently and they contain different enzymatic composition. “Bromelain” refers usually to the “stem bromelain.” Bromelain is a mixture of different thiol endopeptidases and other components like phosphatase, glucosidase, peroxidase, cellulase, escharase, and several protease inhibitors. In vitro and in vivo studies demonstrate that bromelain exhibits various fibrinolytic, antiedematous, antithrombotic, and anti-inflammatory activities. Bromelain is considerably absorbable in the body without losing its proteolytic activity and without producing any major side effects. Bromelain accounts for many therapeutic benefits like the treatment of angina pectoris, bronchitis, sinusitis, surgical trauma, and thrombophlebitis, debridement of wounds, and enhanced absorption of drugs, particularly antibiotics. It also relieves osteoarthritis, diarrhea, and various cardiovascular disorders. Bromelain also possesses some anticancerous activities and promotes apoptotic cell death. This paper reviews the important properties and therapeutic applications of bromelain, along with the possible mode of action.

Toxicity of Bromelain

Bromelain has very low toxicity with an LD50 (lethal doses) greater than 10 g/kg in mice, rates, and rabbits. Toxicity tests on dogs, with increasing level of bromelain up to 750 mg/kg administered daily, showed no toxic effects after six months. Dosages of 1500 mg/kg per day when administered to rats showed no carcinogenic or teratogenic effects and did not provoke any alteration in food intake, histology of heart, growth, spleen, kidney, or hematological parameters. Bromelain has been administered to human subjects at doses of 3000 FIP unit/day over a period of ten days without significant changes in blood coagulation parameters. In some embodiments, bromelain can be administered at doses at or below 3000 FIP unit/day.

Dosage and Administration

In some embodiments, bromelain can be administered as an oral dose of 80-1200 mg/day.

### Bromhexine Hydrochloride

Bromhexine is an expectorant/mucolytic agent. The drug is a benzylamine derivative (2-amino3,5-dibromo-N-cyclohexyl N-methylbenzylamine hydrochloride) and also a derivative of vasicine and adhatodic acid, alkaloids obtained from the plant Adhatoda vasica. Following oral administration, bromhexine has increased sputum volume and reduced the viscosity of bronchial secretions in chronic bronchitis patients. The drug has been reported to induce hydrolytic depolymerization of mucoprotein fibers and stimulate activity of the ciliated epithelium. An increase in lysosomal activity facilitated by bromhexine has been postulated. Improvements in pulmonary function in bronchitis patients appear secondary to easier expectoration. An effect of bromhexine on increasing sputum concentrations of various antibiotics (eg, oxytetracycline, erythromycin, ampicillin, amoxicillin) has also been reported. However, some of these effects (exocrine stimulation, increased sputum concentrations) have not been confirmed in some studies. It has been suggested that a metabolite of bromhexine, ambroxol, may contribute to enhanced secretion from exocrine glands during bromhexine administration.

Dosage and Administration

In some embodiments, bromhexine can be administered according to:

Adult Dosage: Bromhexine is usually given orally in a dose of 8 to 16 mg three times daily. At commencement of treatment, it may be necessary to increase the total daily dose up to 48 mg in adults (initially for 7 days).

Pediatric Dosage: Dosing according to body weight: 0.3 mg/kg/day 8 hourly for 7 days then 0.15 mg/kg/day 8 hourly; Dosing according to age: Children 6-12 years: 4 mg 3 times daily Children 2-6 years: 2 mg 3 times daily Children under 2 years: 1 mg 3 times daily

### Chloroquine Phosphate (ARALEN®)

ARALEN, chloroquine phosphate, USP, is a 4-aminoquinoline compound for oral administration. It is a white, odorless, bitter tasting, crystalline substance, freely soluble in water. ARALEN is an antimalarial and amebicidal drug. Chemically, it is 7-chloro-4-[[4-(diethylamino)-1-methylbutyl]amino] quinoline phosphate (1:2).

Each tablet contains 500 mg of chloroquine phosphate USP, equivalent to 300 mg chloroquine base.

Inactive Ingredients: Carnauba Wax, Colloidal Silicon Dioxide, Dibasic Calcium Phosphate, Hydroxypropyl Methylcellulose, Magnesium Stearate, Microcrystalline Cellulose, Polyethylene Glycol, Polysorbate 80, Pregelatinized Starch, Sodium Starch Glycolate, Stearic Acid, Titanium Dioxide.

Clinical Pharmacology

Chloroquine is rapidly and almost completely absorbed from the gastrointestinal tract, and only a small proportion of the administered dose is found in the stools. Approximately 55% of the drug in the plasma is bound to nondiffusible plasma constituents. Excretion of chloroquine is quite slow, but is increased by acidification of the urine. Chloroquine is deposited in the tissues in considerable amounts. In animals, from 200 to 700 times the plasma concentration may be found in the liver, spleen, kidney, and lung; leukocytes also concentrate the drug. The brain and spinal cord, in contrast, contain only 10 to 30 times the amount present in plasma.

Chloroquine undergoes appreciable degradation in the body. The main metabolite is desethylchloroquine, which accounts for one fourth of the total material appearing in the urine; bisdesethylchloroquine, a carboxylic acid derivative, and other metabolic products as yet uncharacterized are found in small amounts. Slightly more than half of the urinary drug products can be accounted for as unchanged chloroquine.

Dosage and Administration

The dosage of chloroquine phosphate is often expressed in terms of equivalent chloroquine base. Each 500 mg tablet of ARALEN contains the equivalent of 300 mg chloroquine base. In infants and children the dosage is preferably calculated by body weight.

In some embodiments, chloroquine phosphate can be administered according to the following:

Adult Dose: 500 mg (=300 mg base) on exactly the same day of each week.

Pediatric Dose: The weekly suppressive dosage is 5 mg calculated as base, per kg of body weight, but should not exceed the adult dose regardless of weight. If circumstances permit, suppressive therapy should begin two weeks prior to exposure. However, failing this in adults, an initial double (loading) dose of 1 g (=600 mg base), or in children 10 mg base/kg may be taken in two divided doses, six hours apart. The suppressive therapy should be continued for eight weeks after leaving the enemic area.

Adults: An initial dose of 1 g (=600 mg base) followed by an additional 500 mg (=300 mg base) after six to eight hours and a single dose of 500 mg (=300 mg base) on each of two consecutive days. This represents a total dose of 2.5 g chloroquine phosphate or 1.5 g base in three days.

The dosage for adults of low body weight and for infants and children should be determined as follows: First dose: 10 mg base per kg (but not exceeding a single dose of 600 mg base). Second dose: (6 hours after first dose) 5 mg base per kg (but not exceeding a single dose of 300 mg base). Third dose: (24 hours after first dose) 5 mg base per kg. Fourth dose: (36 hours after first dose) 5 mg base per kg. For radical cure of vivax and malariae malaria concomitant therapy with an 8-aminoquinoline compound is necessary.

Adults, 1 g (600 mg base) daily for two days, followed by 500 mg (300 mg base) daily for at least two to three weeks. Treatment is usually combined with an effective intestinal amebicide.

### Danoprevir (GANOVO®)

Danoprevir is an orally available 15-membered macrocyclic peptidomimetic inhibitor of NS3/4A HCV protease. It contains acylsulfonamide, fluoroisoindole and tert-butyl carbamate moieties. Danoprevir is a clinical candidate based on its favorable potency profile against multiple HCV genotypes 1-6 and key mutants (GT1b, IC50=0.2-0.4 nM; replicon GT1b, EC50=1.6 nM).

Clinical Trials

Ganovo has recently been evaluated for efficacy and safety in combination with ritonavir for patients infecrted with SARS-CoV-2.

In some embodiments, Ganovo can be administered according to:

Ganovo one tablet (100 mg/tablet) at a time, twice a day, up to 14 days. Ritonavir one tablet(100 mg/tablet) at a time, twice a day, up to 14 days. With or without spray inhalation of interferon, 50 g/time for adults, twice a day up to 14 days.

### Delaviridine Mesylate (RESCRIPTOR®)

RESCRIPTOR Tablets contain delavirdine mesylate, a synthetic non-nucleoside reverse transcriptase inhibitor of the human immunodeficiency virus type 1 (HIV-1). The chemical name of delavirdine mesylate is piperazine, 1-[3-[(1-methyl-ethyl)amino]-2-pyridinyl]-4-[[5-[(methylsulfonyl)amino]-1H-indol-2-yl]carbonyl]-, monomethanesulfonate. Its molecular formula is C22H28N6O3S·CH4O3S, and its molecular weight is 552.68.

Delavirdine mesylate is an odorless white-to-tan crystalline powder. The aqueous solubility of delavirdine free base at 23° C. is 2942 mg/mL at pH 1.0, 295 mg/mL at pH 2.0, and 0.81 mg/mL at pH 7.4. Each RESCRIPTOR Tablets, for oral administration, contains 100 or 200 mg of delavirdine mesylate (henceforth referred to as delavirdine). Inactive ingredients consist of lactose, microcrystalline cellulose, croscarmellose sodium, magnesium stearate, colloidal silicon dioxide, and carnauba wax. In addition, the 100-mg tablet contains Opadry White YS-1-7000-E and the 200-mg tablet contains hydroxypropyl methylcellulose, Opadry White YS-1-18202-A and Pharmaceutical Ink Black.

Dosage and Administration

In some embodiments, delaviridine mesylate can be administered according to:

The recommended dosage for RESCRIPTOR Tablets is 400 mg (four 100-mg or two 200-mg tablets) three times daily. RESCRIPTOR should be used in combination with other appropriate antiretroviral therapy. The complete prescribing information for other antiretroviral agents should be consulted for information on dosage and administration.

The 100-mg RESCRIPTOR Tablets may be dispersed in water prior to consumption. To prepare a dispersion, add four 100-mg RESCRIPTOR Tablets to at least 3 ounces of water, allow to stand for a few minutes, and then stir until a uniform dispersion occurs. The dispersion should be consumed promptly. The glass should be rinsed with water and the rinse swallowed to insure the entire dose is consumed. The 200-mg tablets should be taken as intact tablets, because they are not readily dispersed in water. Note: The 200-mg tablets are approximately one third smaller in size than the 100-mg tablets.

RESCRIPTOR Tablets may be administered with or without food. Patients with achlorhydria should take RESCRIPTOR with an acidic beverage (eg, orange or cranberry juice). However, the effect of an acidic beverage on the absorption of delavirdine in patients with achlorhydria has not been investigated. Patients taking both RESCRIPTOR and antacids should be advised to take them at least one hour apart

In some embodiments, delavirdine mesylate can be administered at a dose of 100-200 mg/day.

### Didanosine (VIDEX®)

VIDEX® (didanosine) is a brand name for didanosine (ddI), a synthetic purine nucleoside analogue active against the Human Immunodeficiency Virus (HIV).

VIDEX Pediatric Powder for Oral Solution is supplied for oral administration in 4- or 8-ounce glass bottles containing 2 or 4 grams of didanosine, respectively. Didanosine is also available as an enteric-coated formulation (VIDEX® EC Delayed-Release Capsules). The chemical name for didanosine is 2′,3′-dideoxyinosine.

Didanosine is a white crystalline powder with the molecular formula C10H12N4O3 and a molecular weight of 236.2. The aqueous solubility of didanosine at 25° C. and pH of approximately 6 is 27.3 mg/mL. Didanosine is unstable in acidic solutions. For example, at pH.

Dosage and Administration

In some embodiments, didanosine can be administered according to:

Adults: The preferred dosing frequency of VIDEX is twice daily because there is more evidence to support the effectiveness of this dosing regimen. Once-daily dosing should be considered only for adult patients whose management requires once-daily dosing of VIDEX

Preferred dosing is 200 mg twice daily for patients greater than or equal to 60 kg.

Preferred dosing is 125 mg twice daily for patients less than 60 kg.

Dosing for patients whose management requires once-daily frequency is 400 mg once daily for patients greater than or equal to 60 kg

Dosing for patients whose management requires once-daily frequency is 250 mg once daily for patients less than 60 kg.

Pediatric Patients: The recommended dose of VIDEX (didanosine) in pediatric patients between 2 weeks and 8 months of age is 100 mg/m2 twice daily, and the recommended VIDEX dose for pediatric patients older than 8 months is 120 mg/m2 twice daily.

In some embodiments, didanosine can be administered at a dose of 125-400 mg/day.

### Dipyridamole (PERSANTINE®)

PERSANTINE® (dipyridamole USP) is a platelet inhibitor chemically described as 2,2′,2″,2′″-[(4,8 Dipiperidinopyrimido[5,4-d]pyrimidine-2,6-diyl)dinitrilo]-tetraethanol.

Dosage and Administration

PERSANTINE tablets are available as round, orange, sugar-coated tablets of 25 mg, 50 mg and 75 mg coded BI/17, BI/18 and BI/19, respectively. They are available in bottles of 100 tablets as indicated: 25 mg Tablets (NDC 0597-0017-01), 50 mg Tablets (NDC 0597-0018-01), and 75 mg Tablets (NDC 0597-0019-01)

In some embodiments, dipyridamole can be administered according to:

75-100 mg four times daily

### Ebastine

Ebastine is a second-generation H1 receptor antagonist that is indicated mainly for allergic rhinitis and chronic idiopathic urticaria. It is available in 10 and 20 mg tablets and as fast-dissolving tablets, as well as in pediatric syrup.

Ebastine is available in different formulations (tablets, fast dissolving tablets and syrup) and commercialized under different brand names around the world,Ebast, Ebatin, Ebatin Fast, Ebatrol, Atmos, Ebet, Ebastel FLAS, Kestine, KestineLIO, KestinLYO, EstivanLYO, Evastel Z, Ebasten (ACI), etc.

Data from over 8,000 patients in more than 40 clinical trials and studies suggest efficacy of ebastine in the treatment of intermittent allergic rhinitis, persistent allergic rhinitis and other indications.

Pharmacokinetics

After oral administration, ebastine undergoes extensive first-pass metabolism by hepatic cytochrome P450 3A4 into its active carboxylic acid metabolite, carebastine. This conversion is practically complete.

In some embodiments, ebastine can be administered according to a recommended flexible daily dose of 10 or 20 mg, depending on disease severity.

### Entecavir (BARACLUDE®)

BARACLUDE is a nucleoside analogue indicated for the treatment of chronic hepatitis B virus infection in adults with evidence of active viral replication and either evidence of persistent elevations in serum aminotransferases (ALT or AST) or histologically active disease.

Dosage and Administration

BARACLUDE is available in 0.5 mg and 1 mg tablets, and 0.05 mg/mL oral solution.

In some embodiments, entecavir can be administered according to:
0.5 mg once daily; or
1 mg once daily.

BARACLUDE should be administered on an empty stomach.

In some embodiments, entecavir can be administered at a dose of 0.5-1 mg/day.

### Famotidine (PEPCID®)

The active ingredient in PEPCID® (famotidine) is a histamine H2-receptor antagonist. Famotidine is N′-(aminosulfonyl)-3-[[[2-[(diaminomethylene)amino]-4-thiazolyl]methyl]thio]propanimidamide. The empirical formula of famotidine is C8H15N702S3 and its molecular weight is 337.43.

Famotidine is a white to pale yellow crystalline compound that is freely soluble in glacial acetic acid, slightly soluble in methanol, very slightly soluble in water, and practically insoluble in ethanol. Each tablet for oral administration contains either 20 mg or 40 mg of famotidine and the following inactive ingredients: hydroxypropyl cellulose, hypromellose, iron oxides, magnesium stearate, microcrystalline cellulose, corn starch, talc, titanium dioxide, and carnauba wax.

Dosage and Administration

In some embodiments, Famotidine can be administered according to:
20 mg once daily before bedtime or twice daily in the morning and before bedtime with or without food.
40 mg once daily before bedtime or twice daily in the morning and before bedtime with or without food.
20 mg every 6 hours; adjusted for patient needs (maximum of up to 160 mg every 6 hours).

### Favipiravir (Avigan)

Favipiravir is investigation for use in the treatment of coronavirus disease 2019 (COVID-19) (See ClinicalTrials.gov). At this time, safety and efficacy have not been established. However, preliminary dosing information based on the available published evidence and ongoing clinical trials is provided (Cai 2020; NIH 2020a; NIH 2020b). Whenever possible, treatment should be given as part of a clinical trial.

Dosage and Administration

In some embodiments, Favipiravir can be administered according to:

Oral: 1,600 mg twice daily on day 1, followed by 600 mg twice daily for a total duration of 7 to 14 days (Cai 2020; NIH 2020a).

Oral: 2.4 g every 8 hours for 2 doses, followed by a dose of 1.2 g 8 hours later on day 1, followed by 1.2 g twice daily for a total duration of 7 to 10 days (NIH 2020b). Ganciclovir sodium (CYTOVENE®-IV)

CYTOVENE-IV is a deoxynucleoside analogue cytomegalovirus (CMV) DNA polymerase inhibitor indicated for the treatment of CMV retinitis in immunocompromised adult patients, including patients with acquired immunodeficiency syndrome and the prevention of CMV disease in adult transplant recipients at risk for CMV disease.

Dosage and Administration

CYTOVENE-IV is administered only intravenously.

In some embodiments, ganciclovir sodium can be administered according to:

Induction: 5 mg/kg (given intravenously at a constant rate over 1 hour) every 12 hours for 14 to 21 days.

Maintenance: 5 mg/kg (given intravenously at a constant-rate over 1 hour) once daily for 7 days per week, or 6 mg/kg once daily for 5 days per week.

Induction: 5 mg/kg (given intravenously at a constant rate over 1 hour) every 12 hours for 7 to 14 days.

Maintenance: 5 mg/kg (given intravenously at a constant-rate over 1 hour) once daily, 7 days per week, or 6 mg/kg once daily, 5 days per week until 100 to 120 days post-transplantation.

In some embodiments, ganciclovir sodium can be administered at a dose of 150-6000 mg/day.

### Hydroxychloroquine Sulfate (PLAQUENIL®)

PLAQUENIL® (hydroxychloroquine sulfate) is a white or practically white, crystalline powder, freely soluble in water; practically insoluble in alcohol, chloroform, and in ether. The chemical name for hydroxychloroquine sulfate is 2-[[4-[(7-Chloro-4-quinolyl) amino]pentyl] ethylamino]ethanol sulfate (1:1).

PLAQUENIL (hydroxychloroquine sulfate) tablets contain 200 mg hydroxychloroquine sulfate, equivalent to 155 mg base, and are for oral administration. Inactive Ingredients: Dibasic calcium phosphate USP, hypromellose USP, magnesium stearate NF, polyethylene glycol 400 NF, polysorbate 80 NF, corn starch, titanium dioxide USP, carnauba wax NF, shellac NF, black iron oxide NF.

Clinical Pharmacology—Pharmacokinetics

Following a single 200 mg oral dose of PLAQUENIL to healthy males, the mean peak blood concentration of hydroxychloroquine was 129.6 ng/mL, reached in 3.26 hours with a half-life of 537 hours (22.4 days). In the same study, the plasma peak concentration was 50.3 ng/mL reached in 3.74 hours with a half-life of 2963 hours (123.5 days). Urine hydroxychloroquine levels were still detectable after 3 months with approximately 10% of the dose excreted as the parent drug. Results following a single dose of a 200 mg tablet versus i.v. infusion (155 mg), demonstrated a half-life of about 40 days and a large volume of distribution. Peak blood concentrations of metabolites were observed at the same time as peak levels of hydroxychloroquine. The mean fraction of the dose absorbed was 0.74. After administration of single 155 mg and 310 mg intravenous doses, peak blood concentrations ranged from 1161 ng/mL to 2436 ng/mL (mean 1918 ng/mL) following the 155 mg infusion and 6 months following the 310 mg infusion. Pharmacokinetic parameters were not significantly different over the therapeutic dose range of 155 mg and 310 mg indicating linear kinetics.

Following chronic oral administration of hydroxychloroquine, significant levels of three metabolites, desethylhydroxychloroquine (DHCQ), desethylchloroquine (DCQ), and bidesethylhydroxychloroquine (BDCQ) have been found in plasma and blood, with DHCQ being the major metabolite. The absorption half-life was approximately 3 to 4 hours and the terminal half-life ranged from 40 to 50 days. The long half-life can be attributed to extensive tissue uptake rather than through decreased excretion. Peak plasma levels of hydroxychloroquine were seen in about 3 to 4 hours. Renal clearance in rheumatoid arthritis (RA) patients taking PLAQUENIL for at least six months seemed to be similar to that of the single dose studies in volunteers, suggesting that no change occurs with chronic dosing. Range for renal clearance of unchanged drug was approximately 16 to 30% and did not correlate with creatinine clearance; therefore, a dosage adjustment is not required for patients with renal impairment. In RA patients, there was large variability as to the fraction of the dose absorbed (i.e. 30 to 100%), and mean hydroxychloroquine levels were significantly higher in patients with less disease activity. Cellular levels of patients on daily hydroxychloroquine have been shown to be higher in mononuclear cells than polymorphonuclear leucocytes.

Dosage and Administration

One PLAQUENIL tablet contains 200 mg of hydroxychloroquine sulfate, which is equivalent to 155 mg base. Take PLAQUENIL with a meal or a glass of milk.

In some embodiments, hydroxychloroquine sulfate can be administered according to:

Adults: 400 mg (310 mg base) once weekly on the same day of each week starting 2 weeks prior to exposure, and continued for 4 weeks after leaving the endemic area.

Weight-based dosing in adults and pediatric patients: 6.5 mg/kg (5 mg/kg base), not to exceed 400 mg (310 mg base), once weekly on the same day of the week starting 2 weeks prior to exposure, and continued for 4 weeks after leaving the endemic area.

Adults: 800 mg (620 mg base) followed by 400 mg (310 mg base) at 6 hours, 24 hours and 48 hours after the initial dose (total 2000 mg hydroxychloroquine sulfate or 1550 mg base).

Weight based dosage in adults and pediatric patients: 13 mg/kg (10 mg/kg base), not to exceed 800 mg (620 mg base) followed by 6.5 mg/kg (5 mg/kg base), not to exceed 400 mg (310 mg base), at 6 hours, 24 hours and 48 hours after the initial dose. PLAQUENIL film-coated tablets cannot be divided, therefore they should not be used to treat patients who weigh less than 31 kg.

Adults: 200 to 400 mg (155 to 310 mg base) daily, administered as a single daily dose or in two divided doses. Doses above 400 mg a day are not recommended. The incidence of retinopathy has been reported to be higher when this maintenance dose is exceeded.

Adults: 400 mg to 600 mg (310 to 465 mg base) daily, administered as a single daily dose or in two divided doses. In a small percentage of patients, side effects may require temporary reduction of the initial dosage. Maintenance adult dosage: When a good response is obtained, the dosage may be reduced by 50 percent and continued at a maintenance level of 200 mg to 400 mg (155 to 310 mg base) daily, administered as a single daily dose or in two divided doses. Do not exceed 600 mg or 6.5 mg/kg (5 mg/kg base) per day, whichever is lower, as the incidence of retinopathy has been reported to be higher when this maintenance dose is exceeded.

### Interferon Alpha (INTRON® A)

INTRON® A (Interferon alfa-2b) for intramuscular, subcutaneous, intralesional, or intravenous Injection is a purified sterile recombinant interferon product.

INTRON A recombinant for Injection has been classified as an alpha interferon and is a water-soluble protein with a molecular weight of 19,271 daltons produced by recombinant DNA techniques. It is obtained from the bacterial fermentation of a strain of Escherichia coli bearing a genetically engineered plasmid containing an interferon alfa2b gene from human leukocytes. The fermentation is carried out in a defined nutrient medium containing the antibiotic tetracycline hydrochloride at a concentration of 5 to 10 mg/L; the presence of this antibiotic is not detectable in the final product. The specific activity of interferon alfa-2b, recombinant is approximately 2.6×108 IU/mg protein as measured by the HPLC assay.

Dosage and Administration

General IMPORTANT: INTRON® A is supplied as 1) Powder for Injection/Reconstitution; 2) Solution for Injection in Vials. Not all dosage forms and strengths are appropriate for some indications. It is important that you carefully read the instructions below for the indication you are treating to ensure you are using an appropriate dosage form and strength.

To enhance the tolerability of INTRON A, injections should be administered in the evening when possible. To reduce the incidence of certain adverse reactions, acetaminophen may be administered at the time of injection. The solution should be allowed to come to room temperature before using

In some embodiments, Ganciclovir can be administered according to:

Dose: 2 million IU/m2 administered intramuscularly or subcutaneously 3 times a week for up to 6 months. Patients with platelet counts of less than 50,000/mm3 should not be administered INTRON A intramuscularly, but instead by subcutaneous administration. Patients who are responding to therapy may benefit from continued treatment.

If severe adverse reactions develop, the dosage should be modified (50% reduction) or therapy should be temporarily withheld until the adverse reactions abate and then resume at 50% (1 MIU/m2 TIW).

Dose: 20 million IU/m2 as an intravenous infusion, over 20 minutes, 5 consecutive days per week, for 4 weeks.

INTRON A should be withheld for severe adverse reactions, including granulocyte counts greater than 250/mm3 but less than 500/mm3 or SGPT/SGOT greater than 5 10× upper limit of normal, until adverse reactions abate. INTRON A treatment should be restarted at 50% of the previous dose.

Maintenance Recommended Dose: The recommended dose of INTRON A for maintenance is 10 million IU/m2 as a subcutaneous injection three times per week for 48 weeks

Dose: 5 million IU subcutaneously three times per week for up to 18 months

Dose: 30 million IU/m2/dose administered subcutaneously or intramuscularly three times a week

INTRON A dose should be reduced by 50% or withheld for severe adverse reactions.

Dose: 3 million IU three times a week (TIW) administered subcutaneously or intramuscularly

If severe adverse reactions develop during INTRON A treatment, the dose should be modified (50% reduction) or therapy should be temporarily discontinued until the adverse reactions abate. If intolerance persists after dose adjustment, INTRON A therapy should be discontinued.

Dose: 30 to 35 million IU per week, administered subcutaneously or intramuscularly, either as 5 million IU daily (QD) or as 10 million IU three times a week (TIW) for 16 weeks.

Dose: 3 million IU/m2 three times a week (TIW) for the first week of therapy followed by dose escalation to 6 million IU/m2 TIW (maximum of 10 million IU TIW) administered subcutaneously for a total duration of 16 to 24 weeks.

If severe adverse reactions or laboratory abnormalities develop during INTRON A therapy, the dose should be modified (50% reduction) or discontinued if appropriate, until the adverse reactions abate. If intolerance persists after dose adjustment, INTRON A therapy should be discontinued.

### Intravenous Immunoglobulin (IVIg)

IVIg is a pool of IgG from thousands of healthy donors, and exposure of individual donors to endemic infectious diseases, vaccines, and ubiquitous microorganisms participates in the production of IgG antibodies against different microorganisms.

Immunotherapy with immune IgG combined with antiviral drugs could provide alternative treatment against COVID-19. These immune IgG antibodies collected from the recovered patients will be specific against COVID-19 by boosting the immune response in newly infected patients. Although a vaccine for COVID-19 is currently not available, the combination of the immune IgG antibodies with antiviral drugs can offer short-term and medium-term solutions against COVID-19.

### Lamivudine (EPIVIR®)

EPIVIR is a nucleoside analogue reverse transcriptase inhibitor indicated in combination with other antiretroviral agents for the treatment of HIV-1 infection.

Dosage and Administration

In some embodiments, EPIVIR is administered according to:

Adults: 300 mg daily, administered as either 150 mg twice daily or 300 mg once daily.

Pediatric Patients Aged 3 Months and Older: Administered either once or twice daily. Dose should be calculated on body weight (kg) and should not exceed 300 mg daily.

In some embodiments, lamivudine can be administered at a dose of 100-300 mg/day.

### Lopinavir/Ritonavir (KALETRA®)

KALETRA is a co-formulation of lopinavir and ritonavir. Lopinavir is an inhibitor of the HIV-1 protease. As co-formulated in KALETRA, ritonavir inhibits the CYP3A-mediated metabolism of lopinavir, thereby providing increased plasma levels of lopinavir. Lopinavir is chemically designated as [1S-[1R*,(R*), 3R*, 4R*]]-N-[4-[[(2,6 dimethylphenoxy)acetyl]amino]-3-hydroxy-5-phenyl-1-(phenylmethyl)pentyl]tetrahydro-alpha (1-methylethyl)-2-oxo-1(2H)-pyrimidineacetamide. Its molecular formula is C37H48N4O5, and its molecular weight is 628.80. Lopinavir is a white to light tan powder. It is freely soluble in methanol and ethanol, soluble in isopropanol and practically insoluble in water.

KALETRA is indicated in combination with other antiretroviral agents for the treatment of HIV1 infection in adults and pediatric patients (14 days and older).

Clinical Pharmacology

Lopinavir is an antiviral drug [see Microbiology (12.4)]. As co-formulated in KALETRA, ritonavir inhibits the CYP3A-mediated metabolism of lopinavir, thereby providing increased plasma levels of lopinavir. 12.3 Pharmacokinetics The pharmacokinetic properties of lopinavir co-administered with ritonavir have been evaluated in healthy adult volunteers and in HIV-1 infected patients; no substantial differences were observed between the two groups. Lopinavir is essentially completely metabolized by CYP3A. Ritonavir inhibits the metabolism of lopinavir, thereby increasing the plasma levels of lopinavir. Across studies, administration of KALETRA 400/100 mg twice daily yields mean steady-state lopinavir plasma concentrations 15- to 20-fold higher than those of ritonavir in HIV-1 infected patients. The plasma levels of ritonavir are less than 7% of those obtained after the ritonavir dose of 600 mg twice daily. The in vitro antiviral EC50 of lopinavir is approximately 10-fold lower than that of ritonavir. Therefore, the antiviral activity of KALETRA is due to lopinavir.

Dosage and Administration

KALETRA tablets may be taken with or without food. The tablets should be swallowed whole and not chewed, broken, or crushed. KALETRA oral solution must be taken with food.

In some embodiments, KALETRA can be administered according to:

Considerations in Determining KALETRA Once Daily vs. Twice Daily Dosing Regimen: (1) KALETRA can be given as once daily or twice daily dosing regimen in patients with less than three lopinavir resistance-associated substitutions; (2) KALETRA must be given as twice daily dosing regimen in patients with three or more resistance-associated substitutions.

KALETRA once daily dosing regimen is not recommended in: (1) Adult patients with three or more of the following lopinavir resistance-associated substitutions: L10F/I/R/V, K20M/N/R, L24I, L33F, M361, 147V, G48V, I54L/T/V, V82A/C/F/S/T, and I84V [see Microbiology (12.4)].; (2) In combination with carbamazepine, phenobarbital, or phenytoin [see Drug Interactions (7.3); (3) In combination with efavirenz, nevirapine, or nelfinavir [see Drug Interactions (7.3) and Clinical Pharmacology (12.3)]; (4) In pregnant women [see Dosage and Administration (2.4), Use in Specific Populations (8.1) and Clinical Pharmacology (12.3)].

The dose of KALETRA must be increased when administered in combination with efavirenz, nevirapine or nelfinavir.

Dosage Forms and Strengths:

Tablets, 200 mg lopinavir, 50 mg ritonavir: Yellow, film-coated, ovaloid, debossed with the “a” logo and the code KA providing 200 mg lopinavir and 50 mg ritonavir.

Tablets, 100 mg lopinavir, 25 mg ritonavir: Pale yellow, film-coated, ovaloid, debossed with the “a” logo and the code KC providing 100 mg lopinavir and 25 mg ritonavir.

Oral Solution: Light yellow to orange colored liquid containing 400 mg lopinavir and 100 mg ritonavir per 5 mL (80 mg lopinavir and 20 mg ritonavir per mL).

In some embodiments, ritonavir can be administered without lopinavir in an oral dose of 100 mg/day.

### Methylprednisolone (MEDROL®)

MEDROL Tablets contain methylprednisolone which is a glucocorticoid. Glucocorticoids are adrenocortical steroids, both naturally occurring and synthetic, which are readily absorbed from the gastrointestinal tract. Methylprednisolone occurs as a white to practically white, odorless, crystalline powder. It is sparingly soluble in alcohol, in dioxane, and in methanol, slightly soluble in acetone, and in chloroform, and very slightly soluble in ether. It is practically insoluble in water. The chemical name for methylprednisolone is pregna-1,4-diene-3,20-dione, 11,17,21 trihydroxy-6-methyl-, (6α,11β)-and the molecular weight is 374.48.

MEDROL Tablets are indicated in the following conditions: 1. Endocrine Disorders Primary or secondary adrenocortical insufficiency (hydrocortisone or cortisone is the first choice; synthetic analogs may be used in conjunction with mineralocorticoids where applicable; in infancy mineralocorticoid supplementation is of particular importance). Congenital adrenal hyperplasia Nonsuppurative thyroiditis Hypercalcemia associated with cancer 2. Rheumatic Disorders As adjunctive therapy for short-term administration (to tide the patient over an acute episode or exacerbation) in: Rheumatoid arthritis, including juvenile rheumatoid arthritis (selected cases may require low-dose maintenance therapy) Ankylosing spondylitis Acute and subacute bursitis Synovitis of osteoarthritis Acute nonspecific tenosynovitis Post-traumatic osteoarthritis Psoriatic arthritis Epicondylitis Acute gouty arthritis 3. Collagen Diseases During an exacerbation or as maintenance therapy in selected cases of: Systemic lupus erythematosus Systemic dermatomyositis (polymyositis) Acute rheumatic carditis 4. Dermatologic Diseases Bullous dermatitis herpetiformis Severe erythema multiforme (Stevens-Johnson syndrome) Severe seborrheic dermatitis Exfoliative dermatitis Mycosis fungoides Pemphigus Severe psoriasis 5. Allergic States Control of severe or incapacitating allergic conditions intractable to adequate trials of conventional treatment: Seasonal or perennial allergic rhinitis Drug hypersensitivity reactions Serum sickness Contact dermatitis Bronchial asthma Atopic dermatitis 6. Ophthalmic Diseases Severe acute and chronic allergic and inflammatory processes involving the eye and its adnexa such as: Allergic corneal marginal ulcers Herpes zoster ophthalmicus Anterior segment inflammation Diffuse posterior uveitis and choroiditis Sympathetic ophthalmia Keratitis Optic neuritis Allergic conjunctivitis Chorioretinitis Iritis and iridocyclitis 7. Respiratory Diseases Symptomatic sarcoidosis Berylliosis Loeffler's syndrome not manageable by other means Fulminating or disseminated pulmonary tuberculosis when used concurrently with appropriate antituberculous chemotherapy Aspiration pneumonitis 8. Hematologic Disorders Idiopathic thrombocytopenic purpura in adults Secondary thrombocytopenia in adults Acquired (autoimmune) hemolytic anemia Erythroblastopenia (RBC anemia) Congenital (erythroid) hypoplastic anemia 9. Neoplastic Diseases For palliative management of: Leukemias and lymphomas in adults Acute leukemia of childhood 10. Edematous States To induce a diuresis or remission of proteinuria in the nephrotic syndrome, without uremia, of the idiopathic type or that due to lupus erythematosus. 11. Gastrointestinal Diseases To tide the patient over a critical period of the disease in: Ulcerative colitis Regional enteritis 12. Nervous System Acute exacerbations of multiple sclerosis 13. Miscellaneous Tuberculous meningitis with subarachnoid block or impending block when used concurrently with appropriate antituberculous chemotherapy. Trichinosis with neurologic or myocardial involvement.

Dosage and Administration

In some embodiments, MEDROL can be administered according to:

The initial dosage of MEDROL Tablets may vary from 4 mg to 48 mg of methylprednisolone per day depending on the specific disease entity being treated. In situations of less severity lower doses will generally suffice while in selected patients higher initial doses may be required. The initial dosage should be maintained or adjusted until a satisfactory response is noted. If after a reasonable period of time there is a lack of satisfactory clinical response, MEDROL should be discontinued and the patient transferred to other appropriate therapy.

It should be emphasized that dosage requirements are variable and must be individualized on the basis of the disease under treatment and the response of the patient. After a favorable response is noted, the proper maintenance dosage should be determined by decreasing the initial drug dosage in small decrements at appropriate time intervals until the lowest dosage which will maintain an adequate clinical response is reached. It should be kept in mind that constant monitoring is needed in regard to drug dosage. Included in the situations which may make dosage adjustments necessary are changes in clinical status secondary to remissions or exacerbations in the disease process, the patient's individual drug responsiveness, and the effect of patient exposure to stressful situations not directly related to the disease entity under treatment; in this latter situation it may be necessary to increase the dosage of MEDROL for a period of time consistent with the patient's condition. If after long-term therapy the drug is to be stopped, it is recommended that it be withdrawn gradually rather than abruptly.

MEDROL Tablets are available in the following strengths and package sizes: 2 mg (white, elliptical, scored, imprinted MEDROL 2); 4 mg (white, elliptical, scored, imprinted MEDROL 4); 8 mg (white, elliptical, scored, imprinted MEDROL 8); 16 mg (white, elliptical, scored, imprinted MEDROL 16); and 32 mg (white, elliptical, scored, imprinted MEDROL 32).

### Nelfinavir Mesylate (VIRACEPT®)

VIRACEPT® (nelfinavir mesylate) is an inhibitor of the human immunodeficiency virus (HIV) protease. VIRACEPT Tablets are available for oral administration as a light blue, capsule-shaped tablet with a clear film coating in 250 mg strength (as nelfinavir free base) and as a white oval tablet with a clear film coating in 625 mg strength (as nelfinavir free base). Each tablet contains the following common inactive ingredients: calcium silicate, crospovidone, magnesium stearate, hypromellose, and triacetin. In addition, the 250 mg tablet contains FD&C blue #2 powder and the 625 mg tablet contains colloidal silicon dioxide. VIRACEPT Oral Powder is available for oral administration in a 50 mg/g strength (as nelfinavir free base) in bottles. The oral powder also contains the following inactive ingredients: microcrystalline cellulose, maltodextrin, dibasic potassium phosphate, crospovidone, hypromellose, aspartame, sucrose palmitate, and natural and artificial flavor. The chemical name for nelfinavir mesylate is [3S-[2(2S*, 3S*), 3α,4aβ,8aβ]]-N-(1,1-dimethylethyl)decahydro-2-[2-hydroxy-3-[(3-hydroxy-2-methylbenzoyl)amino]-4-(phenylthio)butyl]-3-isoquinoline carboxamide monomethanesulfonate (salt) and the molecular weight is 663.90 (567.79 as the free base).

Nelfinavir mesylate is a white to off-white amorphous powder, slightly soluble in water at pH<4 and freely soluble in methanol, ethanol, 2-propanol and propylene glycol.

Dosage and Administration

VIRACEPT is available in 250 mg and 650 mg tablets. VIRACEPT is available in oral powder as 50 mg/g powder containing 50 mg in each gram.

In some embodiments, Nelfinavir can be administered according to:

Oral dosage of 250-625 mg/day.

Adults: The recommended dose is 1250 mg (five 250 mg tablets or two 625 mg tablets) twice daily or 750 mg (three 250 mg tablets) three times daily. VIRACEPT should be taken with a meal. Patients unable to swallow the 250 or 625 mg tablets may dissolve the tablets in a small amount of water. Once dissolved, patients should mix the cloudy liquid well, and consume it immediately. The glass should be rinsed with water and the rinse swallowed to ensure the entire dose is consumed.

Pediatric Patients (2-13 years): In children 2 years of age and older, the recommended oral dose of VIRACEPT Oral Powder or 250 mg tablets is 45 to 55 mg/kg twice daily or 25 to 35 mg/kg three times daily. All doses should be taken with a meal. Doses higher than the adult maximum dose of 2500 mg per day have not been studied in children. For children unable to take tablets, VIRACEPT Oral Powder may be administered. The oral powder may be mixed with a small amount of water, milk, formula, soy formula, soy milk or dietary supplements; once mixed, the entire contents must be consumed in order to obtain the full dose. If the mixture is not consumed immediately, it must be stored under refrigeration, but storage must not exceed 6 hours. Acidic food or juice (e.g., orange juice, apple juice or apple sauce) are not recommended to be used in combination with VIRACEPT, because the combination may result in a bitter taste. VIRACEPT Oral Powder should not be reconstituted with water in its original container.

### Nivolumab (OPDIVO®)

OPDIVO is a programmed death receptor-1 (PD-1) blocking antibody indicated for the treatment of:

Patients with unresectable or metastatic melanoma, as a single agent or in combination with ipilimumab.

Patients with melanoma with lymph node involvement or metastatic disease who have undergone complete resection, in the adjuvant setting.

Patients with metastatic non-small cell lung cancer and progression on or after platinum-based chemotherapy. Patients with EGFR or ALK genomic tumor aberrations should have disease progression on FDA-approved therapy for these aberrations prior to receiving OPDIVO.

Patients with metastatic small cell lung cancer with progression after platinum-based chemotherapy and at least one other line of therapy.

Patients with advanced renal cell carcinoma who have received prior antiangiogenic therapy.

Patients with intermediate or poor risk, previously untreated advanced renal cell carcinoma, in combination with ipilimumab.

Adult patients with classical Hodgkin lymphoma that has relapsed or progressed after: autologous hematopoietic stem cell transplantation (HSCT) and brentuximab vedotin, or or more lines of systemic therapy that includes autologous HSCT.

Patients with recurrent or metastatic squamous cell carcinoma of the head and neck with disease progression on or after a platinum-based therapy.

Patients with locally advanced or metastatic urothelial carcinoma who: have disease progression during or following platinum-containing chemotherapy; have disease progression within 12 months of neoadjuvant or adjuvant treatment with platinum-containing chemotherapy.

Adult and pediatric (12 years and older) patients with microsatellite instability-high (MSI-H) or mismatch repair deficient (dMMR) metastatic colorectal cancer that has progressed following treatment with a fluoropyrimidine, oxaliplatin, and irinotecan, as a single agent or in combination with ipilimumab.

Patients with hepatocellular carcinoma who have been previously treated with sorafenib.

Dosage and Administration

Administer OPDIVO as an intravenous infusion over 30 minutes.

In some embodiments, nivolumab can be administered according to:

OPDIVO 240 mg every 2 weeks or 480 mg every 4 weeks.

OPDIVO 1 mg/kg, followed by ipilimumab on the same day, every 3 weeks for 4 doses, then OPDIVO 240 mg every 2 weeks or 480 mg every 4 weeks.

OPDIVO 240 mg every 2 weeks.

OPDIVO 3 mg/kg followed by ipilimumab 1 mg/kg on the same day every 3 weeks for 4 doses, then OPDIVO 240 mg every 2 weeks or 480 mg every 4 weeks.

Adult and pediatric patients >/=40 kg: OPDIVO 240 mg every 2 weeks or 480 mg every 4 weeks.

Pediatric patients <40 kg: OPDIVO 3 mg/kg every 2 weeks.

Adult and pediatric patients >/=40 kg: OPDIVO 3 mg/kg followed by ipilimumab 1 mg/kg on the same day every 3 weeks for 4 doses, then OPDIVO 240 mg every 2 weeks or 480 mg every 4 weeks.

Pediatric patients <40 kg: OPDIVO 3 mg/kg followed by ipilimumab 1 mg/kg on the same day every 3 weeks for 4 doses, then OPDIVO 3 mg/kg every 2 weeks.

### Niclosamide (NICLOCIDE)

Niclosamide is an antihelminth used against tapeworm infections. It may act by the uncoupling of the electron transport chain to ATP synthase. The disturbance of this crucial metabolic pathway prevents creation of adenosine tri-phosphate (ATP), an essential molecule that supplies energy for metabolism. Niclosamide works by killing tapeworms on contact. Adult worms (but not ova) are rapidly killed, presumably due to uncoupling of oxidative phosphorylation or stimulation of ATPase activity. The killed worms are then passed in the stool or sometimes destroyed in the intestine. Niclosamide may work as a molluscicide by binding to and damaging DNA. Niclosamide is used for the treatment of tapeworm and intestinal fluke infections: Taenia saginata (Beef Tapeworm), Taenia solium (Pork Tapeworm), Diphyllobothrium latum (Fish Tapeworm), Fasciolopsis buski (large intestinal fluke). Niclosamide is also used as a molluscicide in the control of schistosomiasis. Niclosamide was marketed under the trade name Niclocide, now discontinued.

Niclosamide has been found found to be effective against various viral infections with nanomolar to micromolar potency such as SARS-CoV, MERS-CoV, ZIKV, HCV, and human adenovirus, indicating its potential as an antiviral agent.

Dosage and Administration

In some embodiments, niclosamide can be administered according to:

Adults—2 grams as a single dose (oral administration). Treatment may be repeated in seven days if needed.

Recombinant human ACE2 Clinical Trials

Recombinant human ACE2 has recently been evaluated for efficacy and safety in patients infected with SARS-CoV-2.

In some embodiments, recombinant human ACE2 can be administered according to:

0.4 mg/kg rhACE2 IV BID for 7 days.

### Remdesivir

Remdesivir is an investigational nucleotide analog with broad-spectrum antiviral activity. Remdesivir has demonstrated in vitro and in vivo activity in animal models against the viral pathogens MERS and SARS, which are also coronaviruses and are structurally similar to COVID-19. The limited preclinical data on remdesivir in MERS and SARS indicate that remdesivir may have potential activity against COVID-19.

Dosage and Administration

In some embodiments, Remdesivir can be administered according to:

Adults 200 mg IV on day 1 then 100 mg IV once daily for 4 to 9 days is being evaluated in multi-center randomized trials.

Children and Adolescents 12 to 18 years weighing 40 kg or more. Efficacy in pediatric patients and optimal dosing are not established. 200 mg IV on day 1 then 100 mg IV for 9 days is being evaluated for compassionate use. A dosing regimen of 5 mg/kg/dose (Max: 200 mg/dose) IV once daily on day 1, followed by 2.5 mg/kg/dose (Max: 100 mg/dose) IV once daily was used in 41 pediatric patients (including 2 neonates) who received remdesivir in a phase 3 Ebola study. Optimal duration of therapy for COVID-19 is unknown; a 5- to 10-day course is being studied in adult patients.

Children and Adolescents 12 to 18 years weighing less than 40 kg. Efficacy in pediatric patients and optimal dosing are not established. A dosing regimen of 5 mg/kg/dose (Max: 200 mg/dose) IV once daily on day 1, followed by 2.5 mg/kg/dose (Max: 100 mg/dose) IV once daily was used in 41 pediatric patients (including 2 neonates) who received remdesivir in a phase 3 Ebola study. Optimal duration of therapy for COVID-19 is unknown; a 5- to 10-day course is being studied in adult patients.

Infants and Children 1 month to 11 years. Efficacy in pediatric patients and optimal dosing are not established. A dosing regimen of 5 mg/kg/dose (Max: 200 mg/dose) IV once daily on day 1, followed by 2.5 mg/kg/dose (Max: 100 mg/dose) IV once daily was used in 41 pediatric patients (including 2 neonates) who received remdesivir in a phase 3 Ebola study. Optimal duration of therapy for COVID-19 is unknown; a 5- to 10-day course is being studied in adult patients.

Maximum Dosage Limits Adults. Safety and efficacy have not been established; however, investigational doses of 200 mg IV on day 1, followed by 100 mg IV once daily have been used.

Geriatric Safety and efficacy have not been established; however, investigational doses of 200 mg IV on day 1, followed by 100 mg IV once daily have been used.

Adolescents Safety and efficacy have not been established; however, investigational doses of 5 mg/kg/dose (Max: 200 mg) IV on day 1, followed by 2.5 mg/kg/dose IV once daily (Max: 100 mg) have been used.

Children Safety and efficacy have not been established; however, investigational doses of 5 mg/kg/dose (Max: 200 mg) IV on day 1, followed by 2.5 mg/kg/dose IV once daily (Max: 100 mg) have been used.

Infants Safety and efficacy have not been established; however, investigational doses of 5 mg/kg/dose IV on day 1, followed by 2.5 mg/kg/dose IV once daily have been used.

Neonates Safety and efficacy have not been established.

### Remestemcel-L (PROCHYMAL®)

PROCHYMAL (remestemcel-L, human mesenchymal stem cells [hMSCs] for intravenous infusion) is a liquid cell suspension of ex-vivo cultured adult human mesenchymal stem cells intended for intravenous infusion. The mesenchymal stem cells are derived from the bone marrow of unrelated and human leukocyte antigen (HLA)-unmatched healthy adult donors. Patient-specific blood type or HLA matching is not required for the administration of hMSCs due to the product's low immunogenic profile.

The hMSCs are undifferentiated stem cells of mesodermal origin. They are primary cells that have not been genetically manipulated or immortalized during the manufacturing process. The hMSCs are manufactured under aseptic conditions in a process that involves isolation and culture expansion. PROCHYMAL is provided as a frozen cell suspension in a cryogenic bag. The cells should be thawed and diluted prior to intravenous administration.

PROCHYMAL is indicated for in the management of acute Graft versus Host Disease (aGvHD) in pediatric patients. Acute GvHD should be refractory to treatment with systemic corticosteroid therapy and/or other immunosuppressive agents. Prochymal may be used for Grades C and D of the disease in any organ. Prochymal may also be used in the management of Grade B aGvHD involving any visceral organ, including the GI tract and the liver, but excluding skin. Approval with conditions is based on clinical study of severe refractory aGvHD patients that demonstrated a clinically significant Overall Response of their aGvHD 28 days following start of PROCHYMAL.

Dosage and Administration

In some embodiments, PROCHYMAL can be administered according to:

Dosing of PROCHYMAL is based on body weight. The recommended dose of PROCHYMAL is 2×10\hat{ }6 hMSC/kg (actual body weight) administered intravenously at a controlled rate of 4-6 mL/minute by infusion pump for patients weighing 35 kg and over. For patients under 35 kg in weight, PROCHYMAL should be infused over the course of 60 minutes. Patients should be treated with PROCHYMAL twice per week for 4 consecutive weeks. Infusions should be administered at least 3 days apart. A therapy assessment should be performed after the fourth week of treatment as described below to determine whether continued treatment is warranted.

A therapy assessment should be performed after initial treatment is completed to determine whether continued treatment is warranted. Continued treatment can be initiated if the patient has achieved a response to treatment without safety issues. The recommended continued treatment dosing of PROCHYMAL is 2×106 hMSC/kg administered once a week for 4 weeks.

### Saquinavir Mesylate (INVIRASE®)

INVIRASE is an HIV-1 protease inhibitor indicated for the treatment of HIV1 infection in combination with ritonavir and other antiretroviral agents in adults (over the age of 16 years).

Dosage and Administration

INVIRASE comes in 200 mg capsules and 500 mg film-coated tablets

In some embodiments, INVIRASE can be administered according to:

INVIRASE must be administered in combination with ritonavir.

Adults (over the age of 16 years): INVIRASE 1000 mg twice daily (5×200 mg capsules or 2×500 mg tablets) in combination with ritonavir 100 mg twice daily.

INVIRASE and ritonavir should be taken within 2 hours after a meal.

In certain embodiments, saquinavir mesylate can be administered in a dose of 1000-2000 mg/day.

### Sildenafil Citrate (VIAGRA®)

VIAGRA is a phosphodiesterase-5 (PDE5) inhibitor indicated for the treatment of erectile dysfunction (ED).

Clinical Pharmacology

The physiologic mechanism of erection of the penis involves release of nitric oxide (NO) in the corpus cavernosum during sexual stimulation. NO then activates the enzyme guanylate cyclase, which results in increased levels of cyclic guanosine monophosphate (cGMP), producing smooth muscle relaxation in the corpus cavernosum and allowing inflow of blood.

Sildenafil enhances the effect of NO by inhibiting phosphodiesterase type 5 (PDE5), which is responsible for degradation of cGMP in the corpus cavernosum. Sildenafil has no direct relaxant effect on isolated human corpus cavernosum. When sexual stimulation causes local release of NO, inhibition of PDE5 by sildenafil causes increased levels of cGMP in the corpus cavernosum, resulting in smooth muscle relaxation and inflow of blood to the corpus cavernosum. Sildenafil at recommended doses has no effect in the absence of sexual stimulation.

Studies in vitro have shown that sildenafil is selective for PDE5. Its effect is more potent on PDE5 than on other known phosphodiesterases (10-fold for PDE6, >80-fold for PDE1, >700-fold for PDE2, PDE3, PDE4, PDE7, PDE8, PDE9, PDE10, and PDE11). Sildenafil is approximately 4,000-fold more selective for PDE5 compared to PDE3. PDE3 is involved in control of cardiac contractility. Sildenafil is only about 10-fold as potent for PDE5 compared to PDE6, an enzyme found in the retina which is involved in the phototransduction pathway of the retina. This lower selectivity is thought to be the basis for abnormalities related to color vision [see Clinical Pharmacology (12.2)].

In addition to human corpus cavernosum smooth muscle, PDE5 is also found in other tissues including platelets, vascular and visceral smooth muscle, and skeletal muscle, brain, heart, liver, kidney, lung, pancreas, prostate, bladder, testis, and seminal vesicle. The inhibition of PDE5 in some of these tissues by sildenafil may be the basis for the enhanced platelet anti-aggregatory activity of NO observed in vitro, an inhibition of platelet thrombus formation in vivo and peripheral arterial-venous dilatation in vivo.

Dosage and Administration

In some embodiments, VIAGRA can be administered according to:

VIAGRA is supplied as blue, film-coated, rounded-diamond-shaped tablets containing sildenafil citrate equivalent to 25 mg, 50 mg, or 100 mg of sildenafil. Tablets are debossed with PFIZER on one side and VGR25, VGR50 or VGR100 on the other to indicate the dosage strengths.

Pharmacokinetics

VIAGRA is rapidly absorbed after oral administration, with a mean absolute bioavailability of 41% (range 25-63%). The pharmacokinetics of sildenafil are dose-proportional over the recommended dose range. It is eliminated predominantly by hepatic metabolism (mainly CYP3A4) and is converted to an active metabolite with properties similar to the parent, sildenafil. Both sildenafil and the metabolite have terminal half lives of about 4 hours.

VIAGRA is rapidly absorbed. Maximum observed plasma concentrations are reached within 30 to 120 minutes (median 60 minutes) of oral dosing in the fasted state. When VIAGRA is taken with a high fat meal, the rate of absorption is reduced, with a mean delay in Tmax of 60 minutes and a mean reduction in Cmax of 29%. The mean steady state volume of distribution (Vss) for sildenafil is 105 L, indicating distribution into the tissues. Sildenafil and its major circulating N-desmethyl metabolite are both approximately 96% bound to plasma proteins. Protein binding is independent of total drug concentrations. Based upon measurements of sildenafil in semen of healthy volunteers 90 minutes after dosing, less than 0.001% of the administered dose may appear in the semen of patients.

Sildenafil is cleared predominantly by the CYP3A4 (major route) and CYP2C9 (minor route) hepatic microsomal isoenzymes. The major circulating metabolite results from N-desmethylation of sildenafil, and is itself further metabolized. This metabolite has a PDE selectivity profile similar to sildenafil and an in vitro potency for PDE5 approximately 50% of the parent drug. Plasma concentrations of this metabolite are approximately 40% of those seen for sildenafil, so that the metabolite accounts for about 20% of sildenafil's pharmacologic effects. After either oral or intravenous administration, sildenafil is excreted as metabolites predominantly in the feces (approximately 80% of administered oral dose) and to a lesser extent in the urine (approximately 13% of the administered oral dose). Similar values for pharmacokinetic parameters were seen in normal volunteers and in the patient population, using a population pharmacokinetic approach.

### Stavudine (ZERIT®)

ZERIT® is the brand name for stavudine (d4T), a synthetic thymidine nucleoside analogue, active against the human immunodeficiency virus (HIV). ZERIT (stavudine) Capsules are supplied for oral administration in strengths of 15, 20, 30, and 40 mg of stavudine. Each capsule also contains inactive ingredients microcrystalline cellulose, sodium starch glycolate, lactose, and magnesium stearate. The hard gelatin shell consists of gelatin, titanium dioxide, and iron oxides. The capsules are printed with edible inks.

ZERIT (stavudine) for Oral Solution is supplied as a dye-free, fruit-flavored powder in bottles with child-resistant closures providing 200 mL of a 1 mg/mL stavudine solution upon constitution with water per label instructions. The powder for oral solution contains the following inactive ingredients: methylparaben, propylparaben, sodium carboxymethylcellulose, sucrose, and antifoaming and flavoring agents. The chemical name for stavudine is 2′,3′-didehydro-3′-deoxythymidine.

Dosage and Administration

The interval between doses of ZERIT (stavudine) should be 12 hours. ZERIT may be taken with or without food.

In some embodiments, stavudine can be administered according to:

Adults: The recommended dose based on body weight is as follows: 40 mg twice daily for patients >60 kg. 30 mg twice daily for patients <60 kg.

Pediatrics: The recommended dose for newborns from birth to 13 days old is 0.5 mg/kg/dose given every 12 hours (see CLINICAL PHARMACOLOGY). The recommended dose for pediatric patients at least 14 days old and weighing less than 30 kg is 1 mg/kg/dose, given every 12 hours. Pediatric patients weighing 30 kg or greater should receive the recommended adult dosage.

### Tenofovir Disoproxil Fumarate (VIREAD®)

VIREAD is a nucleotide analog HIV-1 reverse transcriptase inhibitor and an HBV reverse transcriptase inhibitor. VIREAD is indicated in combination with other antiretroviral agents for the treatment of HIV-1 infection in adults and pediatric patients 2 years of age and older. VIREAD is indicated for the treatment of chronic hepatitis B in adults and pediatric patients 12 years of age and older.

Dosage and Administration

VIREAD comes in tablets of 150, 200, 250 and 300 mg, and oral powder of 40 mg per 1 g of oral powder)

In some embodiments, VIREAD can be administered according to:

300 mg once daily taken orally without regard to food.

Tablets: for pediatric patients weighing greater than or equal to 17 kg who can swallow an intact tablet, one VIREAD tablet (150, 200, 250 or 300 mg based on body weight) once daily taken orally without regard to food.

Oral powder: 8 mg/kg VIREAD oral powder (up to a maximum of 300 mg) once daily with food.

300 mg every 7 days.

In some embodiments, tenofovir disoproxil fumarate can be administered at a dose of 150-300 mg/day.

### Thymosin (ADAXIN™)

ADAXIN™ thymosin alpha 1 (thymalfasin) for subcutaneous injection is a purified sterile lyophilized preparation of chemically synthesized thymosin alpha 1 identical to human thymosin alpha 1. Thymosin alpha 1 is an acetylated polypeptide with the following sequence: Ac-Ser-Asp-Ala-Ala-Val-Asp-Thr-Ser-Ser-Glu-Ile-Thr-Thr-Lys-Asp-Leu-Lys-Glu-Lys-Lys-Glu-Val-Val-Glu-Glu-Ala-Glu-Asn-OH, and having a molecular weight of 3,108 daltons. The lyophilized preparation contains 1.6 mg thymosin alpha 1, 50 mg mannitol, and sodium phosphate buffer to adjust the pH to 6.8.

Dosage and Administration

Product for Injection: Prior to administration, the lyophilized powder is to be reconstituted with 1 ml of the provided diluent (sterile water for injection). After reconstitution, the final concentration of ZADAXIN (thymalfasin) is 1.6 mg/ml.

ZADAXIN (thymalfasin) is intended for subcutaneous injection and should not be given intravenously. It should be reconstituted with 1.0 ml of the diluent provided, which consists of 1.0 ml Sterile Water for Injection, immediately prior to use. At the discretion of the physician, the patient may be taught to self-administer the medication.

In some embodiments, ZADAXIN can be administered according to:

1.6 mg (900 μg/m2) administered subcutaneously twice a week for 6 to 12 months. Patients weighing less than 40 kg should receive a ZADAXIN (thymalfasin) dose of 40 μg/kg.

1.6 mg (900 μg/m2) administered subcutaneously using various schedules for 6 months or given between chemotherapy cycles for the duration of treatment.

### Thalidomide

THALOMID in combination with dexamethasone is indicated for the treatment of patients with newly diagnosed multiple myeloma (MM).

THALOMID is indicated for the acute treatment of the cutaneous manifestations of moderate to severe erythema nodosum leprosum (ENL). THALOMID is not indicated as monotherapy for such ENL treatment in the presence of moderate to severe neuritis. THALOMID is also indicated as maintenance therapy for prevention and suppression of the cutaneous manifestations of ENL recurrence.

Dosage and Administration

THALOMID 50 mg, 100 mg, 150 mg and 200 mg capsules will be supplied through the THALOMID REMSTM program.

THALOMID is available in the following capsule strengths: 50 mg capsules [white opaque], imprinted “Celgene/50 mg” with a “Do Not Get Pregnant” logo. 100 mg capsules [tan], imprinted “Celgene/100 mg” with a “Do Not Get Pregnant” logo. 150 mg capsules [tan and blue], imprinted “Celgene/150 mg” with a “Do Not Get Pregnant” logo 200 mg capsule [blue], imprinted “Celgene/200 mg” with a “Do not Get Pregnant” logo.

In some embodiments, THALIDOMID can be administered according to:

200 mg orally once daily. The recommended dose of dexamethasone is 40 mg/day on days 1-4, 9-12, and 17-20 every 28 days.

ENL

100 to 300 mg/day.

400 mg/day.

### Umifenovir (Arbidol)

Umifenovir is an indole-based, hydrophobic, dual-acting direct antiviral/host-targeting agent used for the treatment and prophylaxis of influenza and other respiratory infections. Umifenovir's ability to exert antiviral effects through multiple pathways has resulted in considerable investigation into its use for a variety of enveloped and non-enveloped RNA and DNA viruses, including Flavivirus, Zika virus, foot-and-mouth disease, Lassa virus, Ebola virus, herpes simplex, hepatitis B and C viruses, chikungunya virus, reovirus, Hantaan virus, and coxsackie virus B5. This dual activity may also confer additional protection against viral resistance, as the development of resistance to umifenovir does not appear to be significant.

Umifenovir is currently being investigated as a potential treatment and prophylactic agent for COVID-19 caused by SARS-CoV2 infections in combination with both currently available and investigational HIV therapies.

Dosage and Administration

In some embodiments, Umifenovir can be administered according to:

800 mg/day (2 capsules q.i.d.) for 5 days.

200 mg/day (2 capsules q.d.) for 10 days.

Zanamivir (RELENZA®)

RELENZA® (zanamivir) Inhalation Powder is indicated for treatment of uncomplicated acute illness due to influenza A and B virus in adults and pediatric patients aged 7 years and older who have been symptomatic for no more than 2 days.

RELENZA is indicated for prophylaxis of influenza in adults and pediatric patients aged 5 years and older.

Dosage and Administration

Blister for oral inhalation: 5 mg. Four 5-mg blisters of powder on a ROTADISK for oral inhalation via DISKHALER. Packaged in carton containing 5 ROTADISKs (total of 10 doses) and 1 DISKHALER inhalation device.

In some embodiments, RELENZA can be administered according to:
10 mg twice daily (approximately 12 hours apart) for 5 days. Two doses should be taken on the first day of treatment whenever possible provided there is at least 2 hours between doses. On subsequent days, doses should be about 12 hours apart (e.g., morning and evening) at approximately the same time each day. The safety and efficacy of repeated treatment courses have not been studied.
10 mg once daily for 10 days. The dose should be administered at approximately the same time each day. There are no data on the effectiveness of prophylaxis with RELENZA in a household setting when initiated more than 1.5 days after the onset of signs or symptoms in the index case.
10 mg once daily for 28 days. The dose should be administered at approximately the same time each day. There are no data on the effectiveness of prophylaxis with RELENZA in a community outbreak when initiated more than 5 days after the outbreak was identified in the community. The safety and effectiveness of prophylaxis with RELENZA have not been evaluated for longer than 28 days' duration.

In some embodiments, zanamivir can be administered at a dose of 10-20 mg/day.

### Zidovudine (RETROVIR®)

RETROVIR, a nucleoside reverse transcriptase inhibitor, is indicated in combination with 18 other antiretroviral agents for the treatment of HIV-1 infection.

Dosage and Administration

RETROVIR Tablets 300 mg (biconvex, white, round, film-coated) containing 300 mg zidovudine, one side engraved “GX CW3” and “300” on the other side.

RETROVIR Capsules 100 mg (white, opaque cap and body) containing 100 mg zidovudine and printed with “Wellcome” and unicorn logo on cap and “Y9C” and “100” on body.

RETROVIR Syrup (colorless to pale yellow, strawberry-flavored) containing 50 mg zidovudine in each teaspoonful (5 mL).

In some embodiments, RETROVIR can be administered according to:

Adults: 600 mg/day in divided doses with other antiretroviral agents.

Pediatric patients (6 weeks to <18 years of age): Dosage should be calculated based on body weight not to exceed adult dose.

Maternal Dosing: 100 mg orally 5 times per day until the start of labor. During labor and delivery, intravenous RETROVIR should be administered at 2 mg/kg (total body weight) over 1 hour followed by a continuous intravenous infusion of 1 mg/kg/hour (total body weight) until clamping of the umbilical cord.

Neonatal Dosing: 2 mg/kg orally every 6 hours starting within 12 hours after birth and continuing through 6 weeks of age. Neonates unable to receive oral dosing may be administered RETROVIR intravenously at 1.5 mg/kg, infused over 30 minutes, every 6 hours.

In some embodiments, zidovudine can be administered at a dose of 1-40 mg/day.

### Exemplification

### Example 1: COVID-19 Treatment

The present Example describes treatment of patients suffering from COVID-19 with an embodiment of a therapy as provided herein. In particular, the present Example utilizes a six component embodiment of a combination therapy previously described in International Publication WO 2014/169221A2 (the contents of which are hereby incorporated by reference). In the present Example, NED-260 refers to a combination of therapeutically effective amounts of agents comprising alpha-lipoic acid, curcumin, genistein, melatonin, metformin, and naltrexone. In the present Example, patients receiving treatment will be administered a combination of agents at the following doses:

R-Alpha-Lipoic Acid Sodium Salt: Dose 600 mg twice daily; after breakfast and dinner.
Curcumin: 500 mg three times daily; after meals.
Genistein: 250 mg twice daily; after breakfast and dinner.
Melatonin: 10 mg daily; at bedtime.
Metformin: 500 mg twice daily orally; after breakfast and dinner.
Naltrexone: 3.5 mg/day orally; at bedtime.

Study Objectives, Population, and Number of Patients

The present example comprises a two part randomized controlled study of efficacy of the provided embodiment of the present disclosure in the treatment of patients with COVID-19 who are hospitalized or home quarantined or in step down units treated for 28 days.

In Part 1 of the study, the provided embodiment of the present disclosure is administered to hospitalized patients that have COVID-19, but are not considered severe cases (n=10). The primary objective of Part 1 is to assess safety and tolerability in the patient cohort. The secondary objectives of Part 1 are to assess (1) decrease in length of stay in hospital, and (2) decrease in ICU admissions, among the patient cohort.

In Part 2 of the study, the provided embodiment of the present disclosure is administered to eight groups (A-H):
Group A (n=150): Hospitalized patients with COVID-19 with high risk for decompensation with oxygen saturation >94%
Group B (n=150): Group A Best Supportive Care
Group C (n=150): Hospitalized patients with COVID-19 with low risk for decompensation with oxygen saturation >94%
Group D (n=150): Group B Best Supportive Care
Group E (n=150): Non-hospitalized, patients who present to the ER with COVID-19 positive by PCR with high risk for decompensation
Group F (n=150): Group E Best Supportive Care
Group G (n=150): Hospitalized patients with COVID-19 and an oxygen saturation <94%
Group H (n=150): Group G Best Supportive Care

The primary objective of Part 2 is to assess prevention of advancement to ICU admission due to serious disease progression due to SARS-CoV-2 infection (COVID-19) due to cytokine storm. The secondary objectives of Part 2 are to (1) assess safety and tolerability, (2) decrease length of stay in hospital, (3) decrease in rate of ICU admissions, and (4) overall survival.

Inclusion Criteria For All Groups
1. Positive test for SARS-CoV2 infection by PCR, or other standardized acceptable test.
2. Signed informed consent.
3. Subjects >18 years of age.
4. Able to comply with 4 separate daily oral dosing times (once each at breakfast, lunch, dinner, and before bed) including 16 pills daily and not have gastrointestinal disorders that would prevent/compromise the ingestion of NED-260 study drugs.
5. Patients must be able to orally swallow medications.
6. All oxygen saturations will be determined on room air, unless patient is chronically on oxygen, which then will be continued.
7. Admitted as an inpatient or evaluated in the emergency room and discharged to home or other facility.
8. Acceptable organ function as defined below:
A. Absolute neutrophil count >1,500 cells/μl.
B. Platelets >100,000 cells/μl.
C. Hemoglobin ≥9.0 g/dL (may be transfused to be eligible unless due to active bleeding).
D. AST/ALT≤3 times the upper limit of normal.
E. Serum creatinine <2×the upper limit of normal (ULN) or if higher than the institutional normal range, calculated creatinine clearance (CrCl) must be ≥50 mL/min/1.73 m2 (e.g., by Cockcroft-Gault formula); actual body weight must be used for CrCl unless body mass index (BMI) is >30 kg/m2, in which case, lean body weight must be utilized.

Exclusion Criteria
1. Concurrent use of invasive mechanical ventilation (patients receiving non-invasive mechanical ventilation [CPAP, BiPap, HHFNC] are eligible).
2. Concurrent use of vasopressor or inotropic medications
3. Patients who are actively being considered for a study that would potentially exclude concurrent enrollment on this study.
4. Patients actively receiving an investigational agent in the context of a clinical research study.
5. Multi-organ failure as determined by primary treating team
6. Major surgery (excluding minor procedures, e.g., placement of vascular access, gastrointestinal/biliary stent, biopsy)<4 weeks prior to planned first day.
7. Participation in any other clinical investigation using an experimental drug within 4 weeks of the first dose of any component of NED-260.
8. NOTE that patients with diabetes will be allowed on study but will require a physician-Sponsor consult regarding reconciled and optimal metformin dosing (see metformin prescribing information).
9. Women who are pregnant or breastfeeding.
10. Known contraindication or drug-interaction between active co-medication and any NED-260 component that in opinion of investigator will interfere with co-medication or NED-260 absorption, metabolism, or clinical activity.
11. Known allergy or intolerance to any NED-260 component.
12. Sponsor reserves right to exclude any subject from the study on basis of pre-study medical histories, physical examination findings, clinical laboratory results, prior medications, or other entrance criteria.
13. Prisoners

Study Design

Based on the 6-component regimen of NED-260 that utilizes relatively low/safe doses of each component, a standard dose escalation strategy is not possible. In order to evaluate safety in the population before expansion to a large cohort, Part 1 is an open label single arm study to test NED in 10 patients hospitalized, non-severe patients with COVID-19 with risk factors for decompensation prior to opening enrollment into Part 2, a randomized, parallel open label study of patients randomized to a treatment arm or Best Supportive Care.

All subjects will be followed for safety and toxicity throughout the study and for >28 days following NED-260 discontinuation.

All COVID-19-positive patients will require initial EKG, chest imaging, labs, and review of medical history.

Patients will be assigned to Group A/B if: hospitalized and oxygen >94% AND they have at least one of the following high-risk conditions:
Age >60 OR
BMI>30 OR
Diabetes (HgbA1c>8.0) OR
Chronic heart disease/HTN OR
Chronic lung disease OR
Immunosuppressed (includes cancer treatment within 1 year, the use of immunosuppressive drugs (biologics, chronic prednisone >20 mg daily, solid organ transplant, bone marrow transplantation, HIV/AIDS (regardless of CD4 count), leukemia, lymphoma, SLE, and vasculitis)
Patients will be assigned to Group C/D if: hospitalized and oxygen >94% AND they have NONE of the high-risk criteria listed above
Patients will be assigned to Group E/F if non hospitalized and they have any risk factor listed for Group A/B
Patients will be assigned to Group G/H if they are hospitalized and oxygen saturation is <94%

Statistical Analysis

Clinical endpoints include labs, chest imaging, and vital signed with pulse oximetry. Additional endpoints include total number of hospitalized days, total number of days to full recovery, 02 dependency, NIV, intubation, total number of ICU days, need for mechanical ventilation, duration of mechanical ventilation, hospital discharge location, and death.

Safety endpoints employed will include but not necessarily be limited to number and % of subjects experiencing: adverse events of any grade; grade 3-5 adverse events; adverse events of any grade considered by investigator to be at least possibly related to NED-260; grade 3-5 adverse events considered by investigator to be at least possibly related to NED-260; serious adverse events requiring study discontinuation; NED-260 discontinuations or suspensions; SAEs at least possibly related to NED-260.

Quality of Life (QOL) assessments may include but not necessarily be limited to the Patient Global Impression of Change (PGIC) questionnaire.

Exploratory health economic assessments during the study may include but not necessarily be limited to: the number of COVID-19-related inpatient hospital admissions; number of COVID-19-related physician and rehabilitation visits (in person office/telephonic and/or virtual visits); discharge location (home, skilled nursing facility); Number of ICU admissions; subject use of palliative and supportive care; and documentation of healthcare indirect costs through the Work Productivity and Activity

Duration of Treatment

Treatment duration is up to 28 days except where subjects are discontinued from the study if they withdraw consent, cannot comply with the schedule of treatment and evaluations in the study or if the investigator judges that further therapy is no longer in the subject's best interest. Patients may continue on treatment if, in the judgment of the treating physician, there would be clinical benefit.

### Example 2: COVID-19 Prophylaxis

The present Example describes preventative treatment of subjects that have a high risk of being infected with an embodiment of a therapy as provided herein. In particular, the present Example utilizes a six component embodiment of a combination therapy as previously described in International Publication WO 2014/169221A2 (the contents of which are hereby incorporated by reference). In the present Example, NED-260 refers to a combination of therapeutically effective amounts of agents comprising alpha-lipoic acid, curcumin, genistein, melatonin, metformin, and naltrexone. In the present Example, patients receiving treatment will be administered a combination of agents at the following doses:
R-Alpha-Lipoic Acid Sodium Salt: Dose 600 mg twice daily; after breakfast and dinner.
Curcumin: 500 mg three times daily; after meals.
Genistein: 250 mg twice daily; after breakfast and dinner.
Melatonin: 10 mg daily; at bedtime.
Metformin: 500 mg twice daily orally; after breakfast and dinner.
Naltrexone: 3.5 mg/day orally; at bedtime.

Study Objectives, Population, and Number of Patients

The present example comprises a multi-center, randomized, placebo controlled study of safety and efficacy of the provided embodiment of the present disclosure in the prophylactic treatment of COVID-19 negative and COVID-19 positive first responders as measured by IgG and IgM conversion.

In the absence of specific and near-term availability of antiviral treatment to stem the spread of COVID-19, attention should be given to prevention. Personal protection equipment supplies have been shown to be inadequate in the ongoing pandemic. In addition, PPE may be insufficiently protective. First responders and healthcare workers are significantly at risk of infection resulting from the immediate and frequent contact with the COVID-19 infected community and with infected colleagues and patients.

Reportedly, it will take a year or more before vaccines against SARS-CoV-2 become available, making prophylaxis a near term option in this setting. NED-260, an oral drug, is comprised of agents documented to affect viral, inflammatory and immune pathways specific to coronavirus which may be taken safety by the patient at home.

The primary objectives of this study include (1) reduction in the rate of seroconversion after 1 and 2 months dosing as measured by antibody response, and (2) reduction in the length of disease &/or severity of disease for subjects taking NED-260 recently testing IgM positive/IgG negative for COVID-19. The secondary objectives of this study include (1) safety and tolerability, and (2) reduction in length of disease &/or severity of disease in patients who convert to COVID-19 positive status over the test period of 2 months. The cohorts of this study comprise adult subjects working as first responders (such as police officers, paramedics, firefighters, and members of the military) and healthcare workers (such as physicians, nurses, and physician assistants) who have a high risk of contracting COVID-19 because of daily work-related exposure to COVID-19 positive patients.
Cohort A: (n=150) IgM Negative, IgG Negative who have comorbid conditions that place them into a high-risk category. First responders with high risk of contracting COVID-19 testing negative for COVID-19. Patients will be stratified by high and low risk by pre-defined criteria.
Cohort B: (n=150) Cohort A control
Cohort C: (n=150) IgM negative, IgG negative who do not have high-risk comorbid conditions
Cohort D. (n=150) Cohort C control
Cohort E. (n=150) IgM positive, IgG negative
Cohort F. (n=150) Cohort E control
Cohort G. (n=150) IgM positive, IgG negative who do not have high-risk comorbid conditions
Cohort H. (n=150) Cohort G control

Inclusion Criteria
1. Signed informed consent.
2. Subjects >18 years of age.
3. Negative IgM and negative IgG for cohort A, B, C, and D
4. Positive IgM, negative IgG for cohort E, F, G, and H
5. Able to comply with 4 separate daily oral dosing times (once each at breakfast, lunch, dinner, and before bed) including 16 pills daily and not have gastrointestinal disorders that would prevent/compromise the ingestion of NED-260 study drugs.
6. Patients must be able to orally swallow medications
7. Acceptable organ function as defined below:

A. Absolute neutrophil count >1,500 cells/μl.
B. Platelets >100,000 cells/μl.
C. Hemoglobin ≥9.0 g/dL
D. AST/ALT≤2.5 times the upper limit of normal.
E. Serum creatinine <1.5×the upper limit of normal (ULN) or if higher than the institutional normal range, calculated creatinine clearance (CrCl) must be ≥50 mL/min/1.73 m2 (e.g., by Cockcroft-Gault formula); actual body weight must be used for CrCl unless body mass index (BMI) is >30 kg/m2, in which case, lean body weight must be used.
8. Women of child-bearing potential (defined as a woman who has not undergone surgical sterilization or who has not been naturally post-menopausal for at least 24 consecutive months if ≤55 years, or at least 12 consecutive months if >55 years) must have a negative serum pregnancy test prior to the first dose of study drug. Male and female subjects with reproductive potential must agree to use an effective method of contraception throughout the study and for 90 days after discontinuation of NED-260.

Exclusion Criteria
1. Major surgery (excluding minor procedures, e.g., placement of vascular access, gastrointestinal/biliary stent, biopsy)<4 weeks prior to planned first day.
2. Subjects with acute or chronic metabolic acidosis.
3. Subject must be able to temporarily discontinue metformin at the time of or prior to any radiologic study involving the use of intravascular iodinated contrast materials. Metformin should be withheld 48 hours subsequent to the procedure and reinstituted only after renal function has been re-evaluated and found to be normal.
4. Participation in any other clinical investigation using an experimental drug within 4 weeks of the first dose of any component of NED-260.
5. Uncontrolled intercurrent illness including, but not limited to ongoing or active symptomatic congestive heart failure, unstable angina pectoris, cardiac arrhythmia, or psychiatric illness/social situations that would limit compliance with study requirements, as determined by the investigator.
6. NOTE that patients with diabetes will be allowed on study but will require a physician-Sponsor consult regarding reconciled and optimal metformin dosing (see metformin prescribing information).
7. Women who are pregnant or breastfeeding.
8. Known contraindication or drug-interaction between active co-medication and any NED-260 component that in opinion of investigator will interfere with co-medication or NED-260 absorption, metabolism, or clinical activity.
9. Known allergy or intolerance to any NED-260 component.
10. Sponsor reserves right to exclude any subject from the study on basis of pre-study medical histories, physical examination findings, clinical laboratory results, prior medications, or other entrance criteria.

Study Design

All subjects will be followed for safety and toxicity throughout the study and for ≥28 days following NED-260 discontinuation.

All COVID-19-positive patients will require initial labs and review of medical history.
Cohort A: (n=150) participants who are IgM and IgG Negative for COVID-19 and who have comorbid conditions that place them into a high-risk category. First responders with high risk of contracting COVID-19 testing negative for COVID-19.
Cohort B: (n=150) Cohort A control
Cohort C: (n=150) participants who are IgM and IgG negative for COVID-19 and who do not have high-risk comorbid conditions
Cohort D. (n=150) Cohort C control
Cohort E. (n=150) participants who are IgM positive and IgG negative for COVID-19 and who have comorbid conditions that place them into a high-risk category
Cohort F. (n=150) Cohort E control
Cohort G. (n=150) participants who are IgM positive and IgG negative for COVID-19 and who do not have high risk comorbid conditions
Cohort H. (n=150) Cohort G control
Cohort A, C, E, and G participants who advance to hospitalization or emergency room evaluation would then be eligible for potential entry into Study NED-260-01-20.

Statistical Analysis

Clinical endpoints include vital signs including oximetry, respiratory rate, heart rate, and temperature curve.

Additional endpoints include total number of missed work days, total number of days to full recovery, number of hospital admissions, number of ICU admissions, and subjective self-assessment of severity of disease, and death.

Ratings for the degree of severity for common symptoms from a 1 to 4 or 1 to 10 level on a daily basis or even twice a day basis.

Safety endpoints employed will include but not necessarily be limited to number and % of subjects experiencing: adverse events of any grade; grade 3-5 adverse events; adverse events of any grade considered by investigator to be at least possibly related to NED-260; grade 3-5 adverse events considered by investigator to be at least possibly related to NED-260; serious adverse events requiring study discontinuation; NED-260 discontinuations or suspensions; SAEs at least possibly related to NED-260.

Quality of Life (QOL) assessments may include but not necessarily be limited to the Patient Global Impression of Change (PGIC) questionnaire.

Exploratory health economic assessments during the study may include but not necessarily be limited to: the number of COVID-19-related inpatient hospital admissions; number of COVID-19-related physician and rehabilitation visits (in person office/telephonic and/or virtual visits); discharge location (home, skilled nursing facility); Number of ICU admissions; subject use of palliative and supportive care; and documentation of healthcare indirect costs through the Work Productivity and Activity Impairment Questionnaire General Health V2.0 (WPAI: GH).

Duration of Treatment

Duration of treatment is 2 months; however, patients may continue on treatment if, in the judgment of the treating physician, there would be clinical benefit. Subjects are discontinued from the study if they withdraw consent, cannot comply with the schedule of treatment and evaluations in the study or if the investigator judges that further therapy is no longer in the subject's best interest.

### Example 3: Luciferase-Based SARS-CoV-2 Spike Protein Pseudovirus Neutralization Assay

The present Example describes testing of a NED-260 therapy embodiment, as described herein, to assess cytotoxicity and/or pseudovirus neutralization activity. In the present Example, the referenced NED-260 embodiment comprises a combination of therapeutically effective amounts of alpha-lipoic acid, curcumin, genistein, melatonin, metformin, and naltrexone. It will be understood by those skilled in the art, that embodiments tested in the present Example can be combined with other treatments, such as those described herein.

Materials and Methods

Vero cells were seeded in black 96-well plates on Day-1 at 5.00E+04 cells per well. Eight dilutions of a NED-260 embodiment were prepared in triplicate and incubated for 1-hour with approximately 5,000 RLU of rVSV-SARS-CoV-2; pseudovirus only and cells only were added for controls and calculation, and an internal assay control was also included for assay validation. A NED-260/virus mixture was then added to the Vero cells and the plates were incubated for 24-hours at 37° C. Firefly Luciferase activity was detected using the Bright-Glo™ Assay System kit (Promega). Fifty percent inhibition concentration (IC50) or dose/dilution (ID50) was calculated using XLfit dose response model. Dilutions of remdesivir was used as a negative control and dilutions of a SARS-CoV-2 Spike antibody was used as a positive control. All graphs show mean values and standard error of the mean.

As discussed above, eight dilutions of a NED-260 embodiment were tested, and all dilutions were made from a Test Agent 1 (“TA #1”) solution, also referred to as Dilution Factor 1 solution (see, e.g., Table 1 and Table 2). The eight dilution factors tested in the pseudovirus neutralization assay were as follows: 1:1 (Dilution Factor 1), 1:10, 1:20, 1:33.3, 1:100, 1:200, 1:1000, and 1:10,000 (see Table 2). Concentrations of individual compounds in dilution factor 1 were chosen based on estimated peak serum concentrations of NED-260 compounds.

TABLE 1
Preparation of a NED-260 embodiment for pseudovirus neutralization assays.

| | | | | | | | | Solvent final |
|---|---|---|---|---|---|---|---|---|
| TA | Compound | Source | Cat# | Lot#/Batch# | Solvent | MW | Mass (mg) | vol. (ml) |
| TA#1 | R-Alpha lipoic acid | Sigma | 07039-50MG | BCCC5807 | water | 206.33 | 15.9906 | 0.5 |
| | Curcumin | Indofine | 1403447 | 1403447 | ethanol | 368.39 | 1.6578 | 0.5 |
| | Melatonin | Indofine | 025638 | 20101101 | ethanol | 232.28 | 0.0087 | 0.5 |
| | Metformin | Selleck Chemical | S1950 | 10 | ethanol | 165.62 | 2.0703 | 0.5 |
| | Naltrexone | Sigma | N3136-1G | BCBX4989 | water | 377.86 | 0.0030 | 0.5 |
| | Genistein | LC Labs | CH-148 | CH-148 | DMSO | 270.23 | 0.0754 | 0.5 |

TABLE 2
Eight Dilutions of a NED-260 embodiment for use in pseudovirus neutralization assays.
Values in leftmost column indicate dilutions a NED-260 embodiment that was made
from original stock solution (“TA#1”, also referred to as Dilution Factor
1). All other columns are μM concentrations of each compound in NED-260 dilution
mixes. Every dilution was prepared fresh in 1% serum medium and immediately used.

| NED-260 | | | | | | |
|---|---|---|---|---|---|---|
| Treatment | R-Alpha | | | | | |
| Matrix | Lipoic Acid | Curcumin | Melatonin | Metformin | Naltrexone | Genistein |
| Dilution Factor: | | | | | | |
| 1:10000 | 0.31 | 0.018 | 0.00015 | 0.05 | 0.000032 | 0.00117 |
| 1:1000 | 3.1 | 0.18 | 0.0015 | 0.5 | 0.00032 | 0.0117 |
| 1:200 | 15.5 | 0.9 | 0.0075 | 2.5 | 0.0016 | 0.0585 |
| 1:100 | 31 | 1.8 | 0.015 | 5 | 0.0032 | 0.117 |
| 1:33.3 | 93 | 5.4 | 0.045 | 15 | 0.0096 | 0.351 |
| 1:20 | 155 | 9 | 0.075 | 25 | 0.016 | 0.585 |
| 1:10 | 310 | 18 | 0.15 | 50 | 0.032 | 1.17 |
| 1 | 3100 | 180 | 1.5 | 500 | 0.32 | 11.7 |

Results

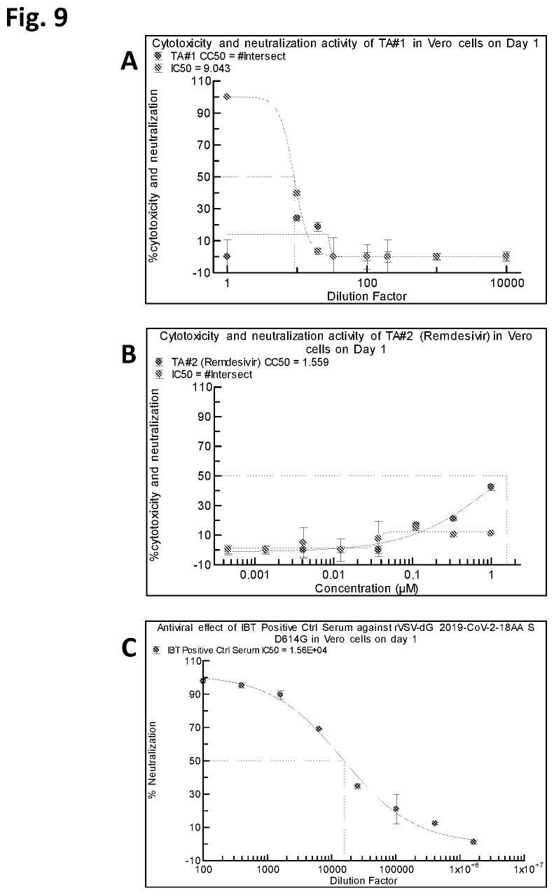

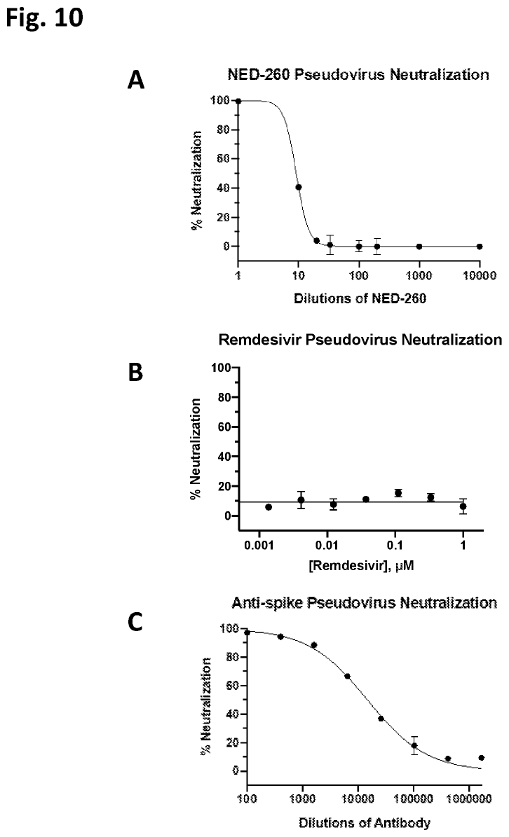

As measured by this assay, a tested embodiment of NED-260 reduced the amount of virus that was able to enter into cells by 50% with an IC50 of approximately 9.043 uM (FIG. 9A and FIG. 10A), a finding comparable to the effect of a monoclonal antibody against SARS-CoV-2 (FIG. 9C and FIG. 10C). Remdesivir did not show any effect on cellular entry (FIG. 9B and FIG. 10B). Notably, the drug dose of NED-260 that was able to achieve this 50% reduction in cellular entry is within an achievable concentration for patient dosing. Furthermore, tested embodiments of NED-260 exhibited low cellular toxicity at all concentrations tested supporting the known safety profile (FIG. 9A). These findings support use of NED-260 a safe, potent anti-SARS-CoV-2 agent, with efficacy over that of other therapeutic options (e.g., remdesivir).

### Example 4: SARS-CoV-2 Plaque Reduction Assay

The present Example describes testing of a NED-260 therapy embodiment, as provided herein, in a SARS-CoV-2 plaque reduction assay. In the present Example, the referenced NED-260 embodiment comprises alpha-lipoic acid, curcumin, genistein, melatonin, metformin, and naltrexone.

Materials and Methods

Confluent Vero E6 cells were seeded in 6-well plates. Two-fold serial dilutions of a NED-260 embodiment were pre-incubated with about 30 plaque forming units (PFU) of SARS-CoV-2/Quebec City/21697/2020 for 60 minutes at 37° C. in a 5% CO2 incubator. The pre-incubated NED-260 embodiment and virus mix was added to confluent Vero E6 cells (in triplicate) and incubated for 60 minutes at 37° C. in a 5% CO2 incubator. Infected cells were incubated for 4-days in 2× Minimal Essential Medium supplemented with 2% fetal bovine serum containing 0.6% SeaPlaque agarose. After 4-days of incubation, cells were fixed with formaldehyde 4% and stained with crystal violet. The number of PFUs were counted under an inverted microscope and plotted against the logarithm of antiviral concentrations. All graphs show mean values and standard error of the mean.

Seven dilutions of a NED-260 embodiment were tested in the plaque reduction assay, and all dilutions were made from a NED-260 Dilution Factor 1 solution (see, e.g., Table 3). NED-260 dilution factors tested in the plaque reduction assay were as follows: 1:1 (dilution factor 1), 1:2, 1:32, 1:64, 1:128, 1:256, and 1:1024. Concentrations of individual compounds in dilution factor 1 were chosen based on estimated peak serum concentrations of NED-260 compounds.

TABLE 3
Preparation of a NED-260 embodiment for plaque reduction assay

| | | R-Alpha | | | | | | | | | | | | 2% FBS in |
|---|---|---|---|---|---|---|---|---|---|---|---|---|---|---|
| | | Lipoic Acid | | Curcumin | | Melatonin | | Metformin | | Naltrexone | | Genistein | | MEM |
| Mr | (g/moL) | 206.33 | | 368.39 | | 232.28 | | 165.62 | | 377.86 | | 270.24 | | na |
| Concentration | (μM) | 3100 | | 180 | | 1.5 | | 500 | | 0.32 | | 11.7 | | na |
| | (μg/mL) | 639.62 | | 66.31 | | 0.35 | | 82.81 | | 0.12 | | 3.16 | | na |
| Dilution | Fold | 10 | | 10 | | 1,000 | | 10 | | 10,000 | | 100 | | na |
| Sotck Conc.: | (μg/mL) | 6396.2 | | 663.1 | | 348.4 | | 828.1 | | 1209.2 | | 316.2 | | na |
| | Weigh in: | 64 | mg | 6.63 | mg | 6.97 | mg | 8.28 | mg | 12.09 | mg | 6.32 | mg | na |
| | Volume: | 0.064 | g | 0.00683 | g | 0.00697 | g | 0.00828 | g | 0.01209 | g | 0.00632 | g | |
| | | 10 | mL | 10 | mL | 20 | mL | 10 | mL | 10 | mL | 20 | mL | na |
| Intermittent | Stock: | | | | | 1 | mL | | | 1 | mL | | | |
| Dilution 1 | Dilutent | | | | | 9 | mL | | | 9 | mL | | | |
| Intermittent | Stock: | | | | | | | | | 1 | mL | | | |
| Dilution 2 | Dilutent | | | | | | | | | 9 | mL | | | |

| | | | | | | | | | | | | | |
|---|---|---|---|---|---|---|---|---|---|---|---|---|---|
| Final Volume | 1.0 | mL | 1.0 | mL | 100 | μL | 1.0 | mL | 100 | μL | 100 | μL | 6.7 mL |

Results

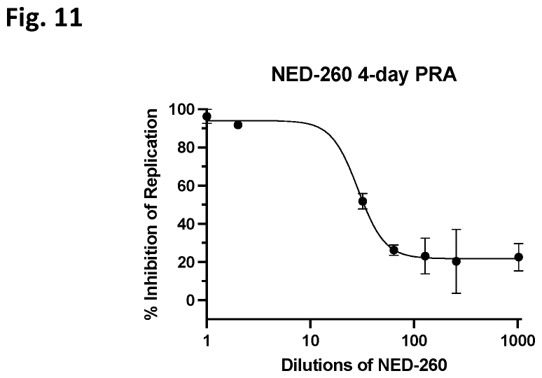

The present example demonstrates that embodiments of NED-260 was able to inhibit replication/cellular entry of SARS-CoV-2 from about 70% to about 100% at the end of four days compared to a vehicle control (FIG. 11). Further, cytotoxicity was minimal at all NED-260 concentrations tested. Combined, the results of these experiments demonstrate the utility of NED-260 therapies, as described herein, for use in treatment of viral infection, in particular, SARS-CoV-2 infection.

### EQUIVALENTS

Those skilled in the art will recognize, or be able to ascertain using no more than routine experimentation, many equivalents to the specific embodiments described herein. The scope of the present disclosure is not intended to be limited to the above Description, but rather is as set forth in the following claims.

1. (canceled)

2. A method of treating or preventing a disease or condition associated with a viral infection in a subject, the method comprising a step of administering to the subject a combination of agents, wherein the combination of agents comprises three or more of alpha lipoic acid, curcumin, genistein, melatonin, metformin, and naltrexone.

3. (canceled)

4. The method of claim 1, wherein the viral infection is caused by a virus of a viral family selected from Reovirus, Picornavirus, Coronavirus, Flavivirus, Togavirus, Filovirus, Orthomyxovirus, Rhabdovirus, and Retrovirus.

5. The method of claim 1, wherein the virus is a negative-sense single stranded RNA virus.

6. The method of claim 1, wherein the virus is a coronavirus.

7. The method of claim 1, wherein the virus is SARS-CoV-2.

8. The method of claim 1, wherein the subject is infected with virus prior to receiving treatment.

9. The method of claim 1, wherein the subject receives treatment prior to exposure to virus.

10. The method of claim 1, wherein the agents are administered to the subject according to a personalized weekly or monthly regimen.

11.-14. (canceled)

15. The method of claim 1, wherein the combination further comprises one or more additional anti-viral therapies.

16. The method of claim 1, wherein the combination further comprises one or more of Abacavir sulfate, Acyclovir, Bevacizumab, Bromelain, Bromhexine hydrochloride, Chloroquine phosphate, Danoprevir, Delaviridine mesylate, Didanosine, Dipyridamole, Ebastine, Entecavir, Favipiravir, Ganciclovir sodium, Hydroxychloroquine sulfate, Interferon alpha, Intravenous immunoglobulin, Lamivudine, Lopinavir, Ritonavir, Methylprednisolone, Nelfinavir mesylate, Nivolumab, Recombinant human ACE2, Remdesivir, Remestemcel-L, Saquinavir mesylate, Sildenafile citrate, Stavudine, Tenofovir disoproxil fumarate, Thymosin, Thalidomide, Umifenovir, Zanamivir, Zidovudine, Niclosamide, Famotidine, and combination thereof.

17. The method of claim 1, wherein the combination comprises alpha lipoic acid at a dose of about 600-1200 mg/day.

18. (canceled)

19. The method of claim 1, wherein the combination comprises curcumin at a dose of about 1000-3000 mg/day.

20. (canceled)

21. The method of claim 1, wherein the combination comprises genistein at a dose of about 60-600 mg/day.

22. (canceled)

23. The method of claim 1, wherein the combination comprises metformin at a dose of about 250-2000 mg/day.

24. (canceled)

25. The method of claim 1, wherein the combination comprises melatonin at a dose of about 1-40 mg/day.

26. (canceled)

27. The method of claim 1, wherein the combination comprises naltrexone at a dose of about 3-5 mg/day.

28. (canceled)

29. The method of claim 1, wherein one or more of the agents is formulated for oral administration.

30. The method of claim 1, wherein one or more agents are formulated for buccal administration, sub-labial administration, sub-lingual administration, or a combination thereof.

31.-32. (canceled)

33. The method of claim 1, wherein two or more agents are formulated together as an admixture.

34.-38. (canceled)

39. A combination comprising:
(i) three or more agents comprising alpha lipoic acid, curcumin, genistein, melatonin, metformin, and naltrexone; and
(ii) one or more additional anti-viral therapies.

40.-74. (canceled)

75. The method of claim 1, wherein the agents are formulated as separate co-agents.
